SEQUENCE LISTING (57 sequences)

SEQ ID NO: 1 - DNA, 30 bp, Caenorhabditis elegans
ggatgcatgcaacaacaatcaaaaccatag

SEQ ID NO: 2 - DNA, 44 bp, Caenorhabditis elegans
ggccaatcccggggatcctctagacaagaatcagagaaaggaca

SEQ ID NO: 3 - DNA, 39 bp, Caenorhabditis elegans
aattaaccctcactaaaggtcaaagactcaccgcatacg

SEQ ID NO: 4 - DNA, 20 bp, Caenorhabditis elegans
gtactgatcgttgccggatt

SEQ ID NO: 5 - DNA, 20 bp, Caenorhabditis elegans
gcaatgtcagcacgtgattt

SEQ ID NO: 6 - DNA, 20 bp, Caenorhabditis elegans
cgaggatttttcccacaaaa

SEQ ID NO: 7 - DNA, 20 bp, Caenorhabditis elegans
ttcacacgatgaagctcgac

SEQ ID NO: 8 - DNA, 20 bp, Caenorhabditis elegans
taatacgactcactataggg

SEQ ID NO: 9 - DNA, 18 bp, Caenorhabditis elegans
ctgtgcttattggaacta

SEQ ID NO: 10 - DNA, 18 bp, Caenorhabditis elegans
acaaagtgcatcgtagta

SEQ ID NO: 11 - DNA, 18 bp, Caenorhabditis elegans
tgggcaataactacacaa

SEQ ID NO: 12 - DNA, 18 bp, Caenorhabditis elegans
atattttcaaggtcatcg

SEQ ID NO: 13 - DNA, 18 bp, Caenorhabditis elegans
gaggcccaatccaagaga

SEQ ID NO: 14 - DNA, 20 bp, Caenorhabditis elegans
tcttgcgttgtcatgctggt

SEQ ID NO: 15 - DNA, 20 bp, Caenorhabditis elegans
ttctgtgcttttgggatacc

SEQ ID NO: 16 - DNA, 20 bp, Caenorhabditis elegans
cgctgcgtgtttaagttggc

SEQ ID NO: 17 - DNA, 20 bp, Caenorhabditis elegans
tcggcaattttgagttggtt

SEQ ID NO: 18 - DNA, 19 bp, Caenorhabditis elegans
ttccctaatttgacatggt

SEQ ID NO: 19 - DNA, 20 bp, Caenorhabditis elegans
ctgaccgtcggaagtgttat

SEQ ID NO: 20 - DNA, 20 bp, Caenorhabditis elegans
ggactcggcttcagacttat

SEQ ID NO: 21 - DNA, 20 bp, Caenorhabditis elegans
tatgacagtgccagtttggg

SEQ ID NO: 22 - DNA, 20 bp, Caenorhabditis elegans
tccgaaaagtgatgtttatg

SEQ ID NO: 23 - DNA, 20 bp, Caenorhabditis elegans
ctctggcgaccttgttgaag

SEQ ID NO: 24 - DNA, 18 bp, Caenorhabditis elegans
tactccgccgatctcgtg

SEQ ID NO: 25 - DNA, 18 bp, Caenorhabditis elegans
tgttggaaggtggagagg

SEQ ID NO: 26 - DNA, 21 bp, Caenorhabditis elegans
gcatgagcgttgaatctctcc

SEQ ID NO: 27 - DNA, 23 bp, Caenorhabditis elegans
aatgagatttcagctctgtcttt

SEQ ID NO: 28 - DNA, 20 bp, Caenorhabditis elegans
atgtcgaaggcttcgcatct

SEQ ID NO: 29 - DNA, 20 bp, Caenorhabditis elegans
gagccatgatgaccttctct

SEQ ID NO: 30 - protein, 20 aa, Caenorhabditis elegans
GCIAACGGITAYAAYTTYGG

SEQ ID NO: 31 - DNA, 20 bp, Caenorhabditis elegans
  unsure (12): N = A,G, C or T. Sequence represents a degenerate oligonucleotide so unknown which nucleotide is at position 12.
acttgcgaatcntgyaargc

SEQ ID NO: 32 - protein, 21 aa, Caenorhabditis elegans
CATICCIACIGCRAARCAYTT

SEQ ID NO: 33 - DNA, 31 bp, Caenorhabditis elegans
  unsure (30)..(31): N = A, G, C or T.  N indicates exact nucleotide varies depending on future use of primer.
ggggacaagtttgtacaaaaaagcaggctnn

SEQ ID NO: 34 - DNA, 30 bp, Caenorhabditis elegans
  unsure (30): N = A, G, C, or T. N indicates exact nucleotide varies depending on future use of primer.
ggggaccactttgtacaagaaagctgggtn

SEQ ID NO: 35 - DNA, 49 bp, Caenorhabditis elegans
ggggacaagtttgtacaaaaaagcaggcttacatcgtatgcagagaaac

SEQ ID NO: 36 - DNA, 47 bp, Caenorhabditis elegans
ggggaccactttgtacaagaaagctgggtttatgatgccatgtacgg

SEQ ID NO: 37 - protein, 201 aa, Chicken Gallus
LPMLPHLADLVSYSIQKVLGFAKMIPGFRDLTAEDGLALLKSSAIEVIMLRSNQSFTMEDMSWTCGSNDFKYKVSDVTQAGHSMDLLEPLVKFQVGLKKLNLHEEEHVLLMAICILSPDRPGVQDTSLVESIQDRLSDIIQTYLRCRHPPPGSRLLYAKMIQKIADLRSLNEEHSKGYRCLSFQPEHSMQLTPLVLEVFGN

SEQ ID NO: 38 - protein, 201 aa, Human
LSMLPHLADLVSYSIQKVLGFAKMIPGFRDLTSEDQLVLLKSSAIEVIMLRSNESFTMDDMSWTCGNQDYKYRVSDVTKAGHSLELIEPLLKFQVGLKKLNLHEEEHVLLMAICIVSPDRPGYQDAALIEAIQDRLSNTLQTYLRCRHPPPGSHLLYAKMIQKLADLRSLNEEHSKGYRCLSFQPECSMKLTPLVLEVFGN

SEQ ID NO: 39 - protein, 201 aa, Xenopus laevis
LSMIPHIADLVSYSIQKILGFAKMIPGFRDLLAEDQIALLKSSVIEVIMLRSNQSFSLDDMSWTCGSEDFKYKVDDVTQAGHNMELLEPLVKFQVGLKKLDLHEEEHVLLMAICILSPDRPGLQDKALVESIQDRLSSTLQTYLLCKHPPPGSRLLYAKMIQKLADLRSLNEEHSKGYRSISFLPEHSMKLTPLMLEVFSD

SEQ ID NO: 40 - protein, 192 aa, musculus
  UNSURE (124): Xaa = any amino acid naturally occurring in mice. i.e. any of the standard 20.
IPLLPHLADVSTYMRKGVLNFAKVISYFRDLPIEDQLSLLKGATFEMCILRFNTMFDTETGTWECGRLAYCFEDPNGGFQKLLLDPLMKFHCMLKKLQLHKEEYVLMQAISLFSPDRPGVVQRXVVDQLQERFALTLKAYLECSRPYPAHRFLKIMAVITELRSINAQQTQQLLRIQDSHPFATPMQELFSS

SEQ ID NO: 41 - protein, 197 aa, Xenupus Laevis
FLMLPHISDLVTYMIKGIISFAKMLPYFKSLDLEDGLALLKGSVAEVSVIRFNTVFNSDTNTWECGPFTYDTEDMFLAGFRQLFLEPLVRIHRMMRKLNLQSEEYAMMAALSIFASDRPGYCDWEKIQKLGEHIALTLKDFLDSQRPPSPQNRLLYPKIMECLTELRTVNDIHSKQLLEIWDIQPDATPLMREVFGS

SEQ ID NO: 42 - protein, 194 aa, Human
LPLVTHFADINTFMVLQVLKFTDKLPVFRSLPIEDGLSLLKGAAVEICHIVLNTTFCLQTQNFLCGPLRYTIEDGARVGFQVEFLELLFHFHGTLRKLQLQEPEYVLLAAMALFSPDRPGVTQRDEIDQLGEEMALTLQSYLKGQQRRPRDRFLYAKLLGLLAELRSINEAYGYGIQHIQGLSAMMPLLQEICS

SEQ ID NO: 43 - protein, 199 aa, Caenorhabditis elegans
PKSIDGVFRIIEEALRRIVNMACQLSLFRELHVDDRKNLLKSGFGELLIVRGLMAYDKSDNSWNHSFGVRGKMEVKVEVLKNPKLEEHYKAHMNLLSTFGEDVRNNEHLMLIFNAAVIFHPHVSNLRDSKRVHSTQAKYFQMLLKLLTFEYGKSRADIAYSNLLNQVVELHRVNRTLLRVFYGLDIAQLDPIIRELCSF

SEQ ID NO: 44 - protein, 198 aa, drosophile
NPKLLQLINLTAVAIKRLLKMAKKITAFRDMCQEDGVALLKGGCTEMMIMRSVMIYDDDRAAWKVPHTKENMGNIRTDLLKFAEGNIYEEHQKFITTFDEKWRMDENIILIMCAIVLFTSARSRVIHKDVIRLEQNSYYYLLRRYLESVYSGCEARNAFIKLIQKISDVERLNKFIINVYLNVNPSQVEPLLREIFDL

SEQ ID NO: 45 - protein, 205 aa, melanogaster
MEQGPDIMNIMDVTMRRFVKVAKGVPAFREVSQEGKFSLLKGGMIEMLTVRGVTRYDASTNSFKTPTIKGQNVSVNVDDMFAKLNANAQAQKAKCLEFFGFFDEEIKKNELAVYLVMLAVLFSVRDSPPMNENDVRIVTERHNHFMSLLNRYLESLFGEAQRRIFERIPKALGLLNEIARNAGMLFMGTVRSGEAEELPGEFFKI

SEQ ID NO: 46 - DNA, 23 bp, Caenorhabditis elegans
gtggttagttagagtttaaaata

SEQ ID NO: 47 - DNA, 19 bp, Caenorhabditis elegans
ggtaatcacgtgctgacat

SEQ ID NO: 48 - protein, 94 aa, Bombyx mori
ELCPVDGDKVSGYHYGLLTCESCKGFFKRTVQNKKVYTCVAERACHIDKTQRKRCPFCRFQKCLDVGMKLEAVRADRMRGGRNKFGPMYKRDRA

SEQ ID NO: 49 - protein, 94 aa, Drosophila melanogaster
ELCPVCGDKVSGYHYGLLTCESCKGFFKRTVQNKKVYTCVAERSCHIDKTQRKRCPYCRFQKCLEVGMKLEAVRADRMRGGRNKFGPMYKRDRA

SEQ ID NO: 50 - protein, 94 aa, Metapenaeus ensis
ELCPVCGDKVSGYHYGLLTCESCKGFFKRTVQNKKVYTCVADRSCQIDKTQRKRCPYCRFQKCLEVGMKLEAVRADRMRGGRNKFGPMYKRDRA

SEQ ID NO: 51 - protein, 94 aa, Gallus gallus
ELCPVCGDKVSGYHYGLLTCESCKGFFKRTVQNNKRYTCIENQNCQIDKTQRKRCPYCRFQKCLSVGMKLEAVRADRMRGGRNKFGPMYKRDRA

SEQ ID NO: 52 - protein, 93 aa, Humans
ELCPVCGDKVSGYHYGLLTCESCGFFKRTVQNNKRYTCIENQNCQIDKTQRKRCPYCRFQKCLSVGMKLEAVRADRMRGGRNKFGPMYKRDRA

SEQ ID NO: 53 - protein, 94 aa, Xenopus laevis
ELCPVCGDKVSGYHYGLLTCESCKGFFKRTVQNNKRYTCIENQSCPIDKTQRKRCPYCRFQKCLSVGMKLEAVRADRMRGGRNKFGPMYKRDRA

SEQ ID NO: 54 - protein, 94 aa, Brachydanio rerio
EMCPVCGDKVSGYHYGLLTCESCKGFFKRTVQNNKRYTCIENQSCQIDKTQRKRCPYCRFQKCLTVGMKLEAVRADRMRGGRNKFGPMYKRDRA

SEQ ID NO: 55 - protein, 94 aa, Gallus gallus
ELCPVCGDKVSGYHYGLLTCESCKGFFKRTVQNNKHYTCTESQNCKIDKTQRKCCPYCRFQKCLTVGMRLEAVRADRMRGGRNKFGPMYKRDRA

SEQ ID NO: 56 - protein, 94 aa, Humans
ELCPVCGDKVSGYHYGLLTCESCKGFFKRTVQNNKHYTCTESQSCKIDKTQRKRCPFCRFQKCLTVGMRLEAVRADRMRGGRNKFGPMYKRDRA

SEQ ID NO: 57 - protein, 94 aa, Caenorhabditis elegans
EMCPVCGDRVSGYHYGLLTCESCKGFFKRTVQNKKQYQCSAEANCHVDRTCRKRCPSCRFQKCLTMGMKMEAVRADRMRGGRNKFGSFYKKDRA

(19) United States
(12) Patent Application Publication
(10) Pub. No.: US 2004/0213771 A1
(43) Pub. Date: Oct. 28, 2004

(54) Targets and screens for agents useful in controlling parasitic nematodes

(76) Inventors: Ann E. Sluder, Burlington, MA (US); Christopher Provost, Woburn, MA (US); Tim Lindblom, Batesville, AR (US); Marie Sutherlin Causey, Cambridge, MA (US); Leo X. Liu, Weston, MA (US)

Correspondence Address: Choate, Hall & Stewart, Exchange Place, 53 State Street, Boston, MA, 02109, US

(21) Appl. No.: 10/102,204

(22) Filed: Mar. 20, 2002

Related U.S. Application Data
(60) Provisional application No. 60/277,339, filed on Mar. 20, 2001.

(51) Int. Cl. A61K 39/002; A61K 38/43

(52) U.S. Cl. 424/94.1; 424/265.1

(57) ABSTRACT

The invention provides methods and reagents for identifying compounds that exhibit anti-nematode activity and compounds that potentiate the effects of other anti-nematode agents. The invention provides methods identifying modulators of nematode xenobiotic sensing nuclear receptors. The invention also provides methods for screening for anti-nematode compounds using sensitized nematode strains and further provides such sensitized strains. The invention further provides methods and screens for identifying new biological targets for anti-nematode compounds, e.g., for identifying nematode genes and proteins towards which anti-nematode compounds can be developed. The invention provides methods for controlling parasitic nematodes using compounds identified in the inventive screens. In addition, the invention provides sensitized nematode strains and methods for using such strains to enhance identification of compounds in the context of a wide variety of nematode model screening systems and strains.

### CROSS-REFERENCE TO RELATED APPLICATION

This application claims priority to U.S. provisional application U.S.S.No. 60/277,339 entitled “Targets and Screens for Agents Useful in Controlling Parasitic Nematodes”, filed Mar. 20, 2001, which is herein incorporated by reference.

### BACKGROUND OF THE INVENTION

Nematodes are elongated symmetrical roundworms that constitute one of the largest and most successful phyla in the animal kingdom. Many nematode species are free-living and feed on bacteria, whereas others have evolved into parasites of plants and animals, including humans. Human infections with parasitic nematodes are among the most prevalent infections worldwide. Over one billion people, predominantly in tropical and subtropical developing countries, are infected with soil and vector-borne nematodes that cause a variety of debilitating diseases (Liu L. X., Weller P. F., Intestinal Nematodes. Chapter 181, in Harrison's Principles of Internal Medicine, Isselbacher K J, Braunwald E, Wilson J D, Martin J B, Fauci A S, Kasper D L, eds., New York: McGraw-Hill, pp. 916-920, 1994). Among these parasitic nematodes are Ancylostoma and Necator hookworms that cause anemia and malnutrition, Ascaris roundworms that can cause pulmonary and nutritional disorders, and Strongyloides stercoralis which can cause potentially lethal Nematodes of the order Spirurida are responsible for onchocerciasis (river blindness) and lymphatic filariasis. Animal parasitic nematodes infect a wide variety of both domestic and wild animals. Major animal pathogens include Haemonchus contortus, which infects herbivorous vertebrates, Trichinella spiralis, the causative agent of trichinosis, and various members of the order Ascaridida, which infect pigs and dogs in addition to humans.

Plant parasitic nematodes also represent major problems, being responsible for many billions of dollars in economic losses annually. The most economically damaging plant parasitic nematode genera belong to the family Heterderidae of the order Tylenchida, and include the cyst nematodes (genera Heterodera and Globodera) and the root-knot nematodes (genus Meloidogyne). The soybean cyst nematode (H. glycines) and potato cyst nematodes (G. pallida and G. rostochiensis) are important examples. Root-knot nematodes infect thousands of different plant species including vegetables, fruits, and row crops. In contrast to many viral and bacterial pathogens, little is known about the molecular basis of nematode parasitism, limiting the available framework for rational anthelminthic (anti-nematode) drug development (David J. R., Liu L. X., Molecular biology and immunology of parasitic infections, Chapter 170 in Harrison's Principles of Internal Medicine, Isselbacher K J, Braunwald E, Wilson J D, Martin J B, Fauci A S, Kasper D L, eds., New York: McGraw-Hill, pp. 865-871, 1994).

Anti-nematode drug or pesticide discovery has traditionally relied either on direct screening of compounds against whole target organisms or on chemical modification of existing compounds, strategies that have resulted in relatively few classes of active agents acting against a limited number of known biological targets. For example, organophosphates and carbamates, the oldest extant class of nematicides, were developed many decades ago and target a single, biologically conserved enzyme, acetylcholinesterase. Imidazole derivatives such as benzimidazole exert their antiparasitic effects by binding tubulin. Levamisole acts as an agonist on the nicotinic acetylcholine receptor, and avermectins act as irreversible agonists at glutamate-gated chloride channels (Liu L. X., Weller P. F., Drug Therapy: Antiparasitic Drugs, N Engl J Med 334, 1178-1184, 1996). Unfortunately, there are certain debilitating nematode infections which are difficult if not impossible to cure with existing therapeutics. For example, in onchocerciasis, the adult female Onchocerca volvulus worms are refractory to even newer generation drugs (Liu L. X., Weller P. F., Drug Therapy: Antiparasitic Drugs, N Engl J Med 334, 1178-1184, 1996). In addition, drug resistance has emerged to all of these main classes of therapeutics, particularly in livestock animal applications in which their use is widespread (Sangster N. C., Gill J., Pharmacology of anthelmintic resistance, Parasitol Today 15, 141-146, 1999). Thus far it has not been possible to develop effective and practical vaccines, and even if such vaccines become available, effective anti-nematode drugs will still be needed for treating established infections as well as offering the potential advantages of prophylaxis and treatment for a broad spectrum of nematode parasites.

The drawbacks of existing agents that are currently used to control plant parasitic nematodes are equally or more significant. Fumigant nematicides such as methyl bromide and 1,3-dichloropropene, which kill nematodes by slowly diffusing through the soil, are phytotoxic and must be applied well before planting. Environmental concerns, primarily groundwater contamination, ozone depletion, and pesticide residues in food (National Research Council, Pesticides in the Diet of Infants and Children (Washington, D.C.: National Academy of Sciences, 1993) have prompted the removal of Aldicarb, DGBCP, and other toxic nematicides from the market by the Environmental Protection Agency, with methyl bromide to be withdrawn in the U.S. by 2002 (Johnson, S. L., Bailey, J. E., “Pesticide Risk Management and the United States Food Quality Protection Act of 1996“, in Pesticide Chemistry and Bioscience: The Food-Environment Challenge, Brooks, G. T. and Roberts, T. R., (eds.), Cambridge: Royal Society of Chemistry, pp. 411-420, 1999). Physical control measures (such as solarization and hot water treatment), biological control measures (e.g., crop rotation), and integrated approaches have been used to ameliorate the damage caused by plant parasitic nematodes (Whitehead, A. G., Plant Nematode Control, Wallingford: CAB International, 1998), but no single method or combination of measures is uniformly effective.

Because of the rapidly increasing limitations of existing products, there is a need for innovation in anthelminthic discovery. There exists an urgent need for new agents active against pathogenic and parasitic nematode species, e.g., compounds active against animal or plant parasitic nematodes. To facilitate the discovery of new anti-nematode compounds, there exists a need for the identification and validation of additional biological targets (e.g., nematode genes and proteins) against which such compounds can be directed. Furthermore, there exists a need for the development of new methodologies and screening technologies for the identification of compounds active against nematodes. In particular, there exists a need for the development of screening assays that can be conveniently performed in a high throughput format.

### SUMMARY OF THE INVENTION

The present invention addresses the foregoing needs by providing methods and reagents for identifying compounds that exhibit anti-nematode activity and compounds that potentiate the effects of other anti-nematode agents. The invention further provides methods for identifying new biological targets for anti-nematode compounds, e.g., for identifying nematode genes and proteins towards which anti-nematode compounds can be developed.

The practice of the present invention employs, unless otherwise indicated, conventional techniques of molecular biology, cell biology, recombinant DNA technology, microbiology, immunology, microscopy, and chemistry. Such techniques are explained in the literature. In addition, the present invention employs information on the biology, biochemistry, physiology, anatomy, etc., of C. elegans, and techniques known in the art for the propagation, study, manipulation, and storage of nematodes, particularly C. elegans, as described in Wood, W. B. and the Community of C. elegans Researchers, eds., The Nematode Caenorhabditis elegans, Cold Spring Harbor Laboratory Press, Cold Spring Harbor, N.Y., 1988; Riddle, D. L. et al., eds., C. elegans II, Cold Spring Harbor Laboratory Press, Cold Spring Harbor, N.Y., 1997; Epstein, H. and Shakes, D., eds., Caenorhabditis elegans: Modern Biological Analysis of an Organism, Methods in Cell Biology, Vol., 48, Academic Press, San Diego, 1995. The contents of all references cited in this document, including patents, patent applications, books, journal articles, etc., are herein incorporated by reference. Certain of these references are listed within the body of the document while others are listed at the end in the section entitled Reference List and are referred to by number herein. There are three reference lists, and references included on each list are indicated differently herein. References that appear in Reference List 1 are indicated with a superscript. References in Table 1-3 also refer to Reference List 1. References that appear in Reference List 2 are indicated with a number in parentheses. References that appear in Reference List 3 are indicated with a number in square brackets. In addition, the inventors' provisional patent application entitled SCREENS AND ASSAYS FOR AGENTS USEFUL IN CONTROLLING PARASITIC NEMATODES, filed Jan. 18, 2001, Serial No. 60/263,081, is incorporated herein by reference.

The invention provides methods and screens to identify modulators (e.g., antagonists or agonists) of nematode nuclear receptors (NRs), e.g., nematode xenobiotic sensing nuclear receptors and further provides compounds identified according to the inventive methods. One such method of identifying a modulator of a nematode nuclear receptor comprises steps of: (i) providing a cell, wherein the cell expresses a polypeptide comprising a ligand binding domain of a nematode nuclear receptor or a portion thereof and a DNA binding domain or a portion thereof, and wherein the cell contains a reporter comprising a reporter gene and a nucleic acid that comprises a binding site for the polypeptide, wherein the nucleic acid is operably linked to the reporter gene; (ii) contacting the cell with a test compound; and (iii) determining whether the amount or activity of a molecule encoded by the reporter is increased or decreased in the presence of the test compound, wherein an increase or decrease in the amount or activity of the molecule encoded by the reporter is an indication that the test compound is a modulator of a nematode nuclear receptor. Nematode nuclear receptors of interest include xenobiotic sensing NRs, for example, NRs in the NR1I/J subfamily of nuclear receptors. Such NRs include, for example, C. elegans NHR-8, NHR-48, and DAF-12 and parasitic nematode homologs, orthologs, or paralogs thereof. In certain embodiments of the invention the screen is performed in yeast.

The invention also provides methods and screens for identifying anti-nematode agents (e.g., nematicides) using sensitized nematode strains. One such method comprises steps of: (i) providing a sensitized nematode, wherein the sensitized nematode contains a mutation in a gene encoding a nuclear receptor; (ii) contacting the sensitized nematode with a test compound; and (iii) determining whether an indicator of nematode well-being is altered in the sensitized nematode in the presence of the test compound, wherein an alteration in the indicator of nematode well-being is an indication that the compound possesses anti-nematode activity. According to certain embodiments of the invention the indicator of nematode well-being is viability, growth, reproduction, or feeding of the sensitized nematode, and the alteration is a decrease in viability, growth, reproduction, or feeding of the sensitized nematode in the presence of the test compound. However, indicators of nematode well-being other than viability, growth, reproduction, or feeding may also be employed including, but not limited to, movement, defecation, or expression of a gene or protein associated with nematode well-being. The alteration may be a decrease or an increase, depending upon the particular indicator selected. Sensitized strains of interest include strains with a mutation in one or more xenobiotic sensing NRs, for example, NRs in the NR1I/J subfamily of NR. Such NRs include, for example, C. elegans NHR-8, NHR-48, and DAF-12 and parasitic nematode homologs, orthologs, or paralogs thereof. As discussed below, the inventors have shown that a strain with a mutation in the nhr-8 gene displays increased sensitivity to certain toxins.

The invention further provides methods and screens for identifying combinations of compounds with anti-nematode activity. One such method comprises steps of: (i) providing a nematode; (ii) contacting the nematode with a first compound that modulates activity of a xenobiotic sensing nuclear receptor; (iii) contacting the nematode with a second compound; and (iv) determining whether an indicator of nematode well-being, e.g., the viability, growth, reproduction, movement, or feeding of the nematode is altered in the presence of both the first compound and the second compound to a greater extent than in the presence of only the first compound or the second compound.

The invention provides compounds identified according to the methods described herein and methods of use for the compounds as pharmaceutical agents and/or anti-nematode agents. Thus the invention provides pharmaceutical compositions comprising a compound (e.g., a modulator of a nematode NR such as a xenobiotic sensing NR) identified according to a method for identifying a compound described herein and anti-nematode agents comprising a compound (e.g., a modulator of a nematode NR such as a xenobiotic sensing NR) identified according to one of the methods described herein. The invention provides various methods for using such compounds, e.g., by administering them to an individual in need thereof, by treating a plant or seed, etc.

The invention further provides a method for identifying a genetic target for an anti-nematode compound using a C. elegans strain bearing a mutation in a gene encoding a xenobiotic sensing NR (e.g., a mutation in the nhr-8, nhr-48, or daf-12 gene) as a sensitized screening background. One such method comprises the steps of: (i) contacting a population of nematodes with the compound, wherein the nematodes have a mutation in a gene encoding a xenobiotic sensing nuclear receptor, and wherein contacting a nematode with the compound causes a detectable phenotype in the nematode; (ii) mutagenizing the population of nematodes; and (iii) identifying a mutant that is resistant to the compound, wherein the resistance is manifested by the absence of the detectable phenotype in the mutant. The method can further include the step of cloning the gene that is mutated in the mutant identified in the identifying step, thereby identifying a genetic target for the compound. Alternatively, the method can be performed using nematodes that have been sensitized by contacting them with a compound that modulates (e.g., antagonizes) a xenobiotic sensing NR. It is to be understood that although many of the methods described herein refer to “a nematode”, these methods are often in practice carried out using populations of nematodes, and the invention encompasses such methods. Thus a number of the methods are useful for high throughput screening, and such screening is within the scope of the invention.

### BRIEF DESCRIPTION OF THE DRAWING

FIG. 1 is a sequence alignment showing sequence conservation of the ligand binding domain among the VDR/PXR/CAR (NR1I/J) subfamily members.

FIG. 2 is a neighbor-joining tree of selected conserved NR sequences.

FIG. 3 shows a Northern blot analysis of nhr-8 expression.

FIG. 4A is a schematic illustrating an nhr-8::GFP transgene.

FIG. 4B is a differential interference contrast micrograph of a transgenic larva bearing the nhr-8::GFP transgene and a wild type larva.

FIG. 4C is an epifluorescence micrograph of the larva in FIG. 4B.

FIG. 5 is a schematic illustrating portions of the nhr-8 gene that are deleted in the nhr8(ok186) mutant.

FIG. 5B shows a Northern blot analysis demonstrating that a truncated RNA is produced in the nhr8(ok186) mutant.

FIG. 6A shows colchicine sensitivity of wild type (N2), nhr-8(ok186), pgp-3(pk18), and nhr-8(ok186); pgp-3(pk18) worms.

FIG. 6B shows chloroquine sensitivity of wild type (N2), nhr-8(ok186), pgp-3(pk18), and nhr-8(ok186); pgp-3(pk18) worms.

FIG. 7 shows colchicine sensitivity of wild type (N2), nhr-8(ok186), pgp-3(pk18), and nhr-8(RNAi) worms.

FIG. 8 shows sensitivity to fast killing by P. aeruginosa of wild type (N2), nhr-8(ok186), and pgp-3(pk18) worms.

FIG. 9 presents a schematic illustrating nuclear receptor structure and function.

FIG. 10 presents a tree showing relationships among nuclear receptors.

FIG. 11 summarizes a primer design strategy for the amplification of a gene encoding a parasitic nematode homolog of the C. elegans NR NHR-25.

FIG. 12 presents a schematic illustrating the structure of an NR protein chimera according to certain embodiments of the invention.

FIG. 13 is a graph showing the dose response of wild-type (N2) and nhr-8(ok186) mutant animals to primaquine.

FIG. 14 is a graph showing the dose response of wild-type (N2) and nhr-8(ok186) mutant animals to atrazine.

FIG. 15 shows results of RT-PCR analysis of induction of CYP expression in response to beta-naphthaflavone or chloroquine in both N2 and nhr-8(ok186) animals.

### BRIEF DESCRIPTION OF THE TABLES

Table 1 shows C. elegans members of broadly conserved NR subfamilies.

Table 2 shows nuclear receptors from nematodes other than C. elegans and indicates members of broadly conserved subfamilies.

Table 2a is an updated version of Table 2.

Table 3 shows nuclear receptors from nematodes other than C. elegans and indicates numbers of divergent NR sequences.

Table 3a is an updated version of Table 3.

Table 4 provides a listing of various parasitic nematode orders and genera along with host name and common names of the worm or associated disease.

Table 5 lists forward primers used for semi-quantitative RT-PCR to detect mRNA transcribed from various cytochrome P450 genes.

Table 6 lists reverse primers used for semi-quantitative RT-PCR to detect mRNA transcribed from various cytochrome P450 genes, corresponding to the forward primers listed in Table 5.

### Definitions

Agonist: As used herein, the term “agonist” refers to a molecule that increases or prolongs the duration of the effect or activity of a polypeptide or a nucleic acid. Agonists may include proteins, nucleic acids, carbohydrates, lipids, small molecules, ions, or any other molecules that modulate the effect of the polypeptide or nucleic acid. An agonist may be a direct agonist, in which case it is a molecule that exerts its effect by binding to the polypeptide or nucleic acid, or an indirect agonist, in which case it exerts its effect via a mechanism other than binding to the polypeptide or nucleic acid (e.g., by altering expression or stability of the polypeptide or nucleic acid, by altering the expression or activity of a target of the polypeptide or nucleic acid, by interacting with an intermediate in a pathway involving the polypeptide or nucleic acid, etc.)

Antagonist: As used herein, the term “antagonist” refers to a molecule that decreases or reduces the duration of the effect or activity of a polypeptide or a nucleic acid. Antagonists may include proteins, nucleic acids, carbohydrates, or any other molecules that modulate the effect of the polypeptide or nucleic acid. An antagonist may be a direct antagonist, in which case it is a molecule that exerts its effect by binding to the polypeptide or nucleic acid, or an indirect antagonist, in which case it exerts its effect via a mechanism other than binding to the polypeptide or nucleic acid (e.g., by altering expression or stability of the polypeptide or nucleic acid, by altering the expression or activity of a target of the polypeptide or nucleic acid, by interacting with an intermediate in a pathway involving the polypeptide or nucleic acid, etc.). Note that the terms “agonist“ and “antagonist” are not mutually exclusive. Thus a compound may be an agonist under some conditions but may be considered an antagonist under other conditions, e.g., if it competes for binding to a target molecule with a compound that is a strong agonist of that target molecule.

Gene: For the purposes of the present invention, the term “gene” has its meaning as understood in the art. However, it will be appreciated by those of ordinary skill in the art that the term “gene” has a variety of meanings in the art, some of which include gene regulatory sequences (e.g., promoters, enhancers, etc.) and/or intron sequences, and others of which are limited to coding sequences. In general, the appropriate meaning will be evident from the context. It will further be appreciated that definitions of “gene” include references to nucleic acids that do not encode proteins but rather encode functional RNA molecules such as tRNAs. For the purpose of clarity we note that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a nucleic acid including a protein coding region.

Gene product or expression product: A gene product or expression product is, in general, an RNA transcribed from the gene or a polypeptide encoded by an RNA transcribed from the gene.

Indicator of nematode well-being: As used herein the term “indicator of nematode well-being refers to a phenotype that is characteristic of a nematode or a population of nematodes that is living normally under standard laboratory conditions as described, for example, in Lewis, J. and Fleming, J., “Basic Culture Methods”, pp. 187-204 in Methods in Cell Biology, Epstein, H. and Shakes, D., (eds.), Vol. 48, Academic Press, San Diego, 1995, and references therein. Under such conditions nematodes follow a well-characterized pattern of development, growth, and reproduction and display behaviors such as feeding, movement, defecation, mating, egg-laying, etc. In addition, they exhibit a variety of biochemical functions characteristic of these phenotypes and behaviors, e.g., synthesis of biomolecules such as DNA, RNA, protein, etc. Any detectable phenotype that is characteristic of normal nematode existence may be used as an indicator of nematode well-being. Such phenotypes include, but are not limited to: viability, growth, reproduction, development, feeding, movement, defecation, mating, and egg-laying. The indicator of nematode well-being may be a characteristic of an individual nematode or a characteristic of a population of nematodes. Thus where an indicator of nematode well-being is assessed it is not necessary to compare the indicator on a single nematode basis. Nematode strains may be selected or altered, e.g., using genetic engineering, to generate a phenotype indicative of nematode well-being or to render a particular phenotype detectable. For example, in a transgenic nematode that expresses a readily detectable protein (e.g., a fluorescent protein such as GFP), expression of the protein may be considered an indicator of nematode well-being. Expression of the protein suggests that the nematode is engaged in active biosynthetic processes. The degree to which a nematode exhibits a particular phenotype may depend upon the particular laboratory conditions under which the nematode is cultured. In general, an indicator of nematode well-being may be assessed while the nematode is grown under any selected condition and then assessed again after altering the condition, e.g., by exposing the nematode to a compound, by altering the environment or culture media, etc. Where an indicator of nematode well-being is assessed under different conditions, it is understood that the indicator need not be measured at the same time or in the same experiment but may be assessed by referring to a previous determination, a historical control, etc.

Operably linked: The term “operably linked” refers to a relationship between two nucleic acid sequences wherein the expression of one of the nucleic acid sequences is controlled by, regulated by, modulated by, etc. the other nucleic acid sequence. For example, a promoter is operably linked with a coding sequence if the promoter controls transcription of the coding sequence. An enhancer or an upstream activating sequence (UAS) is operably linked with a promoter if the enhancer modulates (i.e., increases or decreases) the activity of the promoter (i.e., the ability of the promoter to direct transcription of a coding sequence to which the promoter is operably linked). The ability of the enhancer or UAS to modulate a promoter with which it is operably linked may be controlled by the binding of a regulatory protein (e.g., a transcription factor such as a nuclear receptor) to the enhancer. Preferably a nucleic acid sequence that is operably linked to a second nucleic acid sequence is covalently linked, either directly or indirectly, to such a sequence, although any effective three-dimensional association is acceptable.

Transgenic: As used herein a transgenic organism is an organism in which one or more of the cells of the organism contain a heterologous nucleic acid introduced by way of human intervention by any of a variety of techniques well known in the art. The nucleic acid is introduced into the cell directly or indirectly by introduction into a precursor of the cell by way of deliberate genetic manipulation, e.g., by microinjection, transfection, transformation, transposon-mediated transfer, DNA microparticle bombardment, electroporation, infection with a recombinant virus, etc. One of ordinary skill in the art will be able to select the appropriate technique depending upon the organism and purpose for which the transgenic organism is to be created. The term genetic manipulation does not include classical cross-breeding but is directed to the introduction of a recombinant nucleic acid molecule. The recombinant nucleic acid molecule may be integrated into a chromosome of the animal or may be extrachromosomal, e.g., it may be an extrachromosomal array, a plasmid, etc. The transgene is may be expressed in some or all of the cells of the animal. The temporal and/or spatial expression pattern of the transgene typically depends, at least in part, on sequences such as regulatory sequences in the transgene and/or the chromosomal location of the transgene in those instances in which the transgene integrates into the chromosome.

Transgene: As used herein, the term “transgene” refers to a nucleic acid sequence that is partly or entirely heterologous, i.e., foreign to the organism or cell into which it is introduced or is homologous to an endogenous gene of the organism or cell into which it is introduced but is designed to (i) alter the genome of the organism or cell (e.g., be inserted at a location different to the endogenous location or inserted so as to create a partial or complete knockout of the endogenous gene) or (ii) be located extrachromosomally. A transgene can include one or more regulatory sequences (e.g., promoter, enhancer, polyadenylation signal, splice sites, etc.). A transgene can contain both coding and noncoding sequences, including introns.

Vector: A vector, as used herein, is a nucleic acid molecule that includes sequences sufficient to direct in vivo or in vitro replication of the molecule. These may either be self-replication sequences or sequences sufficient to direct integration of the vector into another nucleic acid present in a cell (either an endogenous nucleic acid or one introduced into the cell by experimental manipulation), so that the vector sequences are replicated during replication of this nucleic acid. Preferred vectors include a cloning site, at which foreign nucleic acid molecules may be introduced. Vectors may include control sequences that have the ability to direct in vivo or in vitro expression of nucleic acid sequences introduced into the vector. Such control sequences may include, for example, transcriptional control sequences (e.g., promoters, enhancers, terminators, etc.), splicing control sequences, translational control sequences, etc. Vectors may also include a coding sequence, so that transcription and translation of sequences introduced into the vector results in production of a fusion protein.

Specific binding: As used herein, the term refers to an interaction between a target polypeptide (or, more generally, a target molecule) and a binding molecule such as an antibody, agonist, or antagonist. The interaction is typically dependent upon the presence of a particular structural feature of the target polypeptide such as an antigenic determinant or epitope recognized by the binding molecule or a binding site such as a ligand binding domain of a nuclear receptor. Specific binding may also refer to an interaction between a polypeptide (e.g., a nuclear receptor) and its target DNA sequence. In reference to antibody/epitope interactions, for example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target polypeptide versus the affinity of the binding molecule for other targets, e.g., competitors. If a binding molecule exhibits a high affinity for a target molecule that it is desired to detect and low affinity for nontarget molecules, the antibody will likely be an acceptable reagent for immunodetection purposes. Once the specificity of a binding molecule is established in one or more contexts, it may be employed in other, preferably similar, contexts without necessarily re-evaluating its specificity.

Xenobiotic: As used herein the term xenobiotic refers to any compound that is not naturally found in or produced by a particular species, or a metabolite of such a product, e.g., a foreign compound such as an insecticide, drug, etc., introduced from the exterior. In addition to manmade compounds such as certain insecticides and drugs, xenobiotics can include compounds produced by organisms other than the particular species in question, e.g., nematodes. This could include compounds from such sources as plants, fungi, bacteria. Such compounds could be produced either prior to or after the nematode ingests or contacts the plant, fungus, or bacterium.

### DETAILED DESCRIPTION OF CERTAIN EMBODIMENTS

The present invention involves the use of the free-living nematode Caenorhabditis elegans (C. elegans) as a model system for the identification of anti-nematode agents. The invention encompasses the recognition that members of the nuclear receptor (NR) superfamily of transcription factors in parasitic nematodes represent promising targets for the development of new agents for nematode control. The invention further encompasses the recognition that NR family members in parasitic nematodes and the signal transduction pathways in which they function have counterparts in C. elegans. The invention exploits the shared biology between C. elegans and parasitic nematode species and draws upon the wealth of information and experimental techniques available for the study of C. elegans to develop methods of screening for compounds that possess anti-nematode activity.

The invention encompasses the discovery that nematodes, e.g., C. elegans contain homologs (e.g., paralogs and/or orthologs) of mammalian xenobiotic sensing NRs and that there is conservation between the xenobiotic sensing function and response in mammals and that in nematodes, e.g., C. elegans. For example, as described herein, the C. elegans NR NHR-8 is expressed in the nematode gut and is involved in the response of C. elegans to xenobiotics, and reduction in the activity of the NR encoded by nhr-8 either by mutation or by RNA-mediated genetic interference (RNAi) results in increased sensitivity of C. elegans to toxins (see Examples 4, 5, and 7). Therefore, agents that decrease or eliminate the activity of NHR-8, such as antagonists of the NHR-8 protein, are useful to increase the efficacy of other anti-nematode compounds, e.g., by crippling a portion of the nematode's toxin defense system and thereby making the nematode more sensitive to deleterious compounds. Agonists are also potentially useful, e.g., in certain cases in which the active form of an anti-nematode compound is actually a metabolite of the applied compound (as is true of the insecticide indoxacarb). In such cases an NR agonist could be used to increase the efficacy of the applied compound by hyperstimulating expression of the metabolic enzymes required for activation of the compound.

The invention further encompasses the realization that a C. elegans strain bearing a mutation in a gene encoding a xenobiotic sensing NR such that the function of the NR is impaired, provides a sensitized background for screening for anti-nematode agents or for identifying the target(s) of known anti-nematode compounds. For example, a C. elegans strain bearing a mutation in the nhr-8 gene, such that the amount of functional activity of the NHR-8 protein is reduced and/or impaired, provides a sensitized background for screening for anti-nematode agents or for identifying the target(s) of known anti-nematode compounds. In addition, the invention includes the use of related nematode NRs, e.g., the NR encoded by the C. elegans nhr-48 gene and homologs thereof, for similar purposes.

In order that the invention may be better understood, the following section discusses certain aspects of the life cycles and characteristics of parasitic nematodes. Subsequent sections offer an overview of the use of C. elegans as a model system in the context of the present invention, discuss the structure and features of nuclear receptors, and describe bioinformatics approaches used in the identification and analysis of NRs. Particular aspects of the invention are discussed in the remaining sections.

A. Parasitic Nematodes and their Life Cycles

The life cycles of both free-living and parasitic nematodes involve a series of larval stages, generally four designated L1 through L4, separated by molts during which feeding, growth, development, and activity cease, and a new external cuticle is deposited. The old cuticle is then shed, and the animal resumes feeding, growth, and other activities. Following the L2 stage, many parasitic nematodes arrest development until infection of the host occurs. The developmental arrest can persist for prolonged and variable periods of time, during which the animal exhibits phenotypes specialized for infectivity. For example, during the infective L3 stage, animals are highly motile and environmentally resistant. The infective L3 stage resembles the developmentally arrested dauer stage of C. elegans

As indicated in the Background, a wide variety of plant and animal parasitic nematodes exist. As the term is used herein, parasitic nematodes are distinguished in that they must (1) undergo at least part of their life cycle (e.g., development, growth, and/or reproduction) within their respective host(s) and/or (2) obtain a significant proportion of their nutrition by feeding on a living organism to which they remain attached during at least a portion of their life cycle while remaining largely outside the organism and not internalizing the organism. This latter mode of parasitism is to be distinguished from the consumption of living organisms (e.g., bacteria) or the saprophytic behavior characteristic of free-living nematodes. Plant parasitic nematodes may be broadly classified as ectoparasites, which remain outside the plant host although they may insert part of their body (e.g., feeding stylet and/or feeding stylet and part of the body itself) into the plant, and endoparasites, which enter the plant (typically via the root) and may migrate therein. Animal parasitic nematodes (which include nematode parasites of humans) typically spend at least a portion of their life cycle within the body of the host. Detailed information regarding many of the known plant and animal parasites of importance may be found in Perry, R. N. and Wright, D. J. (eds.), The Physiology and Biochemistry of Free-living and Plant-parasitic Nematodes, CABI Publishing, New York, 1998, and in Anderson, R. C., Nematode Parasites of Vertebrates, 2^{nd} ed., CABI Publishing, New York, 2000, respectively, and references listed in these volumes.

B. C. elegans as a Model System for Parasitic Nematodes

Molecular genetic methods (such as gene knockouts, transgenic animals, etc.) can uncover the biological function of individual genes and proteins in an organism, information that can form the foundation for developing target-based compound discovery screens. However, at present these methods are difficult to perform in parasitic nematodes. In contrast, such procedures can be performed in a straightforward manner in C. elegans. Furthermore, the complicated life cycle of many parasitic nematodes and their need for a suitable plant or animal host makes it inconvenient to propagate them in the laboratory.

Over the past twenty-five years extensive study of C. elegans has led to its recognition as a pre-eminent “small metazoan” model for understanding the development, neurobiology, and genetics of more complex animals (Riddle D. L., Blumenthal T., Meyer B. J., Priess J. R., C. elegans II, Cold Spring Harbor: Cold Spring Harbor Laboratory Press, 1997; Wood W. B., The Nematode Caenorhabditis elegans, Cold Spring Harbor: Cold Spring Harbor Laboratory Press, 1988). The strengths of the C. elegans model include: rapid reproduction (four days), a transparent body that allows observation and identification of each cell in live animals, a completely delineated developmental cell lineage and neural anatomy, and the first fully sequenced genome of a multicellular organism. Rapid and powerful techniques for generating and isolating genetic mutants, mapping mutations, cloning genes, and creating transgenic C. elegans are all well known in the art and can be accomplished in a much shorter time period (weeks to months) than in most other metazoan model systems. Moreover, C. elegans is readily cultured both on Petri dishes containing E. coli as food and in liquid culture. As described further below, this last property makes this nematode eminently adaptable for arraying and assaying in microtiter plates and suitable for high-throughput chemical screening against thousands of compounds simultaneously (Link, E. M., Hardiman G., Sluder, A., Johnson, Carl D., and Liu L. X., Therapeutic target discovery using Caenorhabditis elegans, Pharmacogenomics 1(2), 2000).

In the last decade, numerous avenues of research into the fundamental mechanisms of organismal growth, development, and behavior utilizing different model organisms have converged, in no small part due to rapidly accumulating DNA sequence information and computational biology resources such as those developed by the C. elegans Sequencing Consortium (C. elegans Sequencing Consortium T, Genome sequence of the nematode C. elegans: a platform for investigating biology, Science 282, 2012-2018, 1998). For example, the majority of human genes (as extrapolated from the set of positionally cloned human disease genes) have C. elegans homologs defined by standard sequence comparison algorithms (Bassett D. E., Jr. et al., Genome cross-referencing and XREFdb: implications for the identification and analysis of genes mutated in human disease, Nat Genet 15, 339-344, 1997). In fact, individual proteins, cellular structures and regulatory pathways are often conserved among evolutionarily divergent species such as yeast, fruit flies, nematodes, mice, and humans. Thus, cross-genomic studies of such fundamental biological processes as the cell cycle, the ras pathway, and apoptosis have demonstrated that structurally homologous genes are often functionally homologous as well, i.e. perform the same genetic and biochemical function across species. For this reason, targets and pathways that are conserved between C. elegans and other organisms can be used, for example, as a model for human disease (Ahringer J., Turn to the worm. Curr Opin Genetics Dev 7, 410-415, 1997) or as a platform for pharmaceutical discovery (Gil E., Link E. M., Liu L. X., Johnson C. D., Lees J. A., Regulation of the insulin-like developmental pathway of Caenorhabditis elegans by a homolog of the PTEN tumor suppressor gene, Proc Natl Acad Sci USA 96, 2925-2930, 1999). As described herein such targets and pathways can be used as the basis for screens for the identification of new anti-nematode compounds.

While many C. elegans genes are homologous to those of humans and other vertebrates, C. elegans genes are much more similar to those of parasitic nematode species as judged by comparison with sequences available in sequence databases. Beyond such sequence similarities, basic mechanisms controlling development and behavior are likely to be shared between C. elegans and parasitic nematodes, in spite of life histories and survival strategies that superficially appear very different (Riddle D. L., Georgi L. L., Advances in research on Caenorhabditis elegans: application to plant parasitic nematodes, Annu Rev Phytopathol 28, 247-269, 1990). For example, the dauer larva of C. elegans, an alternative third-stage larva specialized for dispersal and long-term survival, bears numerous parallels to the obligatory infective stage larvae of nematode parasites (Hotez P, Hawdon J., Schad G. A., Hookworm larval infectivity, arrest and amphiparatenesis: the Caenorhabditis elegans daf-c paradigm. Parasitology Today 9, 23-26, 1993). Entry into the dauer stage can be triggered by crowding and starvation, and the genetic pathways underlying entry into and recovery from dauer have been extensively studied. The C. elegans genome sequence also contains a large number of apparently nematode-specific gene families (Sonnhammer E. L., Durbin R., Analysis of protein domain families in Caenorhabditis elegans, Genomics 46, 200-216, 1997), providing a rich source of potential nematode-selective biological targets for nematicide and anthelminthic discovery.

C. elegans contains five pharyngeal gland cells, three of them known collectively as g1 cells and two called g2 cells. The g1 cells are equivalent to the three pharyngeal gland cells found in plant parasitic nematodes and contain secretory vesicles (Bird, A. F. and Bird, J., 1991, referenced above). In addition, C. elegans possesses amphids, and C. elegans amphid sheath cells are believed to secrete matrix material that surrounds the neuron processes (Riddle, D. L. and Albert, P. S., Regulation of Dauer Larva Development, in C. elegans II, referenced above). In free-living nematodes, secreted proteins may include proteins unrelated to parasitism but important in general nematode physiology or development while in parasitic nematodes secreted proteins may also include proteins that play a role in parasitism.

While the many advantages of C. elegans described above make it a preferred organism for the practice of the present invention, the invention may also be practiced using a variety of other nematode species. In particular, the invention may be practiced using various free-living nematode species that can be propagated under laboratory conditions, e.g., C. briggsae, C. remanei, etc.

C. Nuclear Receptor Superfamily

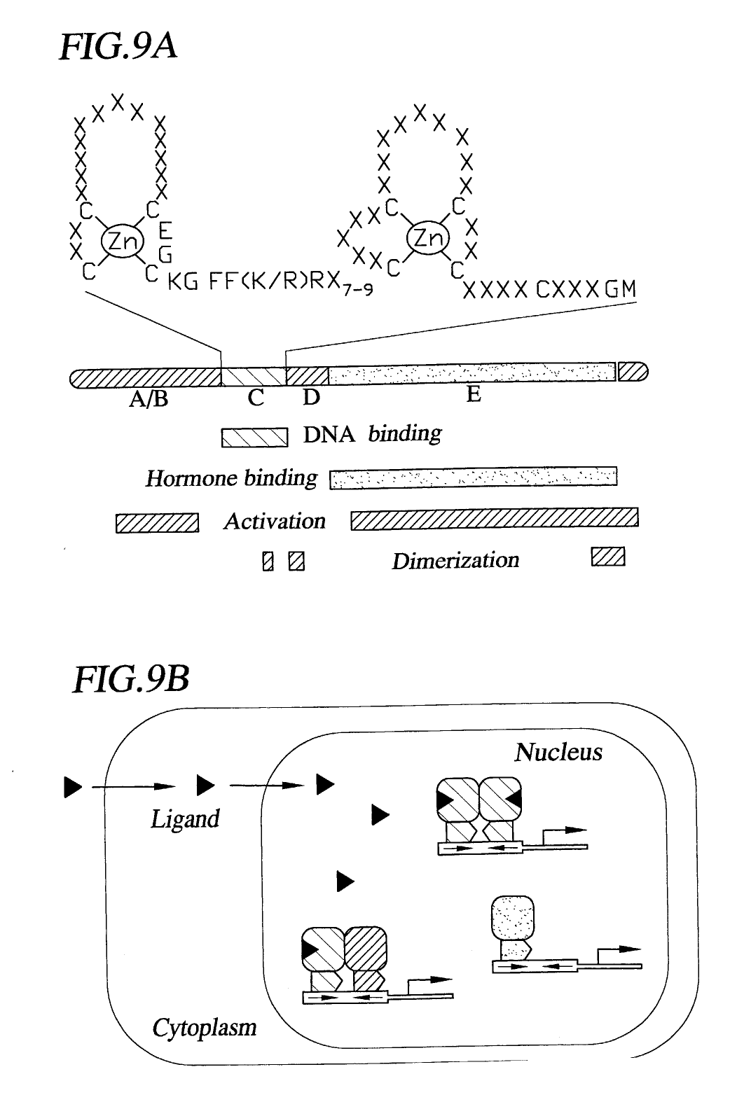

Nuclear receptors are one of the most abundant classes of transcriptional regulators (transcription factors) in metazoans. Various NRs are involved in processes as diverse as sexual differentiation, metabolic regulation, insect and amphibian metamorphosis, vertebrate limb development, and embryonic pattern formation ¹¹⁻¹³, (14,15). The defining members of the NR superfamily were first identified biochemically as receptors for mammalian steroid and thyroid hormones. As shown in FIG. 9A, NRs share a modular protein architecture characterized by two conserved core domains flanked by more variable sequences. The centrally positioned DNA binding domain (DBD) contains two Cys₂-Cys₂ zinc-coordinating modules (16). The highly conserved structural elements of this domain make it the signature motif for the superfamily. As shown in FIG. 9B, NRs can control transcription as dimers or monomers, and different NRs function preferentially in different modalities. Each NR monomer studied appears to recognize a specific hexamer DNA element. The figure illustrates the three most typical modes of activity, those of activating transcription as: (1) a ligand bound homodimer bound to a response element composed of two hexamer half-sites arranged as an inverted repeat; (2) a ligand-bound homodimer bound to a direct repeat response element; and (3) a monomer bound to a single extended DNA element. Some NRs are also found in the cytoplasm under certain conditions, and other modes of activity are possible.

A unified nomenclature system for the NR superfamily has been described and is generally accepted in the field (Nuclear Receptors Committee, A unified nomenclature system for the nuclear receptor subfamily, Cell, 97, 1-2, 1999; Also see the Web site having URL www.ens-lyon.fr/LBMC/laudet/nomenc.html and references therein for further detail). This nomenclature system is used herein. The system divides NRs into six subfamilies (NR1-NR6) based on protein sequence. The subfamilies are further divided into groups.

In many cases the ability of a nuclear receptor to regulate gene expression is modulated by binding of a small molecule ligand to a domain positioned C-terminal to the DBD. Known ligands include steroid, thryroid, and retinoid hormones⁷ as well as metabolic intermediates⁹ and xenobiotics¹⁰. The ligand-binding domain (LBD) also participates in receptor homo- and heterodimerization, and contributes to transcriptional regulation. Though less highly conserved than the DBD, the LBDs of the classical nuclear receptors also contain regions of similarity (17). In addition, some NRs have an extended carboxy-terminal domain of unknown function⁵. Comparative analysis based on the core DBD and LBD sequences defines six major subfamilies among the non-nematode NRs⁶.

The GenBank database currently contains over 77 cloned NR sequences from more than 60 species. The non-nematode sequences define at least 70 distinct NRs (18), and ligands or candidate activators have been reported for 26 of these (19-21). However, the majority of NRs remain orphan receptors for which the specific ligands, if any exist, have not yet been identified. Genetic studies reveal roles for orphan NRs in a similarly wide spectrum of processes to those in which NRs with identified ligands play a role^{12, 13}

In mammals, NR agonists and antagonists derived from steroid and terpenoid hormones that are known NR ligands have proven to be effective drugs since they easily cross cell membranes and often can be administered orally. For example, birth control pills employ estrogens and progesterones, and synthetic glucocorticoids are potent anti-inflammatory agents. More recently, synthetic retinoids have been identified as lead compounds for the treatment and prevention of a variety of cancers (22,23). The peroxisome proliferator activated receptor PPARγ has been found to be a direct target of the thiazolidinedione class of FDA-approved antidiabetic drugs (24,25). Thus many compounds that modulate NR function have been developed successfully into drugs.

D. Role of Nuclear Receptors in Sensing and Metabolism of Xenobiotics

In vertebrates, exogenous xenobiotic and pharmacologic compounds can regulate gene expression by a similar mechanism to that by which endocrine signals (e.g., hormones) act. Such compounds bind to and modulate the activity of specific xenobiotic sensing NRs, leading to alteration in expression of target genes [2-4]. Thus certain NRs play important roles in pathways leading to the metabolism and resulting detoxification of a wide variety of endogenous compounds (e.g., steroid hormones) and/or xenobiotic (i.e., foreign) compounds in vertebrates. Much of the metabolism is performed by cytochrome P-450 (CYP-450) enzymes, many of which are inducible by a large number of different compounds and have broad substrate specificity. It has recently been discovered that this induction is mediated, at the transcriptional level, by NRs which, in response to a diversity of compounds, bind to and induce transcription from transcriptional response elements associated with CYP-450 genes. For example, the human nuclear receptor known as PXR or as SXR (steroid and xenobiotic receptor) has been shown to respond to a diverse array of steroids and drugs such as RU-486, phenobarbital, many C21 sterols and pregnanes, rifampicin and nifedipine. Treatment with such agents triggers binding of PXR/SXR to response elements within the promoters of various CYP-450 genes, which leads to increased levels of transcription [6]. Activation of the expression of CYPs by SXR/PXR leads to increased biotransformation of ingested xenochemicals as well as endogenous metabolites [5-8]. Likewise, human CAR and chicken CXR respond to xenobiotic compounds by regulating the expression of CYP-450 genes [4-9]. A nuclear receptor that responds to the presence of a xenobiotic by playing a role in a pathway (e.g., a physiologic or biochemical pathway) that affects the metabolism or biotransformation of the compound is referred to herein as a xenobiotic sensing nuclear receptor.

All known CYP-450 regulatory NRs belong to the same nuclear receptor gene subfamily, NR1. Those NRs identified to date as xenobiotic sensing NRs fall into either the NR1I or NR1J groups (subsets of the NR1 subfamily), although it is possible that future work will reveal others falling into different groups within the NR1 subfamily or within a different subfamily. The NR1I and NR1J groups consistently behave as “sister groups” in all trees, consistent with the close relationship between members of these two groups. Therefore the NR1I and NR1J groups will be referred to collectively herein as the NR1I/J group, i.e., a member of either the NR1I or NR1J groups is considered a member of the NR1I/J group. Membership in the NR1I/J group is one criterion on which classification of an NR as a xenobiotic sensing NR may be based although it is not necessarily the case that all members of the NR1I/J group function as xenobiotic sensing NRs or that no xenobiotic sensing NRs fall outside this group.

E. Bioinformatics Useful in Identification and Analysis of Nuclear Receptors

Genes within a large family such as that of the nuclear receptors can be related in several ways. First, two genes in two different species that derive directly from a single gene in the last common ancestor of the two species are orthologs. Second, gene duplication events within a species produce multiple homologous genes known as paralogs. If the duplication occurred in a common ancestor of two species, each paralog within one species will have an ortholog in the other (in the absence of any gene loss). However, if one or more duplication events occurred after the evolutionary divergence of two species, there will not be definitive one-to-one orthologous relationships between genes in the two species. The term “paralogs” is used herein to refer only to those related genes within one species that as a group correspond to a single ortholog or orthologous group in another species. Broader relationships within the NR gene family will be indicated by less restrictive terms such as group or subfamily (as defined for the NR nomenclature at the Web site having URL www.ens-lyon.fr/LBMC/laudet/nomenc.html), thus limiting use of the less precise “homolog”.

Unambiguous identification of orthologs may not always be straightforward, especially when not all members of a gene family are known for a species of interest. There are several approaches that can be used to identify likely orthologous gene pairs. The least time-intensive strategy is to perform reciprocal BLAST searches, for example, using tools and data available at the Web site having URL www.ncbi.nlm.nih.gov/BLAST/. If two gene sequences are orthologous, each should identify the other as the most closely related sequence in the other species. Orthologs should share domain similarity along their entire lengths. A more stringent strategy uses comprehensive molecular phylogenetic analyses of all available members of a gene family to identify apparent orthologs. Ortholog groupings should be supported with significant confidence values (minimally ≧50%, preferably ≧90%) when evaluated by statistical means such as bootstrap analysis, and ideally the groupings will be corroborated by more than one comparison method (e.g., both distance methods such as neighbor-joining and parsimony analysis). (See, e.g., Burglin, T. R., A comprehensive classification of homeobox genes. pp. 25-71. In: Guidebook to the Homeobox Genes. Duboule, D. (Ed.), Oxford University Press, Oxford, UK, 1994 and Laudet, V. (1997) Evolution of the nuclear receptor superfamily: early diversification from an ancestral orphan receptor. Journal of Molecular Endocrinology 19, 207-226, 1997, for comprehensive examples of molecular phylogenetic analyses of gene families.) Conservation of surrounding gene order on the chromosome (i.e., synteny) can also provide support for the assignment of orthology between two genes. Finally, complementation of a loss-of-function mutation in one species by transgenic introduction of the candidate ortholog from the other species can demonstrate the conservation of key functional characteristics, though this alone is not sufficient to determine orthology as core biochemical characteristics may be preserved in multiple gene family members. To the extent that available data and reagents permit, all of these strategies except transgenic complementation have been used herein to define orthologous relationships for the nematode NRs (methods employed are described in fuller detail in Sluder et al., The nuclear receptor superfamily has undergone extensive proliferation and diversification in nematodes. Genome Research 9, 103-120, 1999).

F. Nuclear Receptors in C. elegans and Other Nematodes

The C. elegans genome sequence reveals at least 270 predicted NR genes (15), 5-fold more than the number reported from any other species. NRs are the most abundant class of predicted transcriptional regulators encoded in the C. elegans genome. Numerous apparent gene duplications have contributed to this abundance 15, and some of the C. elegans NR sequences might be nonfunctional pseudogenes. However, several observations suggest that the majority of the predicted NR genes are functional. First, expressed sequence tags (ESTs) confirm mRNA expression for 79 (or 29%) of the 270 predicted NR genes. Second, in a study of NR gene promoters fused to the green fluorescent protein (GFP) reporter, expression was observed in transgenic animals for 21 of 28 NR genes surveyed, suggesting that most predicted C. elegans NR genes are functional transcription units 16. Despite this demonstration of promoter function, only eight of the 21 expressed reporter constructs have corresponding ESTs. Thus EST representation provides an underestimate of expressed NR genes, suggesting that many of these genes are transcribed at low levels or in only a few cells. Finally, the majority of available NR cDNA and EST sequences represent spliced transcripts and do not reveal inappropriate stop codons as would appear for pseudogenes encoding nonfunctional mRNAs, further suggesting that most of the C. elegans NR sequences are likely to represent functional genes.

Among the metazoans for which genome sequences have been essentially completed, such extravagance in NR genes is unique to C. elegans. The 270 predicted nematode NR genes vastly outnumber those in the genomes of either Drosophila (21 genes17) or humans (48 genes, Maglich, J. M., A. Sluder, X. Guan, Y. Shi, D. D. McKee, K. Carrick, K. Kamdar, T. M. Willson, and J. T. Moore. (2001) Comparison of complete nuclear receptor sets from the human, C. elegans, and Drosophila genomes. Genome Biology, 2: 0029.1-0029.7). Fifteen of the C. elegans NR genes have apparent orthologs or close homologs in insects and vertebrates (See Table 1). These fifteen can be placed within five of the six major NR subfamilies defined by phylogenetic analysis of vertebrate NRs, consistent with the proposed early metazoan origin of these NR families ⁶. The remaining 255 C. elegans NR genes are not placed in these subfamilies by the analyses performed thus far 15 and reveal a spectrum of NR variety far exceeding that known before the sequencing of the C. elegans genome. More robust comparative sequence analyses will be needed to determine if these 255 NRs define new major NR subfamilies or represent significantly diverged groups (as defined¹⁸) within one or more of the six currently recognized NR subfamilies.

Analysis of the C. elegans NR genes based on the DNA binding domain motif revealed that several NR classes conserved in both vertebrates and insects are also represented among the nematode genes (15), consistent with the existence of ancient NR classes shared among most, and perhaps all, metazoans (26). However, the vast majority of the nematode NR sequences define at least 16 NR classes distinct from those currently known in other phyla, and reveal a previously unobserved diversity within the NR superfamily. These apparent nematode-specific sequences will be referred to herein as divergent nematode NRs, whereas those NRs in classes shared among multiple phyla will be referred to as conserved NRs. Anti-nematode compounds selective for nematode but not mammalian members of conserved NR classes would minimize the potential for toxicity to mammals. The feasibility of developing selective compounds has been demonstrated by the identification of compounds selective for particular members of groups of closely related mammalian NRs (27,28).

TABLE 1
C. elegans members of broadly conserved NR subfamilies^{a}

| | | Drosophila | |
|---|---|---|---|
| NR group^{b} | C. elegans^{c} | ortholog (paralog)^{d} | Human paralogs |
| NR1D/E/G | NHR-85 | E75 (E78)* | Reverbα, β |
| | SEX-1 (NR1G1) | | |
| NR1F | NHR-23 (NR1F4) | HR3* | RORα, β, γ |
| NR1I/J | NHR-8 | HR96 | PXR, CAR, VDR |
| | DAF-12 | | |
| | NHR-48 | | |
| NR2A | NHR-64 | HNF4 | HNF4α, γ |
| | NHR-69 | | |
| NR2C/D | NHR-41 | HR78* | TR2, TR4 |
| NR2E | NHR-67 | TLL | TLX |
| | FAX-1 (NR2E5) | CG16801^{e} | PNR |
| NR2F | UNC-55 | Seven-up | COUP-TF1, |
| | | COUP-TF2, | |
| | | EAR2 | |
| NR4A | NHR-6 (NR4A4) | HR38* | NGFI-Bα, β, γ |
| NR5A | NHR-25 | FTZ-F1* | SF1, LRH1 |
| NR6? | NHR-91 | HR4 | GCNF? |

^{a}The relationships of C. elegans NR sequences to those from other phyla were assessed as described¹⁵. Orthology was assigned only for NR pairs for which an unambiguous correspondence could be ascribed.
^{b}The major NR subfamilies NR1-NR6 are subdivided into groups designated by capital letters. Additional information regarding NR subfamily assignments and group designations is given¹⁸ and at the Web site having URL www.enslyon.fr/LBMC/laudet/nomenc.html.
^{c}These C. elegans members of conserved NR subfamilies have been submitted for assignment of official NR designations, but not all assignments have yet been completed. Those assignments that are known are indicated in parentheses.
^{d}The Drosophila NRs involved in the ecdysone-response cascade¹³ are indicated with *.
^{e}Predicted gene as named in FlyBase (available at the Web site having URL flybase.bio.indiana.edu).

The nematode species that are known to have NRs span a wide range of nematode phylogeny and lifestyles (free-living, animal parasitic, and plant parasitic)¹⁹, providing an initial glimpse of the scope of the NR superfamily among nematodes. NR sequences have been identified by homology cloning from the human and animal parasitic nematodes Brugia malayi (C. Maina, pers. comm.), Brugia pahangi (29), Onchocerca volvulus (30,31), Dirofilaria immitis (C. Maina, pers. comm.), and Strongyloides stercoralis (32). By searching public databases containing expressed sequence tags (ESTs), the inventors have identified cDNAs encoding additional NRs from the animal parasitic nematodes Brugia malayi, Strongyloides stercoralis, Haemonchus contortus, and Trichinella spiralis; the plant parasitic nematodes Meloidogyne incognita and Heterodera glycines; and the free-living species Pristionchus pacificus and Zeldia punctata. (See Tables 2 and 3). The sequences define at least 27 distinct genes (as of December 2000), some represented by orthologs in more than one species. Based on the limited amount of sequence information currently available for nematodes other than C. elegans, it appears that the large number and diversity of NRs found in C. elegans is a general feature of nematodes, including important parasitic nematode species. On the basis of the available genome sequence, the free-living Caenorhabditis briggsae appears likely to have as many NR genes as its sibling species C. elegans¹⁵. Currently, the most extensive EST collection for a nematode other than C. elegans is that assembled for the filarial parasite Brugia malayi²⁰, with 22,932 ESTs as of December 2000. The B. malayi genome size and gene number are estimated to be very similar to those of C. elegans²⁰. By making some assumptions about the ratios of clones screened to ESTs reported for the C. elegans and B. malayi projects and assuming that NR genes are similarly underrepresented in ESTs from both species, the inventors estimate that the number of B. malayi NR genes is similar to that found in C. elegans. These estimates for C. briggsae and B. malayi indicate that NR abundance could be a general feature of nematodes.

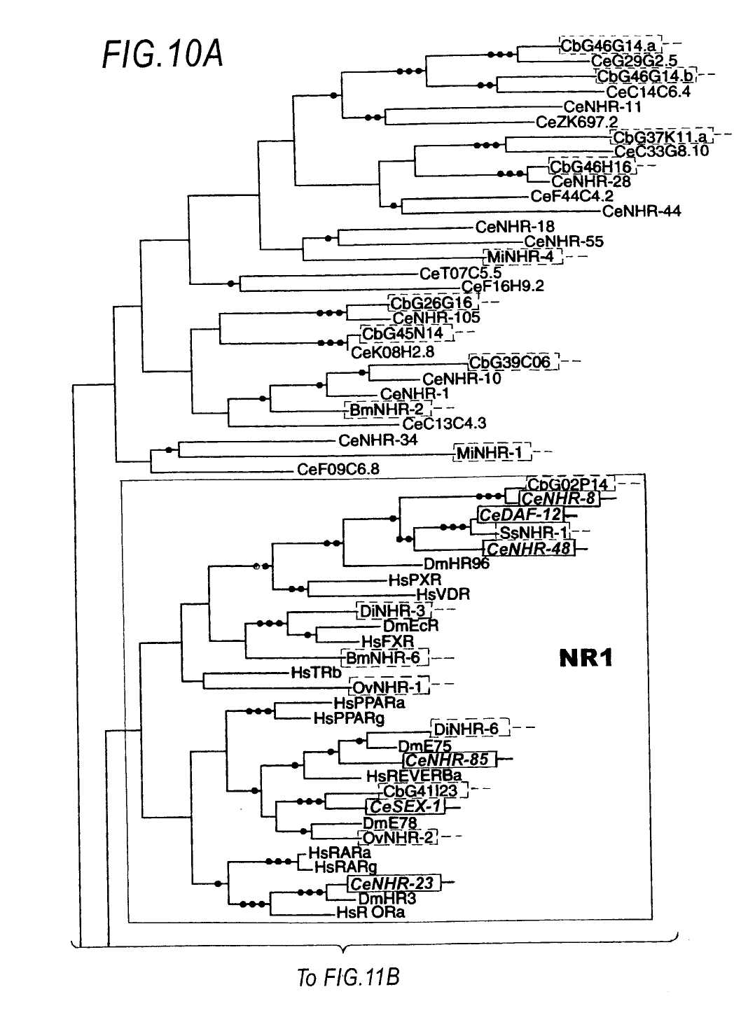

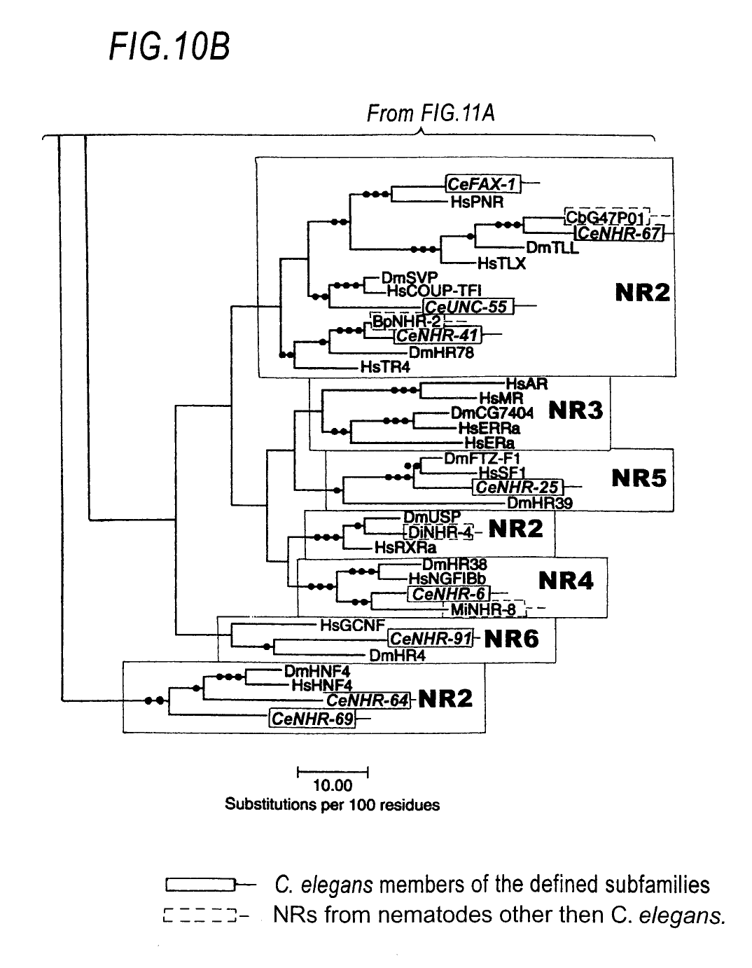

NR diversity also appears to be a general characteristic of nematodes. FIG. 10 presents a tree illustrating relationships between various nematode and non-nematode NRs. This neighbor-joining tree of NR DNA-binding domain sequences was generated using the paupsearch feature of the GCG 10.1 program package (1000 bootstrap replicates, with midpoint rooting). Significant bootstrap support values are indicated by dots on the appropriate branches: (.) 50-79%; (. .) 80-94%; ( . . . ) ≧95%. Sequences included were selected to represent the six recognized NR subfamilies (see the Web site having URL www.ens-lyon.fr/LBMC/laudet/nomenc.html) as well as each of the major groupings of divergent C. elegans NRs evident in a larger tree containing all the nematode sequences. The boxes indicate the six major NR subfamilies; although the integrity of subfamily 2 was not preserved by this limited analysis, all major groups within the subfamily are maintained. C. elegans members of the defined subfamilies are boxed and indicated with the prefix Ce. The other boxes highlight NRs from nematodes other than C. elegans. The first two letters of sequence names indicate the species of origin: Brugia malayi (Bm); Brugia pahangi (Bp); Caenorhabditis briggsae (Cb); Caenorhabditis elegans (Ce); Dirofilaris immitis (Di); Drosophila melanogaster (Dm); Meloidogyne incognita (Mi); Onchocerca volvulus (Ov); Homo sapiens (Hs); Strongyloides stercoralis (Ss).

Many of the NR genes known from nematodes other than C. elegans and C. briggsae were identified in molecular screens for conserved NR sequences²¹⁻²³ and therefore are, not surprisingly, members of the defined NR subfamilies (Table 2, FIG. 10). Genome (see the Web site having URL genome.wustl.edu/gsc/Projects/briggsae.shtml) and EST^{20,24} sequencing projects, on the other hand, have revealed divergent NRs (Table 3, FIG. 10). Orthologs are found in the C. elegans genome for some of the NR genes from other nematodes, but not for all—even C. briggsae has some NR genes without C. elegans orthologs¹⁵. C. elegans appears to lack orthologs of EcR and Usp, the components of the insect ecdysteroid receptor¹³, although apparent orthologs of these NRs are found in filarial nematodes (G. Tzertzinis, K. Crossgrove, C. Shea, & C. Maina, in preparation). The most parsimonius explanation for this difference is that these genes have been lost (or dramatically diverged) in the C. elegans evolutionary lineage. This loss suggests that not all NRs represented in both other metazoans and in parasitic nematode species are represented in C. elegans. Thus identification of additional parasitic nematode NRs based upon comparisons with, for example, vertebrate and/or insect NRs is likely to reveal the existence of parasitic nematode NRs that do not necessarily have paralogs or orthologs in C. elegans. Diversity among the divergent nematode NRs suggests that addition of new NR subtypes—or rapid evolutionary change of a subset of NRs—has also occurred differentially among the branches of nematode phylogeny. These observations are consistent with the hypothesis that proliferation and diversification of NR sequences have continued throughout nematode evolution, with distinct NRs contributing to specific adaptations for particular lifestyles¹⁵, e.g., parasitic lifestyles.

TABLE 2
Nuclear receptors from nematodes other than C. elegans: Members of
broadly conserved NR subfamilies^{a}

| NR | Nematode NR | C. elegans | Drosophila | Human | |
|---|---|---|---|---|---|
| group | genes^{b} | ortholog | ortholog^{d} | paralogs | References |
| NR1D | Bmnhr-8, Dinhr- | nhr-85 | E75 | Reverbα, β | 20 |
| | 6^{c} | | | | |
| NR1E | Bmnhr-7, Dinhr- | | E78 | | 20,23 |
| | 7^{c}, Ovnhr-2 | | | | |
| NR1G | CbG41I23 | sex-1 | | | |
| NR1H | Bmnhr-3^{c}, | | EcR | FXR, LXRα, β | 20 |
| | Dinhr-3^{c} | | | | |
| | Bmnhr-6 | | | | |
| NR1I/J | CbG02P14 | nhr-8 | HR96 | PXR, CAR, VDR | 35 |
| | Dinhr-5^{c}, Ssnhr-1 | daf-12 | | | |
| NR1K | Ovnhr-1 | | | | 21 |
| NR2B | Bmnhr-4^{c}, | | Usp | RXRα, β, γ | |
| | Dinhr-4^{c} | | | | |
| NR2C/D | Bmnhr-5^{c}, | nhr-41 | HR78 | TR2, TR4 | 22 |
| | Bpnhr-2 | | | | |
| NR2E | CbG47P01 | nhr-67 | TLL | TLX | |
| | Dinhr-1^{c} | fax-1 | CG16801 | PNR | |
| NR4A | Bpnhr-1, Dinhr- | nhr-6 | HR38 | NGFI-Bα, β, γ | 22,24 |
| | 2^{c}, Minhr-8 | | | | |
| NR5A | CbCB015N01, | nhr-25 | FTZ-F1 | SF1, LRH1 | 24 |
| | Ssnhr-3 | | | | |
| NR6? | Ssnhr-4 | nhr-91 | HR4 | GCNF? | 24 |
| ^{a}The relationships among NR sequences were assessed as for Table 1. | | | | | |
| ^{b}C. briggsae (Cb) sequences are indicated by the name of the corresponding genomic clone (see the Web site having URL genome.wustl.edu/gsc/Projects/briggsae.shtml). For previously unnamed genes from parasitic nematodes, the inventors have assigned gene names in accordance with established conventions^{67,68} - the first two letters indicate species of origin, and the remainder of each gene name follows the C. elegans | | | | | |
| #nomenclature conventions. The species represented are Brugia malayi (Bm), Brugia pahangi (Bp), Dirofilaria immitis (Di), Meloidogyne incognita (Mi), Onchocerca volvulus (Ov), and Strongyloides stercoralis (Ss). | | | | | |

^{c}Indicated sequences are from K. Crossgrove, G. Tzertzinis, C. Shea, and C. Maina (in preparation). All other sequences are present in public databases (as of December 2000).
^{d}The Drosophila NRs involved in the ecdysone-response cascade¹³ are indicated with*.

By searching public databases containing expressed sequence tags (ESTs), the inventors have identified cDNAs encoding additional NRs from the animal parasitic nematodes Ancylostoma caninum, Ascaris suum, Brugia malayi, Parastrongyloides trichosuri, Strongyloides ratti, Strongyloides stercoralis, Haemonchus contortus, and Trichinella spiralis; the plant parasitic nematodes Meloidogyne incognita, Meloidogyne javanica, Globodera rostienchus, and Heterodera glycines; and the free-living species Pristionchus pacificus and Zeldia punctata. (See Tables 2a and 3a). The sequences define at least 49 distinct genes (as of March 2002), some represented by orthologs in more than one species.

TABLE 2a
Nuclear receptors from nematodes other than C. elegans: Members of
broadly conserved NR subfamilies^{a}

| NR | Nematode NR | C. elegans | Drosophila | Human | |
|---|---|---|---|---|---|
| group | genes^{b} | ortholog | ortholog^{d} | paralogs | References |
| NR1D | Bmnhr-8, Cbnhr- | nhr-85 | E75* | Reverbα, β | 20 |
| | 85 Dinhr-6^{c} | | | | |
| NR1E | Bmnhr-7, Dinhr- | | E78* | | 20,23 |
| | 7^{c}, Ovnhr-2 | | | | |
| NR1F | Asnhr-6, Cbnhr- | nhr-23 | HR3* | RORα, β, γ | |
| | 23, Tsnhr-2 | | | | |
| NR1G | CbG41I23 | sex-1 | | | |
| NR1H | Bmnhr-3^{c}, | | EcR* | FXR, LXRα, β | 20 |
| | Dinhr-3^{c} | | | | |
| | Bmnhr-6 | | | | |
| NR1I/J | CbG02P14 | nhr-8 | HR96 | PXR, CAR, VDR | 35 |
| | Cbnhr-48 | nhr-48 | | | |
| | Asnhr-7, Cbdaf- | daf-12 | | | |
| | 12, Dinhr-5^{c}, | | | | |
| | Ssnhr-1 | | | | |
| NR1K | Ovnhr-1 | | | | 21 |
| NR2A | Cbnhr-64, | nhr-64 | HNF4 | HNF4α, γ | |
| | Grnhr-1 | nhr-69 | | | |
| | Cbnhr-69 | | | | |
| NR2B | Asnhr-5, Bmnhr- | | Usp* | RXRα, β, γ | |
| | 4^{c}, Dinhr-4^{c} | | | | |
| NR2C/D | Bmnhr-5^{c}, | nhr-41 | HR78* | TR2, TR4 | 22 |
| | Bpnhr-2, Cbnhr- | | | | |
| | 41, Ssnhr-7, | | | | |
| | Tsnhr-3 | | | | |
| NR2E | CbG47P01 | nhr-67 | TLL | TLX | |
| | Cbfax-1, Dinhr-1^{c}, | fax-1 | CG16801 | PNR | |
| | Srnhr-4 | | | | |
| NR2F | Cbunc-55 | unc-55 | Seven-up | COUP-TF1, | |
| | | | | COUP-TF2, | |
| | | | | EAR2 | |
| NR4A | Bpnhr-1, Cbnhr- | nhr-6 | HR38* | NGFI-Bα, β, γ | 22,24 |
| | 6, Dinhr-2^{c}, | | | | |
| | Minhr-8 | | | | |
| NR5A | Bmnhr-9, | nhr-25 | FTZ-F1* | SF1, LRH1 | 24 |
| | CbCB015N01, | | | | |
| | Srnhr-3, Ssnhr-3 | | | | |
| NR6? | Cbnhr-91, | nhr-91 | HR4 | GCNF? | 24 |
| | Ssnhr-4 | | | | |
| ^{a}The relationships among NR sequences were assessed as for Table 1. | | | | | |
| ^{b}C. briggsae (Cb) sequences are indicated by the name of the corresponding genomic clone for completed fosmid sequences (see the Web site having URL genome.wustl.edu/gsc/Projects/briggsae.shtml) or named based on the C. elegans ortholog. For previously unnamed genes from parasitic nematodes, the inventors have assigned gene names in accordance with established conventions^{67,68} - the first two | | | | | |
| #letters indicate species of origin, and the remainder of each gene name follows the C. elegans nomenclature conventions. The species represented are Ascaris suum (As), Brugia malayi (Bm), Brugia pahangi (Bp), Dirofilaria immitis (Di), Globodera rostienchus (Gr), Meloidogyne incognita (Mi), Onchocerca volvulus (Ov), Strongyloides ratti (Sr), Strongyloides stercoralis (Ss), and Trichinella spiralis (Ts). | | | | | |

^{c}Indicated sequences are from K. Crossgrove, G. Tzertzinis, C. Shea, and C. Maina (in preparation). All other sequences are present in public databases (as of March 2002).
^{d}The Drosophila NRs involved in the ecdysone-response cascade¹³ indicated with*.

TABLE 3
Nuclear receptors from nematodes other than C. elegans: Numbers of
divergent NR sequences^{a}

| | | | Insufficient |
|---|---|---|---|
| | NRs with apparent | NRs without | sequence |
| | C. elegans | C. elegans | to assess |
| Nematode species | orthologs | orthologs | orthology |
| Brugia malayi | 1 | 1 | |
| Caenorhabditis | 10 | 7 | 3 |
| briggsae | | | |
| Haemonchus contortus | 1 | | |
| Meloidogyne incognita | 1 | 5 | 3 |
| Pristionchus pacificus | 1 | | 2 |
| Strongyloides | 1 | | |
| stercoralis | | | |
| Trichinella spiralis | | | 1 |
| Zeldia punctata | | | 1 |

^{a}Based on sequences available in GenBank as of December 2000 (see the Web site having URL www.ncbi.nlm.nih.gov/). The relationships among NR sequences were assessed as for Table 1. NR sequences that were not placed stably into one of the six currently defined NR subfamilies are tabulated here as divergent NRs. At present, only one divergent nematode NR gene has been assigned a formal designation (C. elegans odr-7, NR0A4).

TABLE 3a
Nuclear receptors from nematodes other than C. elegans: Numbers of
divergent NR sequences^{a}

| | | | Insufficient |
|---|---|---|---|
| | NRs with apparent | NRs without | sequence |
| | C. elegans | C. elegans | to assess |
| Nematode species | orthologs | orthologs | orthology |
| Ancylostoma caninum | 1 | 1 | |
| Ascaris suum | 1 | 2 | 2 |
| Brugia malayi | 3 | 2 | |
| Caenorhabditis | 104 | 118 | |
| briggsae | | | |
| Haemonchus contortus | 1 | | |
| Heterodera glycines | | 1 | |
| Meloidogyne incognita | 2 | 5 | 5 |
| Meloidogyne javanica | | 1 | 1 |
| Parastrongyloides | 2 | 1 | |
| trichosuri | | | |
| Pristionchus pacificus | 3 | | 2 |
| Strongyloides ratti | 1 | 1 | |
| Strongyloides | 2 | | 3 |
| stercoralis | | | |
| Trichinella spiralis | | | 1 |
| Zeldia punctata | | | 1 |
| ^{a}Based on sequences available in GenBank as of March 2002 (see the Web site having URL www.ncbi.nlm.nih.gov/) and, for C. briggsae, genomic sequence available from the Washington University Genome Sequencing Center (see the Web site have URL http://genome.wustl.edu/projects/cbriggsae/). The relationships among NR sequences were assessed as for Table 1. NR sequences that were not placed stably into one of the | | | |
| #six currently defined NR subfamilies are tabulated here as divergent NRs. At present, only one divergent nematode NR gene has been assigned a formal designation (C. elegans odr-7, NR0A4). | | | |

As the nematode most used in laboratory studies, C. elegans has provided the majority of our current insights into the biological functions of nematode NRs. Five of the C. elegans NR genes are known to correspond to loci first identified in phenotypic screens. Three of these genes regulate aspects of neural differentiation: odr-7, which encodes a highly divergent NR, is required for full function of a pair of specific chemosensory neurons²⁵; unc-55 controls post-embryonic remodeling of the synaptic specificity of particular motor neurons²⁶; and fax-1 regulates axon pathfinding and neurotransmitter expression in specific interneurons, and might also contribute to the differentiation of other neurons²⁷. The UNC-55 and FAX-1 proteins are members of the conserved COUP and PNR NR groups, respectively. Notably, vertebrate and insect members of these families have also been implicated in regulation of neural development²⁸⁻³⁰, suggesting that neural specification could be an ancient function of these particular NRs.

The two other C. elegans NRs definitively correlated with mutationally defined loci (both members of conserved NR subfamilies, Table 1) function in multiple cell types to regulate more global aspects of nematode development. SEX-1 is a key component of the X-chromosome-counting mechanism that acts during early embryogenesis to control the activity of the central sex-determination and dosage compensation switch gene xol-1, thereby linking sexual fate to X-chromosome number³¹. DAF-12 is required for development of the dauer larva, an alternative, diapaused third larval stage of variable duration through which C. elegans develops when environmental conditions do not favor continuous growth³². DAF-12 also regulates developmental age, integrating signals reflecting environmental conditions with chronological stage to specify appropriate cellular programs during larval development³³. The dauer stage shares many physiological characteristics with the infective stages of parasitic nematodes³⁴. The identification of apparent daf-12 orthologs from the animal parasites Strongyloides stercoralis^{32,35} and Dirofilaria immitis (Claude Maina, unpublished personal communication) suggests that this component of the genetic pathway regulating dauer development is conserved among nematodes.

Reverse genetic analysis (e.g., the isolation of mutants bearing deletions in targeted genes³⁶ and the specific disruption of gene function by double-stranded-RNA-mediated interference (RNAi)³⁷) has revealed functions for the genes nhr-23, nhr-25, and nhr-8, each of which is a member of a conserved NR subfamily. nhr-8 is discussed extensively below. Both nhr-23 and nhr-25 are required for proper epidermal morphogenesis during embryogenesis³⁸⁻⁴⁰; nhr-25 is also required for development of the somatic gonad^{39,40} In addition, interference with the function of either gene causes defects in cuticle shedding during the larval molts³⁸⁻⁴⁰. Post-embryonic RNAi is sufficient to induce the cuticle shedding defects (Kostrouchova M, Krause M, Kostrouch Z, Rall J E (2001): Nuclear hormone receptor CHR3 is a critical regulator of all four larval molts of the nematode Caenorhabditis elegans. Proc Natl Acad Sci U S A 98:7360-5.; Gissendanner C R (2001): Functional Analysis of Nuclear Receptor Genes in the Nematode Caenorhabditis elegans: Ph.D. Dissertation. Athens, Ga.: University of Georgia), suggesting that these genes have direct roles in execution of the molt. This observation provides an intriguing parallel to the insect NRs HR3 and FTZ-F 1 (orthologs of NHR-23 and NHR-25, respectively), which are components of the hormone-response pathway controlling insect molting and metamorphosis¹³. Apparent functional redundancy among eight closely related NR genes, all of which are expressed in the same subset of epidermal cells, thwarted efforts to determine loss-of-function phenotypes, although overexpression experiments provided evidence for tissue-specific function¹⁶. nhr-2 is one of the earliest genes to be transcribed during embryogenesis, and although the results of functional studies are consistent with a role for nhr-2 in embryogenesis, the precise role has yet to be determined⁴¹ (T. H. Lindblom, Ph.D. dissertation, University of Georgia, 2000).

Even though the functions have thus far been probed for only a small subset of C. elegans NR genes, the results have already revealed a broad range of biological roles. The functions of several nematode members of conserved NR families are reminiscent of those of their counterparts in insects or vertebrates, as might be expected for genes representing ancient characteristics of metazoans. The corollary expectation is the more divergent nematode NRs will function in more derived aspects of nematode biology.

All of the nematode NRs are orphan receptors, i.e., no ligand (either endogenous or endogenous) has been identified for these receptors. Although no nematode steroid or terpenoid hormones have yet been described, nematodes have been reported to produce steroids not detected in other phyla (43,44) and such molecules could serve as NR ligands. In addition, there is evidence indicating that aspects of nematode development are regulated by broadly diffusible signals⁴⁴⁻⁴⁷. Furthermore, immunoassays have detected ecdysteroids in the larvae of several parasitic nematodes^{50,51}. The effects of exogenous ecdysteroids on several parasitic nematodes provides evidence for nematode responsiveness to such hormones⁵²⁻⁵⁷. Thus considerable data suggests conservation of function between nematode NRs and those found in other species.

The evidence most suggestive of steroid hormone modulation of nematode development has come from studies of ecdysteroids and nematode parasites of mammals, providing one candidate ligand for a nematode NR. Ecdysteroids were first described in insects, where the biologically active form, 20-hydroxyecdysone, is a key hormonal regulator of molting and metamorphosis. The insect ecdysone receptor is a heterodimer of two NRs, EcR and Usp. Upon binding of hormone, the receptor activates a cascade of transcriptional regulators that includes several orphan NRs¹³. Immunoassays have detected ecdysteroids in the larvae of several parasitic nematodes, including Dirofilaria immitis, Ascaris suum, Onchocerca volvulus, and Onchocerca gibsoni^{50,51}, although only for O. gibsoni were accompanying mammalian host tissue and blood samples also directly tested and ruled out as a source of potential cross-reacting material.

The effects of exogenous ecdysteroids on several parasitic nematodes provide evidence for nematode responsiveness to such hormones. For the intestinal parasites Nematospiroides dubius⁵² and Ascaris suum⁵³, larval molting can be stimulated in vitro by ecdysteroid concentrations in the nanomolar range, consistent with the effective concentration in insects. Similarly, molting of third stage larvae of the filarial parasite D. immitis can be stimulated by micromolar concentrations of 20-hydroxyecdysone or the non-steroidal ecdysone agonist RH5849^{54,55}. Micromolar concentrations of ecdysone also induce reinitiation of oocyte meiosis in D. immitis ovaries and release of progeny by Brugia pahangi females⁵⁶; the latter process is also inhibited by proposed disruptors of steroid hormone metabolism⁵⁷. These results suggest a potential role for ecdysteroids in the regulation of molting and other developmental processes in these nematodes. Consistent with this hypothesis, EcR and Usp orthologs have been identified in D. immitis and B. malayi. Recent studies of invertebrate molecular phylogeny have suggested that nematodes and arthropods reside in a common evolutionary clade, the “Ecdysozoa”⁵⁸⁻⁶⁰, for which molting is proposed to be a defining shared trait⁵⁸. One consequent expectation is that key elements of the molting regulatory circuitry should be conserved among Ecdysozoa members, and ecdysteroid regulation of molting through EcR and Usp orthologs in nematodes would be appealingly consistent with this hypothesis. However, observations from C. elegans indicate that any conservation of molting regulation is more complex than simple universal preservation of the ecdysone hormone and receptor. C. elegans does not produce ecdysteroids^{54,61} and, as noted above, lacks evident orthologs of EcR and Usp¹⁵. Furthermore, no effects of ecdysteroids on C. elegans have been reported. However, C. elegans does have apparent orthologs of orphan NRs in the insect ecdysone response cascade (Table 1), and at least two of these are required for proper completion of larval molts (see discussion above of NHR-23 and NHR-25). Thus the genetic pathway regulating execution of the molt might be generally conserved among “Ecdysozoans”, whereas the primary signal for initiating a molt is likely to differ from arthropod ecdysteroids in at least some nematodes, including C. elegans. The nature of this signal in C. elegans, hormonal or otherwise, remains to be determined, as does the extent to which it could function in other nematodes or phyla.

Among the known functions of other nematode NRs, that of DAF- 12 in progression of the C. elegans life cycle is particularly suggestive of a role for a yet-to-be-identified hormonal ligand. DAF-12 regulates the coordinate selection of life-stage-specific cellular programs in diverse cell types³³. The inappropriate reiteration of characteristics of earlier stages that occurs in some daf-12 mutants is reminiscent of that observed in lepidopterans exposed to exogenous juvenile hormone⁶². Furthermore, several daf-12 missense mutations identified in phenotypic screens alter predicted ligand contact sites³², consistent with ligand regulation of DAF-12 protein activity.

Although ligand regulation would not be inconsistent with the biological roles of the other nematode NRs with known functions, it is also not required to explain existing observations. The extent to which the large diversity of nematode NRs might be matched by a diversity of ligands remains to be learned. Organisms with the relatively small size and rapid development exhibited by many nematodes seem unlikely candidates for employing a sufficiently large number of hormones in the classic sense to utilize 270 nuclear receptors, and many of the nematode NRs might function independently of ligands. However, one potential source of a diversity of ligands is the external environment, as proposed herein for NHR-8. While not wishing to be bound by any theory, the inventors propose that NRs that respond to exogenous compounds and regulate expression of appropriate metabolic or developmental pathways could be one mechanism by which nematodes adapt to exploit or to survive their environment. For parasitic nematodes, such adaptation could include responding to the hormonal state of the host or vector organism. For some vertebrate NRs, cytoplasmic functions distinct from their nuclear, gene-regulatory roles have been described⁶⁴⁻⁶⁶, and an expansion of NR functions other than transcriptional regulation could have been one factor contributing to the diversification of nematode NRs.

Crystallographic studies have been reported for six NR LBDs (48-55), revealing a common tertiary structure comprising 11 or 12 alpha-helices surrounding a core ligand-binding “pocket”. Conservation of key primary sequence elements suggests that the LBDs of many orphan receptors share this tertiary structure (18, 56). The inventors have recognized that analysis of the C. elegans LBD sequences by structural modeling indicates that the majority of the C. elegans LBDs are likely to exhibit the 12 helix tertiary structure defined by crystallographic studies. Thus the majority of the C. elegans NRs and likely NRs found in other nematode species exhibit the structural potential for modulation of their activity by the binding of specific chemical compounds.

G. Nematode NRs as Targets for Anti-Nematode Agents

The present invention encompasses the recognition that nematode NRs represent attractive targets for the development of anti-nematode agents, e.g., pharmaceuticals or pesticides. Identification of a molecule that can alter the function of a given NR can provide a basis for such development efforts even if the natural ligand is unknown. For example, identification of 9-cis retinoic acid as a ligand for the retinoid X receptors (RXRs) (45,46) provided a springboard for the development of synthetic retinoid ligands for RXR (e.g., 27,47), notwithstanding the fact that it remains unclear whether 9-cis retinoic acid is a natural ligand for RXR (see discussion in reference 20). Similarly, identification and/or development of anti-nematode agents does not require a priori the identification of an endogenous nematode NR ligand, but needs only the identification of a molecule that can bind to and affect the activity of a nuclear receptor required for nematode growth or viability.

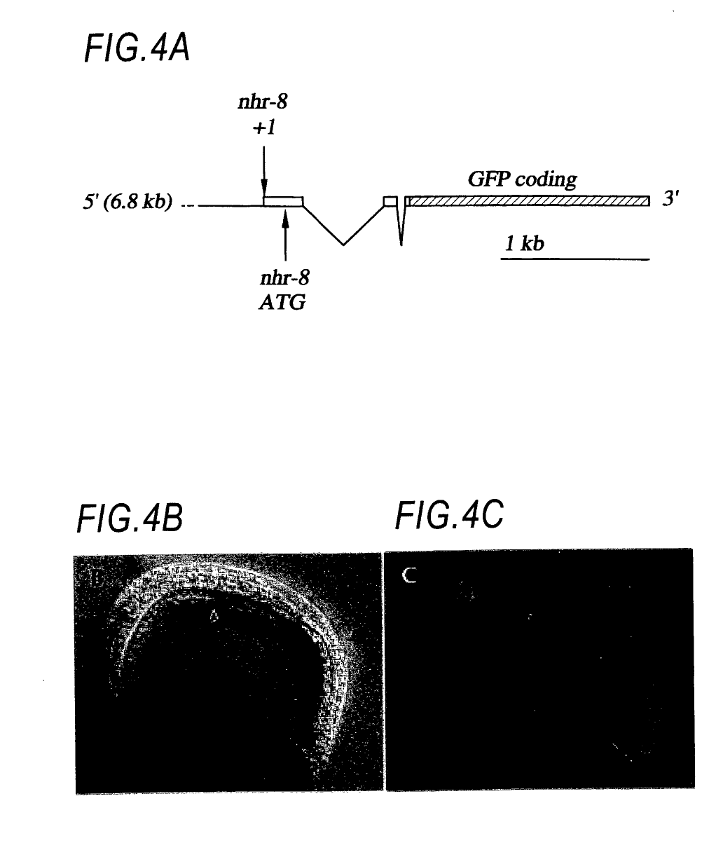

While a variety of nematode NRs are attractive candidates for discovery of anti-nematode agents, the present invention focuses on xenobiotic nematode NRs. As further described in Example 1, the inventors have discovered that certain nematode NRs display significant homology to vertebrate xenobiotic sensing NRs. Based on phylogenetic analysis inventors have determined that these nematode NRs fall into the NR1I/J group as do known vertebrate xenobiotic sensing NRs. (see Example 1). Furthermore, the expression pattern of certain of these nematode NRs suggests that they are likely to be involved in metabolic pathways. The inventors have discovered (see Example 2 and FIG. 4), that the C. elegans nhr-8 promoter is active in the nematode gut. In vertebrates, NR activated metabolism of xenobiotic compounds occurs primarily in the liver. Nematodes do not have a liver, and many of the functions of vertebrate hepatic tissues are performed by the nematode gut. That PXR, CAR, etc., act as xenobiotic sensors in tissues analogous to the nematode gut suggests conservation of function among members of this NR subfamily. In addition, numerous CYP-450 sequences are present in nematode genomes. For example more than 60 CYP sequences have been identified in the C. elegans genome (Gotoh, O., Divergent structures of Caenorhabditis elegans cytochrome P450 genes suggest the frequent loss and gain of introns during the evolution of nematodes. Mol. Biol. and Evolution, 15: 1447-1459, 1998.) Thus nematode members of the xenobiotic sensing NR subfamily potentially regulate metabolism of xenobiotics through effects on transcription of CYPs as is the case in vertebrates. Inventors have shown (see Example 7) that a mutation in the gene encoding the C. elegans NR NHR-8 increases the sensitivity of C. elegans to toxic xenobiotics that are known to induce CYP450 expression. While not wishing to be bound by any theory, this result suggests that when wild type nematodes are exposed to such xenobiotics, the NHR-8 protein causes increased expression of certain CYP450s, which then act to detoxify, e.g., to metabolize, the xenobiotics. A reduction in NHR-8 function may thus impair the induction of certain CYP450s, reducing the ability of the nematodes to detoxify xenobiotics and resulting in increased sensitivity.

In addition to CYPs, both vertebrates and nematodes contain other proteins that function to reduce the toxicity of xenobiotics. Some of these proteins likely remove toxins via direct export. For example, simultaneous loss of two endodermal ABC transporter proteins, MRP- 1 and PGP-1, renders C. elegans more sensitive to heavy metals such as cadmium and arsenite [19], while loss of a third transporter, PGP-3, increases sensitivity to the plant-derived toxins colchicine and chloroquine [20]. Similarly, it is well known that transporter proteins such as P-glycoprotein pumps mediate resistance of cancer cells to certain chemotherapeutic agents. The present invention encompasses the realization that xenobiotic sensing NRs may function at least in part by altering transcription of genes encoding proteins that play a role in biotransformation, metabolism, detoxification, elimination, etc., of xenobiotics. Such genes include, but are not limited to, genes encoding CYP450s, transporters, hydrolyases, sulfatases, glutathione S-transferases and UDP-glucuronyltransferases (See, e.g., Sheweita S A. 2000. Drug metabolizing enzymes: Mechanisms and functions. Current Drug Metabolism 1:107-132; Radominska-Pandya A, Czernik P J, Little J M, Battaglia E, Mackenzie P I. 1999. Structural and functional studies of UDP-glucoronosyltransferases. Drug Metabolism Reviews 31: 817-899; Sheehan D, Meade G, Foley V M, Dowd C A. 2001. Structure, function and evolution of glutathione transferases: implications for classification of non-mammalian members of an ancient enzyme superfamily. Biochemcial Journal 360 (Pt. 1): 1-16). For the sake of brevity, such genes will be referred to herein as genes encoding a xenobiotic metabolizing protein.

The term “nematode xenobiotic sensing NR” as used herein includes any nematode NR that is a homolog, ortholog, or paralog of a vertebrate xenobiotic sensing NR (e.g., SXR/PXR, PAR, CAR, CXR). The term “vertebrate xenobiotic sensing NR” includes any vertebrate NR that has been shown to respond to any xenobiotic by either (1) causing an alteration in the sensitivity of the vertebrate to any xenobiotic (which may or may not be a xenobiotic to which the vertebrate NR responds); (2) causing an alteration in the metabolism, biotransformation, or distribution of any xenobiotic (which may or may not be a xenobiotic to which the vertebrate NR responds); (3) causing an alteration in the expression of a vertebrate CYP gene or other gene that encodes a protein that plays a role in xenobiotic metabolism, biotransformation, distribution, elimination, etc.; or (4) binding with high affinity to one or several xenobiotics or with low affinity to several or more different xenobiotics. Whether a particular NR is considered a xenobiotic sensing NR can be determined using any of a variety of available techniques that are well known in the art. Such techniques include, for example, showing direct ligand modulation of the NR, or demonstrating that expression of the NR in a setting other than that in which it is naturally found (such as a transfected cell line derived from either the same or a different species, a transgenic animal) causes alteration in sensitivity to a xenobiotic (which may or may not be a xenobiotic to which the vertebrate NR responds), alteration in metabolism, biotransformation, distribution, or elimination, of a xenobiotic (which may or may not be a xenobiotic to which the vertebrate NR responds), or alteration in the expression of a vertebrate CYP gene or other gene that encodes a protein that plays a role in xenobiotic metabolism, biotransformation, distribution, elimination, etc. The likelihood that an NR is a xenobiotic sensing NR can also be demonstrated genetically, e.g., by showing change in sensitivity to a xenobiotic (which may or may not be a xenobiotic to which the vertebrate NR responds), alteration in metabolism, biotransformation, distribution, or elimination of a xenobiotic (which may or may not be a xenobiotic to which the vertebrate NR responds), or alteration in the expression of a vertebrate CYP gene in a mutant such as a knockout mutant (or any other mutant in which the expression level of the NR is reduced and/or part or all of the NR is deleted or altered).

The term “nematode xenobiotic sensing NR” includes C. elegans NHR-8 and homologs, paralogs or orthologs thereof found in other nematode species. In certain embodiments of the invention the nematode species is a parasitic nematode species, which can be a member of any of the following nematode orders: Strongylida, Rhabditida, Ascaridida, Spirurida, Oxyurida, Enoplida, Tylenchida, and Dorylaimida. In certain embodiments of the invention the parasitic nematode is an animal parasite and is a member of a genus selected from the list of genera presented in Table 4 under the orders Strongylida, Rhabditida, Ascaridida, Spirurida, Oxyurida, or Enoplida. In certain embodiments of the invention the parasitic nematode is a plant parasite and is a member of a genus selected from the list of genera presented in Table 4 under the orders Tynlenchida or Dorylaimida. Other parasitic nematodes are also within the scope of the invention. References describing the phylogeny and classification of nematodes include, for animal parasitic nematodes: R C Anderson, Nematode Parasites of Vertebrates, 2nd ed., CAB International, 1992, and for plant-parasitic nematodes: V H Dropkin, Introduction to Plant Nematology, 2nd ed., Wiley, 1989.

TABLE 4
Parasitic Nematode Orders and Genera

| | | Common name or | Phylog netic |
|---|---|---|---|
| Order | Host | disease name | family |
| Strongylida | | | |
| Haemonchus | ungulates | | |
| Oestertagia | sheep, cattle, goats | | |
| Trichostrongylus | cattle | | |
| Cooperia | cattle | | |
| Dictyocaulus | horses, ruminants | | |
| Strongylus | horse | | |
| Oesophagostomum | pigs, ruminants | | |
| Syngamus | poultry | | |
| Nematodirus | sheep, goats | | |
| Heligmosomoides | rodents | | |
| Nippostrongylus | rodents | | |
| Metastrongylus | pig | | |
| Angiostrongylus | humans | | |
| Acyclostoma | human | hookworm | |
| Necator | human | hookworm | |
| Uncinaria | dogs etc | dog hookworm | |
| Bunostomum | cattle hookworm | | |
| Rhabditida | | | |
| Strongyloides | human | | |
| Steinernema | insects | | |
| Ascaridida | | | |
| Ascaris | human | roundworm | |
| Parascaris | horses | | |
| Toxocara | dogs | | |
| Toxascaris | cats, dogs | | |
| Baylisascaris | raccoons | | |
| Anisakis | fish - human | | |
| Pseudoterranova | fish - human | | |
| Heterakis | poultry | | |
| Spirurida | | | |
| Wuchereria | human | elephantiasis | |
| Brugia | human | | |
| Onchocerca | human | river blindness | |
| Dirofilaria | dog | | |
| Loa | human | | |
| Thelazia | mammal, bird eyelid | | |
| Dracunculus | human | guinea worm | |
| Gnathostoma | human | | |
| Oxyurida | | | |
| Enterobius | human | | |
| Oxyuris | horses | | |
| Syphacia | human | | |
| Enoplida | | | |
| Trichinella | human | | |
| Trichuris | human | | |
| Capillaria | human | | |
| Tylenchida | | | |
| Globodera | plants | (potato) cyst nematode | Heteroderidae |
| Heterodera | plants | (soybean) cyst nematode | Heteroderidae |
| Meloidogyne | plants | root knot nematode | Heteroderidae |
| Anguina | plants, wheat | seed, stem & leafgall nematode | Tylenchidae |
| Ditylenchus | plants | potato rot nematode | Tylenchidae |
| Hirschmanniella | plants | rice root nematode | Pratylenchidae |
| Naccobus | plants | false rootknot nematode | Pratylenchidae |
| Pratylenchus | plants | lesion nematode | Pratylenchidae |
| Radopholus | plants | burrowing nematode | Pratylenchidae |
| Criconema | plants | ring nematode | Criconematidae |
| Tylenchulus | plants | citrus nematode | Tylenchulidae |
| Paratylenchus | plants | pin nematode | Paratylenchidae |
| Aphelenchus | plants | bud & leaf nematode | Aphelenchidae |
| Bursaphelenchus | plants | pinewood nematode | Aphelenchoididae |
| Dorylaimida | | | |
| Longidorus | plants | needle nematode | Longidoridae |
| Xiphinema | plants | dagger nematode | Longidoridae |
| Trichodorus | plants | stubby root nematode | Trichodoridae |
| Paratrichodorus | plants | stubby root nematode | Trichodoridae |

As discussed below, the inventors have shown that C. elegans in which the gene encoding NHR-8 is partially deleted or in which expression of NHR-8 is eliminated or greatly reduced show increased sensitivity to the xenobiotics colchicine and chloroquine, consistent with a role for NHR-8 in xenobiotic sensing (see Examples 4 and 5). Furthermore, as shown in Example 7, inventors have demonstrated that C. elegans in which the gene encoding NHR-8 is partially deleted or in which expression of NHR-8 is eliminated or greatly reduced show increased sensitivity to the xenobiotics primaquine and atrazine, which have long been known to induce expression of CYP450 genes in other organisms. The target genes regulated by NHR-8 may include CYP450 genes as well as genes encoding other xenobiotic metabolizing enzymes including, but not limited to, those mentioned above. These results suggest that NHR-8 responds to certain xenobiotics in a manner analogous to that described above for the vertebrate xenobiotic sensing NRs and confirm the utility of NHR-8 as a target for anti-nematode compounds.

The inventors have also recognized that the C. briggsae gene known as CbG02P14 is an ortholog of the nhr-8 gene. Furthermore, the inventors have discovered a probable ortholog of nhr-8 in the parasitic nematode Haemonchus contortus. These results suggest that existence of xenobiotic sensing NRs is a general characteristic of nematodes. The term “nematode xenobiotic sensing NR” includes these identified orthologs and also includes C. elegans NHR-48, which is closely related in sequence to NHR-8, as well as homologs, orthologs and paralogs of NHR-48 in other nematode species. The term also includes C. elegans DAF-12, which is closely related in sequence to NHR-8, as well as homologs, orthologs and paralogs of DAF-12 in other nematode species. In certain embodiments of the invention the term further includes any nematode NR that is a paralog or ortholog of a vertebrate xenobiotic sensing NR. In certain embodiments of the invention the term additionally includes any nematode member of the NR1I/J subfamily, wherein membership in the subfamily may be ascertained, for example, as described in Example 1. The term additionally includes any nematode NR that has been shown to respond to any xenobiotic by either (1) causing an alteration in the sensitivity of the nematode to any xenobiotic (which may or may not be a xenobiotic to which the nematode NR responds); (2) causing an alteration in the metabolism, biotransformation, or distribution of any xenobiotic (which may or may not be a xenobiotic to which the nematode NR responds); (3) causing an alteration in the expression of a nematode CYP450 gene other gene that encodes a protein that plays a role in xenobiotic metabolism, biotransformation, distribution, elimination, etc.; (4) binding with high affinity to one or several xenobiotics or with low affinity to several or more different xenobiotics. Methods for determining whether a nematode NR is a homolog, paralog or ortholog of a vertebrate xenobiotic sensing NR are discussed herein and are known to one of ordinary skill in the art of molecular comparison. In addition, as discussed above, methods for determining whether an NR (either a vertebrate or nematode NR) responds to a xenobiotic in any of the four ways mentioned above are known to one of ordinary skill in the art and are described in the scientific literature referred to herein.

In certain embodiments of the invention where the NR is a parasitic nematode NR, the protein exhibits a significant degree of similarity (homology) to a known member of the NR1I/J subfamily such as C. elegans nhr-8, nhr-48, or daf-12 or a vertebrate or insect member of the subfamily. The degree of similarity can be determined using a variety of approaches and can take into consideration both the degree of identity between two sequences (preferably allowing for gaps) and the existence of conservative substitutions, i.e., positions at which two amino acid sequences differ but where the two amino acids exhibit similar biochemical characteristics with respect to size, charge, polarity, etc. Examples of conservative substitutions are well known in the art. See, for example, Biochemistry, 4th Ed., Stryer, L., et al., W. Freeman and Co., 1995 and U.S. Pat. No. 6,015,692. In certain preferred embodiments of the invention, when aligned so as to produce maximum similarity while allowing for gaps, at least 25% of the amino acid residues in the shorter of the two proteins are similar to the corresponding (aligned) residue in the other protein, where a first amino acid residue is similar to a second amino acid residue if it is either identical to the second residue or if a substitution of the second amino acid for the first (or vice versa) is a conservative substitution. In certain preferred embodiments of the invention the foregoing criteria are met with respect to at least 30% of the amino acid residues. In certain preferred embodiments of the invention the foregoing criteria are met with respect to at least 40%, at least 50%, at least 60%, or at least 70% of the amino acid residues. As is well known in the art, significant homologies may extend over only a portion of the homologous proteins rather than over the full length of either protein. Thus in certain embodiments of the invention the parasitic nematode NR exhibits a significant degree of similarity to a particular NR1I/J subfamily member over one or more portions of the sequence. For example, in certain embodiments of the invention, when aligned so as to produce maximum similarity while allowing for gaps, at least 50% of the amino acid residues in the parasitic nematode NR and the NR1I/J subfamily member are similar to the corresponding (aligned) residue in the other protein over a domain at least 20 amino acids in length. In other embodiments the foregoing criteria are met with respect to at least 60%, at least 70%, at least 80%, or at least 90% of the amino acids over a domain at least 20 amino acids in length. In yet other embodiments of the invention the length of the domain over which the NRs exhibit such similarity is at least 30 amino acids, at least 50 amino acids, at least 75 amino acids, at least 100, at least 150, or at least 200 amino acids in length. The foregoing discussion providing various methods of determining whether two proteins display significant homology is intended to be illustrative and should not be considered to be limiting. In general, one of ordinary skill in the art will be able to determine whether two proteins display sufficient similarity to be considered homologs of one another.

Various computer programs mentioned above provide an indication of the similarity between homologous sequences. For example, the BLAST programs mentioned above provide a score that reflects the degree of homology and also provide value referred to as an E value for each sequence identified as homologous to the input sequence. The E value represents the number of alignments with an equivalent or greater score that would be expected to occur purely by chance, and therefore alignments with a low E value are likely to be significant. Thus in certain embodiments of the invention the alignment between a parasitic nematode NR and a homologous NR1I/J subfamily member has an E value of less than 0.001, and yet more preferably less than 0.0001. In certain embodiments of the invention a homologous parasitic nematode NR has a score of at least 80 when compared with a corresponding NR1I/J subfamily member using the BLASTP program (assuming default parameters).

The inventors have recognized that modulating or altering the activity or amount of a nematode xenobiotic sensing NR has the potential to increase the effectiveness of one or more known (or as yet undiscovered) anti-nematode agents whose metabolism may be regulated or influenced by such NRs. In the case of a xenobiotic sensing NR that responds to a xenobiotic by causing (either directly or indirectly) an increase in the metabolism, biotransformation, and/or detoxification of an anti-nematode agent, antagonizing the activity and/or reducing the amount of the xenobiotic sensing NR would allow a smaller quantity of the anti-nematode agent to be employed. Inclusion of one or more antagonist(s) of a xenobiotic nematode NR in a mixture with one or more anti-nematode agents would thus be of significant utility. An antagonist of a xenobiotic sensing nematode NR may act synergistically with another anti-nematode agent in that the total anti-nematode activity of a combination of an antagonist of a xenobiotic sensing nematode NR and a second anti-nematode agent may be greater than the sum of the effects of each agent taken individually. A reduction in the required dose of the second anti-nematode agent would reduce side effects (in the case of a anti-nematode pharmaceutical) and reduce deleterious effects on non-pest organisms (in the case of an anti-nematode pesticide). Reduction of required dose could also yield a significant savings in cost of production of effective treatment, if the antagonist is cheap and the second agent is expensive to produce (e.g., if the first is a small molecule that can be synthesized in bulk, and the second is a natural product that has to be obtained by fermentation). In addition, inhibiting metabolism with an antagonist of a xenobiotic sensing nematode NR may expand the arsenal of compounds available to combat nematodes by allowing the use of compounds which would normally be metabolized too rapidly to be effective, or in which sufficiently high concentrations cannot be achieved in a practical manner due to metabolism, biotransformation, etc.

Precedents for synergism between a first compound and a second compound whose activity reduces metabolism of the first compound are known. For example, 1) In insects piperonyl butoxide and MB-599 act synergistically; 2) In bacteria clavulanic acid (and a related compound, sulbactam), inhibit the bacterial enzyme beta lactamase that metabolizes penicillins and cephalosporins that would normally be degraded by plasmid or chromosomal beta lactamases in resistant bacteria, thus these compounds synergize with penicillins and cephalosporins. These inhibitors resuscitated the beta lactam usage and markets, in the form of amoxicillin-clavulanate (Augmentin), sulbactam-ampicillin (Unasyn), and ticarcillin-clavulanate (Timentin).

Although most NRs studied to date act as transcriptional activators whose activity is increased in the presence of ligand, some NRs act as transcriptional repressors, either in the absence or presence of ligand. Although vertebrate xenobiotic sensing NRs studied to date appear to act as transcriptional activators of CYP genes, it is possible that some xenobiotic sensing NRs instead act as transcriptional repressors, e.g., they may constitutively repress CYP genes in the absence of ligand, allowing binding of ligand to release repression and lead to an increase in CYP transcription. Thus in certain embodiments of the invention an agonist of a nematode xenobiotic sensing NR, e.g., a compound that decreases the activity and/or amount of an NR that represses transcription may be employed. In the most general sense, whether it is more appropriate to employ an antagonist or agonist of a nematode xenobiotic sensing NR depends upon the ultimate effect of modulating the activity of the nematode xenobiotic sensing NR on metabolism, biotransformation, and/or detoxification of a xenobiotic of interest such as an anti-nematode agent. One of ordinary skill in the art will be able to determine, for any particular nematode xenobiotic sensing NR, whether an antagonist or agonist is more appropriate. For example, as described herein, since reduction in activity of the nematode xenobiotic sensing NR NHR-8 results in an increase in sensitivity to toxins, an antagonist would be suitable to increase toxin efficacy.

It is also noted that although vertebrate xenobiotic sensing NRs studied to date appear to exert their effects by increasing transcription of CYP-450 genes, it is possible that nematode xenobiotic sensing NRs may instead activate (or repress) transcription of other genes involved in the metabolism, biotransformation, and/or detoxification of xenobiotics. It is noted that the methods and approaches described herein are applicable to pest species other than nematodes, e.g., insect pests. As described herein, insects have NRs, including members of the NR1I/J subfamily. For example, the Drosophila melanogaster gene that encodes the protein DHR96, (Fisk & Thummel, Isolation, regulation, and DNA-binding properties of three Drosophila nuclear hormone receptor superfamily members. PNAS 92: 10604-10608, 1995), is homologous to C. elegans nhr-8 and daf-12.

(i) Identifying Nematode Xenobiotic Sensing NRs

Nematode xenobiotic sensing NRs may be identified using any of a variety of approaches. The invention encompasses searching of databases including sequence information generated by various parasite genome and EST sequencing projects to identify open reading frames encoding putative nematode xenobiotic sensing NRs. Databases containing genome and sequence information (in addition to general databases such as Genbank, EMBL, etc.) exist for a wide variety of parasites including parasitic nematodes and may be accessed at the Web site having URL www.ebi.ac.uk/parasites/parasite-genome.html. Nematode homologs, orthologs, or paralogs of vertebrate xenobiotic sensing NRs represent candidate parasitic nematode xenobiotic sensing NRs. Similarly, a database containing sequence information for a first nematode species may be searched to identify homologs, orthologs, or paralogs of a xenobiotic sensing NR that has previously been identified in a second nematode species.

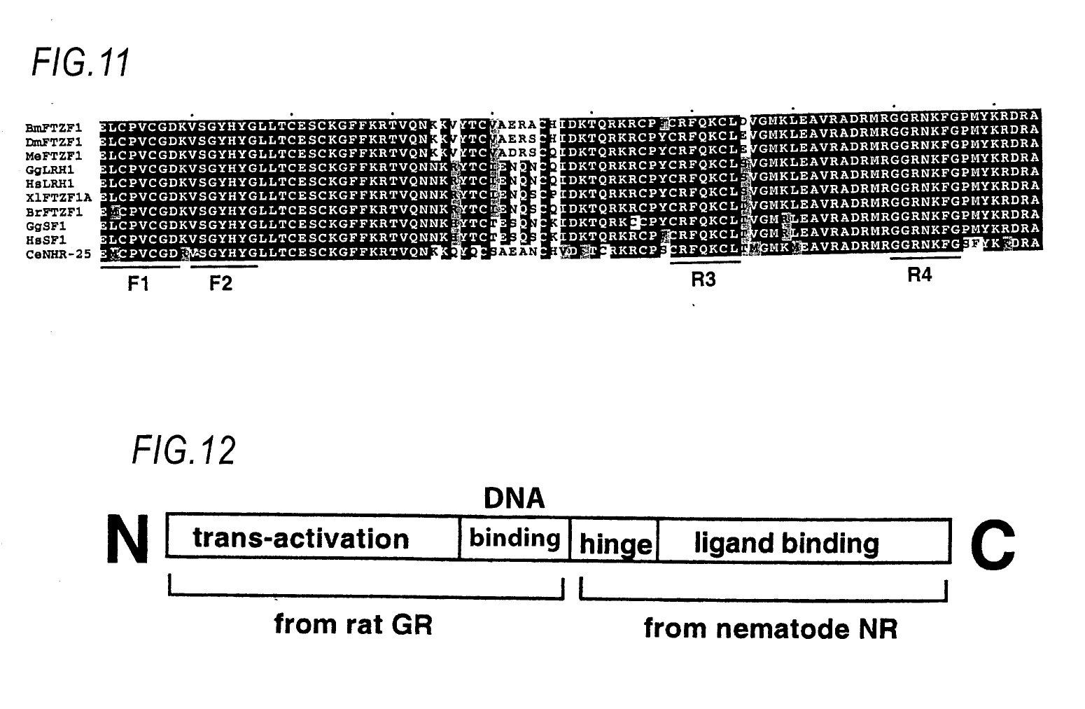

Although searching sequence databases is a convenient means of identifying parasitic nematode homologs of either vertebrate or C. elegans xenobiotic sensing NRs, other methods for identifying homologous parasitic nematode NR genes are also within the scope of the invention. For example, PCR (or RT-PCR) may be performed (typically under conditions of reduced stringency) on genomic DNA, a cDNA or genomic library (or RNA) from a parasitic nematode species of interest. PCR would be performed using a probe comprising, for example, an oligonucleotide primer, a combination of degenerate oligonucleotide primers, or a cDNA or portion thereof derived from the sequence of a known or putative vertebrate, C. elegans, or other parasitic nematode xenobiotic sensing NR. Methods for designing appropriate primers are well known in the art. In certain embodiments of the invention a PCR strategy similar to that previously used to recover C. elegans NR sequences (15) is used to identify NR sequences from the parasitic nematode species of interest. Degenerate oligonucleotide primers are designed based, e.g., on the DNA binding domain (DBD) sequences for each of the NRs of interest. A primer design strategy used for the nhr-25 gene (a C. elegans NR that is not a candidate xenobiotic sensing NR) is summarized in FIG. 11. According to the strategy a region of an NR, e.g., a C. elegans NR of interest, is aligned with homologs from other species such as insects (Bm, Dm), shrimp (Me), humans (Hs) and/or other vertebrates (Gg, X1, Br). (In FIG. 11 conserved regions selected for primer design are indicted by underlining). A mixture of nucleotides or inosine is incorporated into the degenerate (wobble) position in each codon. Primers need not be based on the DBD but may instead be based on other conserved region(s) of the NR, e.g., the ligand binding domain. One of ordinary skill in the art will readily be able to develop similar strategies for other genes encoding candidate nematode xenobiotic sensing NRs.

In certain embodiments of the invention candidate NR sequences are amplified from parasitic nematode genomic DNA using systematic variation of annealing temperatures, as described (15). Products that are amplified reproducibly are cloned and sequenced. Example 9 describes homology cloning of an homolog of the C. elegans nhr-8 gene from H. contortus genomic DNA. Presence of the cloned sequences in the genome of the species of origin can be verified, e.g., by probing a genomic Southern blot with the cloned amplification product. Although it is not necessary to use genomic DNA (e.g., mRNA or cDNA preparations may alternatively be used), genomic DNA presents a number of advantages. For example, use of genomic DNA helps to overcome potential difficulties in obtaining parasitic nematodes (especially particular developmental stages) in sufficient quantity for nucleic acid preparation. In addition, as transcription factors may exhibit limited spatial or temporal expression, NRs may not be abundantly represented in mRNA or cDNA preparations. In order to address the potential problems associated with the presence of introns, in certain embodiments of the invention multiple primers are designed for each gene class of interest; even if one primer is interrupted by an intron, the others may not be.

Although PCR represents a convenient method to initially identify a homolog of a particular NR, one could also initially screen a library produced from genomic DNA or cDNA derived from a parasitic nematode species (typically under conditions of reduced stringency) using a probe derived from a gene encoding a vertebrate, C. elegans, or other parasitic nematode xenobiotic sensing NR. As with PCR, such a probe may comprise, for example, an oligonucleotide primer, a group of degenerate oligonucleotide primers, a cDNA or portion thereof, etc.

Once a sequence encoding a candidate parasitic nematode sensing NR or a portion thereof has been identified, e.g., by PCR or by screening a library, the candidate gene may be cloned using methods well known in the art. Other techniques such as 5′ RACE may also be used to isolate further portions of the gene. (See, for example, Hawdon, et al., J. Biol. Chem., 271, 6672-6678, 1996, which describes cloning of a gene encoding the A. caninum secreted protein ASP-1.)

In addition to sequence comparisons, a number of different approaches may be employed to identify nematode xenobiotic sensing NRs and/or confirm their identity as such. In C. elegans (or other nematodes) the effect of reducing or eliminating expression or activity of a candidate xenobiotic sensing NR may be directly determined in any of a variety of ways, including assessing the growth, viability, or reproduction of the nematode in the presence of one or more xenobiotic compounds.

Another approach that is particularly powerful in organisms in which it is possible to manipulate gene expression is to reduce or eliminate expression or activity of a candidate xenobiotic sensing NR and assess the effects of so doing on the response of the organism to one or more xenobiotics. Reduction or elimination of expression or activity can be achieved either by creating a mutant (such a mutant may be, for example, a strain bearing a “knockout” of the gene that encodes the candidate NR or a strain harboring a point mutation, deletion, etc. in the gene). In certain organisms, including C. elegans, another approach known as double-stranded RNA interference (RNAi) may be employed. Double-stranded RNA (dsRNA) corresponding to a gene of interest (such as a gene that encodes a candidate xenobiotic sensing NR) is introduced into worms either by injection, by feeding them bacteria engineered to express the dsRNA, or by soaking them in a solution containing the dsRNA (though the latter method is generally less efficient). In general, RNAi results in progeny that lack expression of the corresponding gene product, allowing a determination of the loss of function phenotype. These methods are described, for example, in PCT application WO99/32619, in Fire, et al., Potent and specific genetic interference by double-stranded RNA in Caenorhabditis elegans, Nature, 391:806-11, 1998, and in Fraser, et al., Functional genomic analysis of C. elegans chromosome I by systematic RNA interference, Nature, 408:325-330, 2000.

Another method for determining the effect of reduction or elimination of expression or activity in C. elegans involves, briefly, creating a deletion mutant in which some or all of the gene is absent as described, for example, in Liu, L., et al., High-Throughput Isolation of Caenorhabditis elegans Deletion Mutants, Genome Research, 9:859-867, 1999. Of course other methods may be used to generate C. elegans strains having a mutation in a gene of interest. Such methods include, but are not limited to, transposon-mediated mutagenesis (described, for example, in Plasterk, R., “Reverse Genetics” in Methods in Cell Biology, Vol. 48, cited above).

(ii) Screens to Identify Modulators of Nematode Nuclear Receptors

The invention provides methods and screens for identifying modulators of nematode nuclear receptors such as candidate xenobiotic sensing NRs. Studies have demonstrated that NRs from vertebrates and insects are able to regulate transcription in heterologous systems, e.g., in cells of the yeast Saccharomyces cerevisiae and that this activity can be ligand regulated (60-64). Such systems, e.g., yeast, offer an economical and efficient system that can be utilized to identify compounds able to modulate NR function. Screens in yeast can readily be performed in a high throughput format. Although the description herein assumes that the heterologous system employed to identify modulators of nematode xenobiotic sensing NRs is a yeast system, it is to be understood that, based upon the teachings herein and in the scientific literature, similar systems can readily be developed by one of ordinary skill in the art using, for example, vertebrate or insect cells, provided that suitable genetic elements that function in those cells are employed.

According to one embodiment, the inventive method of identifying a modulator of a nematode nuclear receptor comprises steps of: (i) providing a cell, wherein the cell expresses a polypeptide comprising a ligand binding domain of a nematode nuclear receptor or a portion thereof and a DNA binding domain or a portion thereof, and wherein the cell contains a reporter comprising a reporter gene and a nucleic acid that comprises a binding site for the polypeptide, wherein the nucleic acid is operably linked to the reporter gene; (ii) contacting the cell with a test compound; and (iii) determining whether the amount or activity of the reporter is increased or decreased in the presence of the test compound, wherein an increase or decrease in the amount or activity of the reporter is an indication that the test compound is a modulator of a nematode nuclear receptor. In certain embodiments of the invention an increase in the amount or activity of the reporter is an indication that the test compound is an agonist of a nematode nuclear receptor. In certain other embodiments of the invention a decrease in the amount or activity of the reporter is an indication that the test compound is an antagonist of a nematode nuclear receptor. In certain embodiments of the invention the nematode nuclear receptor is a xenobiotic sensing NR. In certain embodiments of the invention the NR is C. elegans NHR-8, NHR-48, DAF- 12, or a homolog, paralog, or ortholog thereof.

In certain embodiments of the invention the reporter gene encodes a detectable marker, e.g., the E. coli lacZ gene. In certain embodiments of the invention the DNA binding domain binds to the binding site. In certain embodiments of the invention the polypeptide further comprises a transcriptional activation domain, e.g., a domain capable of activating transcription of the reporter gene. In certain embodiments of the invention the nucleic acid comprises a promoter operably linked to the reporter gene. In various embodiments of the invention the promoter is constitutive while in other embodiments the promoter is regulatable. In certain embodiments of the invention the promoter is operably linked to the binding site, so that when a transcriptional activator binds to the binding site transcription from the promoter is increased.

In certain embodiments of the invention the nematode nuclear receptor is a xenobiotic sensing nuclear receptor. The NR can be a C. elegans NR or an NR from another nematode species, e.g., a parasitic nematode species. In certain embodiments of the invention the NR is a parasitic nematode homolog of a C. elegans nuclear receptor. In certain embodiments of the invention the NR is a member of the NR1I/J group of NRs, e.g., the C. elegans nhr-8 gene, the C. elegans nhr-48 gene, the C. elegans daf-12 gene, or a homolog, ortholog, or paralog thereof found in another nematode species such as a parasitic nematode species.

According to the invention, the general strategy for assaying the function of an NR of interest employs two components: [1] an activator expressing the NR and [2] a reporter gene in which NR binding sites provide upstream activating sequence (UAS) function or enhancer function for the promoter that controls expression of (i.e., is operably linked to) the reporter gene. In certain embodiments of the invention the activator and the reporter gene sequences are contained in plasmids, although in other embodiments of the invention one or more of the sequences encoding the activator or reporter is integrated into the genome. Where yeast is used, the NR binding sites provide UAS function. Where a system other than yeast is used, the reporter gene contains NR binding sites that provide enhancer function for the promoter that controls expression of the reporter gene.

The DNA binding sites for the nematode NRs have not yet been described, and most nematode NRs have unique sequences in the region of the DNA binding domain known to confer sequence specificity and are thus likely to bind DNA sequences distinct from those described for other NRs. However, NR ligand binding domains are capable of conferring ligand regulation to the function of heterologous DNA binding and transcriptional activation domains (65-68). The invention takes advantage of this modularity of NR structure. According to the invention, chimeric genes expressing proteins in which the LBD of interest (e.g., the LBD of a nematode xenobiotic sensing NR) is fused to a DNA binding domain for which defined binding sites are known. The use of chimeric proteins allows the evaluation of the function of multiple NR LBDs without the necessity of defining the DNA binding specificity of each NR of interest or the need to develop and standardize multiple reporter constructs.

According to one embodiment of the invention, the hinge domain (which is C-terminal to the DNA binding domain and separates it from the ligand binding domain) and ligand binding domains of a nematode NR of interest, e.g, a nematode xenobiotic sensing NR are fused to the N-terminal transactivation and DNA binding domains of the rat glucocorticoid receptor (rGR) as shown in FIG. 12. Full length rGR activates transcription in yeast in a ligand-dependent manner, whereas truncated derivatives consisting of only the transactivation and DNA-binding domains act as constitutive activators (69). The rGR sequences are obtained, for example, from the plasmid pRS424.cup-GR. Use of the CUP1 promoter to drive expression of an introduced gene is described, for example, in I. G. Macreadie, O. Horaitis, A. J. Verkuylen, & K. W. Savin, Gene 104:107-111, 1991. Sequences encoding a nematode xenobiotic sensing NR of interest are obtained, e.g, from cDNAs. For NRs for which cDNA clones are not currently available, cDNA sequences encoding the LBD may be obtained, e.g., by reverse transcription-polymerase chain reaction (RT-PCR) amplification from purified RNA. Cloned amplification products can be sequenced to ensure the absence of errors introduced by the amplification process.

According to one embodiment of the invention fusion genes generated as described above are expressed in yeast under control of the constitutive glyceraldehyde-3-phosphate dehydrogenase promoter (70). Production of a fusion protein of the expected size can be verified using Western blot analysis, e.g., using commercially available antibodies against rGR (Affinity Bioreagents, Inc.). The ability of each fusion protein to activate transcription is assessed using, for example, a reporter gene. Appropriate reporter genes include, for example, a reporter with the GREs inserted in a truncated GAL1 promoter: West R W J, R R Yocum, and M Ptashne. 1984. Saccharomyces cerevisae GAL1-GAL10 divergent promoter region: location and function of the upstream activator sequence UAS_{G}. Mol. Cell. Biol. 4:2467-2478 or a reporter in which three 26 base pair glucocorticoid response elements (GREs) inserted in a truncated CYC1 promoter regulate expression of the E. coli lacZ gene (69). Initial assessments of the ability of each fusion protein to activate reporter gene expression are carried out using yeast cultures grown on agar plates using standard methods (71). A truncated, constitutively active GR derivative lacking the DBD (69) provides a positive control for activation, while full-length GR in the absence of hormone serves as a negative control.

Any GR/nematode NR fusion proteins that do not activate expression of the reporter gene are tested by a repression assay for the ability to bind DNA (71). In this assay, binding sites for the fusion protein of interest—in this case, GREs—are inserted in the GAL1 promoter between the upstream activating sequences and the transcription initiation site. Binding of a fusion protein to the GREs will interfere with expression of the reporter gene, thus providing a means of detecting DNA binding by the fusion protein even though it lacks the ability to activate gene expression (71,72). DNA binding in this assay provides evidence that the fusion protein is properly folded. However, since some NRs do not bind DNA in the absence of activating ligands, failure to bind DNA in this assay does not necessarily imply that the fusion protein has failed to fold properly.

Fusion proteins that activate reporter gene expression provide a basis for assays for compounds capable of inhibiting fusion protein function. Those that do not activate transcription of the reporter gene provide a basis for assays for compounds that activate function.

In the case that a particular fusion protein proves deleterious to yeast a copper-inducible promoter (73) is used, which allows regulation of the levels of fusion protein expression by controlling the concentration of copper in the growth medium. This may permit identification of an expression level that minimizes the toxic effect of the fusion protein on the yeast cells while still allowing development of an effective assay.

It is noted that the foregoing description of one embodiment of the invention is provided for exemplary purposes only and is not intended to be limiting. A wide variety of similar approaches are within the scope of the invention. For example, the hinge domain may be omitted from the fusion gene. The boundaries of many LBDs are not precisely defined, and it is not necessary to employ the entire LBD in certain embodiments of the invention. For example, a portion of a LBD containing at least 50%, at least 60%, at least 70%, at least 80%, at least 90%, at least 95% or substantially the entire LBD can be used. For NRs for which a ligand is known, the boundaries of the LBD can be defined by various assays, e.g., by assessing the ability of the ligand to activate or bind to a LBD containing a deletion or truncation. In the case of an NR for which a ligand is not known or for which such assays have not been performed, the LBD may be defined by performing sequence comparisons (for example, using sequence comparison techniques described herein) to identify regions of the NR that are homologous to known LBDs and are therefore expected to comprise the LBD for that NR. Similarly, the boundaries of many DBDs are not precisely defined, and in many cases the endogenous DNA binding site is unknown. It is not necessary to employ the entire DBD in certain embodiments of the invention. For example, a portion of a DBD containing at least 50%, at least 60%, at least 70%, at least 80%, at least 90%, at least 95% or substantially the entire DBD can be used. For NRs for which a DNA binding site is known, the boundaries of the DBD can be defined by various assays, e.g., by assessing the ability of a polypeptide comprising the DBD bind to the DNA binding site and/or the ability of a polypeptide comprising the DBD with a deletion or truncation to bind to the DNA binding site. In the case of an NR for which a DNA binding site is not known or for which such assays have not been performed, the DBD may be defined by performing sequence comparisons (for example, using sequence comparison techniques described herein) to identify regions of the NR that are homologous to known DBDs and are therefore expected to comprise the DBD for that NR. Preferably the DBD or portion thereof is of sufficient length to mediate binding of the polypeptide to the DNA binding site in the reporter.

Any of a number of characterized and cloned NRs other than the rGR may be employed in the practice of the invention. A variety of different promoters other than the glyceraldehyde-3-phosphate dehydrogenase promoter may be employed to drive transcription of the activator gene. Promoters other than the CYC1 and GAL1 promoters may be employed. (See, e.g., Yeast Gene Analysis, Methods in Microbiology, Vol. 26, Alistair J. P. Brown and Mick F. Tuite (Eds.), Academic Press, San Diego, 1998; Guthrie, C. and Fink, G., Guide to Yeast Genetics and Molecular Biology, Methods in Enzymology (Vol 194), Academic Press, 1990 for discussion of various yeast promoters, markers, descriptions of yeast handling and culture procedures, and protocols for performing recombinant DNA procedures, screens and selections, etc.).

A wide variety of reporter genes may be used, provided that the particular DNA response element that provides UAS or enhancer function for the promoter region that is operably linked to the sequence encoding a detectable marker corresponds with (i.e., is bound by) the NR DNA binding domain that is used in the activator, such that the activator indeed is capable of activating transcription of the sequence encoding a detectable marker (e.g., when a DBD derived from GR is used, the appropriate DNA response element should contain one or more GREs). One of ordinary skill in the art will be able to select appropriate DNA response element/NR DBD pairs for creation of fusion gene constructs, to create such constructs using standard recombinant DNA techniques, and to test such constructs to ensure that they function as expected. Example 10 presents a strategy for generating rGR/NHR-25 LBD NR protein chimeras.

Numerous different detectable markers or functional portions thereof may be used, and one skilled in the art will recognize that a wide variety of reporter genes and associated detection methods can be used in the practice of the invention. Thus the methods disclosed herein are not limited to any specific reporter gene or genes. As used herein the term “detectable marker” includes selectable markers, e.g., markers that confer on yeast (or other cells) an ability to grow under particular conditions such as on media that lack one or more essential amino acids, or media that contain a toxic drug. Markers that produce a fluorescent, chemiluminescent, or colorimetric readout suitable for detection using an automated plate reader may be used. One skilled in the art will readily be able to select appropriate detection methods for any particular reporter gene. For example, if the reporter gene encodes a fluorescent protein, one skilled in the art will be able to select appropriate excitation wavelengths (e.g., near UV or blue light in the case of GFP), microscopes, fluorescence detectors, etc., for detection of the marker. Examples of reporter genes that can be used to monitor reporter gene expression include, but are not limited to, lacZ, chloramphenicol acetyltransferase (CAT), horseradish peroxidase, alkaline phosphatase, or active portions of any of these. Detection methods for these markers are well known in the art. Alternatively, the reporter may comprise a predetermined polypeptide sequence which can be recognized by a molecule such as an antibody. Such predetermined polypeptide sequences include, but are not limited to, epitope tags such as the HA tag, Myc tag, etc., for which monoclonal antibodies are commercially available. etc. In certain embodiments of the invention, detectable markers that can be assayed in microtiter well format are preferred.

Examples of selectable markers for use in yeast are well known in the art and are described, for example, in the references on yeast mentioned above and ref 71 in grant. Where a selectable marker is used, the identification of a compound as a modulator of an NR will be accomplished by identifying compounds that either enhance or reduce the viability of the cell or it's ability to reproduce. Methods for performing screens and selections in yeast are well known in the art. See for example, without intending to be limiting, M. Berg, K. Undisz, R. Thiericke, T. Moore, & C. Posten, Miniaturization of a functional transcription assay in yeast (human progesterone receptor) in the 384- and 1536-well plate format. Journal of Biomolecular Screening 5:71-76, 2000; I. G. Serebriiskii, G. G. Toby, & E. A. Golemis, Streamlined yeast colorimetric reporter activity assays using scanners and plate readers. Biotechniques 29:278-288, 2000; M. J. Garabedian & K. R. Yamamoto, Genetic dissection of the signaling domain of a mammalian steroid receptor in yeast. Molecular Biology of the Cell 3: 1245-1257, 1992. Additional references that discuss yeast two-hybrid screens include Walhout, A J, Boulton, S J, Vidal M, Yeast two-hybrid systems and protein interaction mapping projects for yeast and worm, Yeast, Jun 30;17(2):88-94, 2000; Fashena, S J, Serebriiskii, I., and Golemis, E A, The continued evolution of two-hybrid screening approaches in yeast: how to outwit different preys with different baits. Gene. May 30;250(1-2):1-14, 2000; Uetz, P and Hughes, RE, Systematic and large-scale two-hybrid screens. Curr Opin Microbiol. Jun;3(3):303-8, 2000. Selections and screens can be performed either in liquid culture or on solid media. Although two-hybrid screens are typically used to detect protein-protein interactions rather than protein-DNA interactions as in certain of the methods of the invention, one of ordinary skill in the art will perceive the similarities and be able to adapt vectors and screening and selection methodology as appropriate.

As will be appreciated by one of ordinary skill in the art, variants of any of the reporters described above may also be used, provided that such variants retain the features necessary for their detection. Such variants include polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs where in one or more amino acid residues is added to a terminal or medial portion of the polypeptides) and which share some or all properties of naturally-occurring forms, e.g., fluorescence, recognition by an antibody, etc.

(iii) Screens for Nematocides Using Sensitized Nematode Strains

The invention further encompasses the realization that nematode strains bearing mutations that reduce or eliminate the amount and/or activity of a xenobiotic sensing nematode NR provide a sensitized genetic background for screens designed to identify new anti-nematode agents or to identify agents that may act synergistically with modulators of a nematode xenobiotic sensing NR. As discussed above, in general, reducing or eliminating the amount and/or activity of a xenobiotic sensing NR leads to a reduction in the metabolism, biotransformation, and ultimate detoxification of a xenobiotic to which the NR is responsive (though as discussed above in certain situations such as where the active form of a compound is a metabolite, increasing the amount and/or activity of a xenobiotic sensing NR may lead to an increase in the effective amount of the xenobiotic). Such a reduction may cause various effects that increase sensitivity of the organism to the xenobiotic, e.g., by causing alterations in key pharmacokinetic parameters. Thus, for example, reducing or eliminating the activity of a xenobiotic sensing NR may lead to higher peak concentration and/or longer half-life of the xenobiotic within the nematode. This could make it possible to detect an effect (e.g., decreased viability, growth, reproduction, or feeding) that would otherwise not be detected and help to overcome limitations such as compound solubility, ability of the compound to enter the nematode or be absorbed, etc. In short, use of a sensitized genetic background for screening makes it possible to identify certain compounds that have anti-nematode effects which, for one or more reasons, would be “missed” in a screen performed using wild type animals.

A compound identified in a screen using a sensitized genetic screen may be suitable as a pharmaceutical and/or pesticide. Even if the compound itself is not suitable for use on wild type nematodes lacking the sensitized genetic background, the compound could serve as a valuable lead compound for the development of such agents. Once a lead compound having anti-nematode effects is identified, properties such as solubility and efficacy can be optimized, e.g., by using the lead compound as a basis for rational drug design or design of a combinatorial chemical library.

Thus the present invention provides a nematode having a sensitized genetic background, wherein the nematode has a mutation in a gene encoding a xenobiotic sensing NR. In certain embodiments of the invention the nematode is C. elegans. In certain embodiments of the invention the xenobiotic sensing nematode NR is a receptor encoded by the C. elegans nhr-8 gene. In certain embodiments of the invention the NR is encoded by the C. elegans nhr-48 gene. In certain embodiments of the invention the NR is encoded by the C. elegans daf-12 gene. Techniques for generating C. elegans mutants bearing a mutation in any particular gene of interest are well known in the art (see references mentioned above). Example 3 describes creation of a sensitized C. elegans strain having a mutation in the nhr-8 gene.

The invention provides methods of screening using a sensitized nematode. One such method is a method of identifying a compound with anti-nematode activity comprising steps of: (i) providing a sensitized nematode, wherein the sensitized nematode contains a mutation in a gene encoding a nuclear receptor; (ii) contacting the sensitized nematode with a test compound; and (iii) determining whether an indicator of nematode well-being, e.g., viability, growth, reproduction, or feeding of the sensitized nematode is decreased in the presence of the test compound, wherein a decrease in the indicator of nematode well-being in the presence of the test compound is an indication that the compound possesses anti-nematode activity.

In certain embodiments of the invention the method is performed using a nematode strain with a mutation in a gene that encodes a nuclear receptor in the NR1I/J subfamily. In certain embodiments of the invention the method is performed using a C. elegans strain with a mutation in the nhr-8 gene, such that the mutation reduces or eliminates the amount and/or activity of the NHR-8 protein. In other embodiments of the invention the method is performed using a C. elegans strain with a mutation in the nhr-48 gene, such that the mutation reduces or eliminates the amount and/or activity of the NHR-48 protein. In certain embodiments of the invention the method is performed using a C. elegans strain with a mutation in the daf-12 gene, such that the mutation reduces or eliminates the amount and/or activity of the DAF-12 protein. Mutants (e.g., double mutants) bearing a mutation in two or more of these genes may also be used. In addition, mutants bearing a mutation in a gene encoding a xenobiotic sensing NR and another gene (e.g., pgp-3) mutations in which confer increased toxin sensitivity may also be used. Methods for generating double mutants are well known in the art. See, e.g., Example 4 describing a nhr-8;pgp-3 double mutant. Although the phenotypes mentioned above (viability, growth, reproduction, and feeding) may be most useful to assess, other phenotypes such as movement, pharyngeal pumping, or defecation may be assessed in addition to or alternatively to these.

In certain embodiments of the invention rather than using a sensitized strain bearing a mutation in a gene encoding a xenobiotic sensing NR, the strain is sensitized by conducting the screen in the presence of a modulator (e.g., an antagonist) of a xenobiotic sensing NR or by conducting the screen using nematodes that have been pretreated with the modulator, such that the presence of the antagonist reduces the amount and/or activity of the NR. Thus the invention provides a method of identifying a compound with anti-nematode activity comprising steps of (i) providing a nematode; (ii) contacting the nematode with a modulator of a xenobiotic sensing nuclear, thereby generating a sensitized nematode; contacting the sensitized nematode with a test compound; and (iii) determining whether an indicator of nematode well-being, e.g., viability, growth, reproduction, or feeding of the sensitized nematode is altered in the presence of the test compound, wherein an alteration in the indicator nematode well-being (e.g., a decrease in viability, growth, reproduction, or feeding of the sensitized nematode) in the presence of the test compound is an indication that the compound possesses anti-nematode activity.

Any available modulator of a nematode xenobiotic sensing NR can be used to sensitize the nematode. For example, a modulator identified using a screen such as those described in the previous section may be employed. Modulators of vertebrate xenobiotic sensing NRs may be tested to determine whether they have similar effects on nematode xenbiotic sensing NRs.

The inventive screening methods involve testing compounds to determine their effect on a nematode phenotype such as viability, growth, reproduction, movement, feeding, etc. In a preferred embodiment of the invention the screen is adapted for the testing of multiple compounds in a high throughput fashion. Methods for growth of large quantities of C. elegans are well known in the art. (See, for example, Lewis, J. and Fleming, J., “Basic Culture Methods”, pp. 187-204 in Methods in Cell Biology, Epstein, H. and Shakes, D., (eds.), Vol. 48, Academic Press, San Diego, 1995, and references therein.) Methods for using C. elegans to perform high throughput screening are also known in the art (See, for example, Rand, J. and Johnson, C., “Genetic Pharmacology”, in Methods in Cell Biology, Vol. 48, pp. 187-204, 1995, and references therein.) For example, according to one procedure C. elegans are placed in liquid culture (e.g., in S medium or axenic medium), preferably in multi-well plates, e.g., 24 well plates, 96 well plates, 384 well plates, etc.

In certain embodiments of the invention approximately equal numbers of worms (e.g., between 5 and 25 worms per well for 96 well plates) are placed in each well. This can be accomplished, for example, by uniformly suspending worms in liquid prior to dispensing. In addition, commercially available machines, e.g., the Multidrop 384 from LabSystems and the COPAS machine, produced by Union Biometrica, Inc., (Somerville, Mass.), for more precise delivery of specified numbers of animals, can be used. The worms may be cultured for between 3 and 7 days, by which time the total population may range from several hundred to several thousand worms. Alternatively, a larger number of worms (e.g., several hundred to several thousand per well in a 96 well plate), may be added initially to the wells. Test compounds are added to each well. The compound may be added prior to, at approximately the same time as, or a variable amount of time after the worms are dispensed. The worms are cultured in the presence of the compound for a variable period of time, e.g., several hours to several days prior to assessment of the phenotype(s) of interest. Compounds that affect one or more phenotype(s) of interest are detected as appropriate for the particular phenotype. For example, to detect a compound that results in reduced viability, growth, reproduction, or feeding, wells in which a population of nematodes has failed to “clear” the bacterial food source may be identified. Such identification can be performed visually or by automated detection techniques that assess the turbidity, light absorbance characteristics, etc., in the wells. Although in preferred embodiments of the invention the screen is performed using C. elegans grown in liquid culture, the inventive screens can also be performed using C. elegans grown on solid medium (e.g., on agar plates), in which case compounds can be added to the poured plates and allowed to diffuse into the medium. Multiwell plates containing 96 wells are presently preferred, but the invention may be practiced using multiwell plates having smaller or larger numbers of wells such as are well known in the art. Note that for high throughput purposes the invention is not limited to using multiwell plates but may use any convenient format employing multiple vessels.

The worms can be cultured under various conditions before, during, and/or after the period of exposure to the test compounds. For example, the temperature, pH and/or osmolarity of the culture media can be varied. The worms can be cultured in the presence or absence of nutrients. One skilled in the art will be able to select varied environmental conditions compatible with life. Such conditions may influence various aspects of the phenotype(s) and/or metabolism of the compound, which may result in increased sensitivity for the screen. The screen can also be performed using C. elegans in different life stages, e.g., different larval stages including the dauer stage. Methods for generating synchronized populations of worms, including dauers, are well known in the art. As mentioned above, the alternative L3 stage known as dauer resembles, both phenotypically and biochemically, the infective L3 stage in many parasitic nematodes. In certain embodiments of the invention the screen is performed using dauer stage C. elegans that have been stimulated to resume development (e.g., by exposure to nutrients).

Compounds suitable for screening include small molecules, natural products, peptides, nucleic acids, etc. Sources for compounds include natural product extracts, collections of synthetic compounds, and compound libraries generated by combinatorial chemistry. Libraries of compounds are well known in the art. One representative example is known as DIVERSet , available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell-Based Assays”, Am. Chem Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc.

In general, compounds may be dissolved in any appropriate solvent, preferably a solvent that does not exert deleterious effects on C. elegans growth, development, etc. at the final concentration after addition of the compound solution to the nematode culture medium. In this regard it is noted that C. elegans can tolerate modest amounts of various common solvents (e.g., <1% dimethyl sulfoxide, <4% ethanol, <2% methanol) with little effect on growth or behavior. Thus the compound can be added to culture medium containing solvent (e.g., 1% dimethyl sulfoxide). In certain embodiments of the invention it may be desirable to employ lower solvent concentrations (e.g., when worms are grown in 384 well plates). Furthermore, the screen can also be performed by adding compounds to wells prior to the addition of worms, in which case the solvent can be allowed to evaporate before worms and culture media are added.

A range of concentrations of the compound may be tested for phenotypic effects. As is well known to one of ordinary skill in the art, virtually any compound can have deleterious effects on an organism if present at sufficiently high concentration. Preferred compounds exert their effects at concentrations practical for administration as therapeutic agents (e.g., at concentrations that do not cause unacceptable adverse effects in the host organism being treated) or as pesticides (e.g., at concentrations that do not cause unacceptable adverse effects on either nontargeted animal or plant species in the environment). Screens can be performed, for example, at relatively low concentrations such as <0.1 μg/ml, at higher concentrations such as 1 to 100 μg/ml, or at still higher concentrations, e.g., up to 1 mg/ml. In general, one of ordinary skill in the art will be able to select appropriate concentration ranges for testing, and the foregoing examples are not intended to be limiting. Of course the screen need not be performed in liquid culture but may employ worms growing on solid medium, e.g., agar plates. In this case the compound can be added to the agar, e.g., after pouring the plates, at any desired concentration.

Another parameter that may be varied at the discretion of the practitioner is the length of time between exposure to the compound and assessment of the phenotype(s) of interest. In this regard it is noted that compounds may become unstable after addition to the culture medium, in which case it may be desirable to perform the assessment relatively soon after exposing the nematodes to the compound. However, it is noted that the effect of a compound may not be immediately apparent. Thus it may be desirable to perform the assessment at a range of times following addition of the compound to the nematode culture medium.

It is noted that the inventive screens described in this section may be employed to identify new anti-nematode agents (e.g., compounds whose anti-nematode activity has hitherto not been known). However, the screens may also be employed to identify, among known anti-nematode agents, compounds that are likely to display synergistic effects with a modulator (e.g., an antagonist) of a nematode NR such as a nematode xenobiotic sensing NR.

Thus the invention provides a method of identifying a compound that acts synergistically with a modulator of a nematode nuclear receptor comprising steps of (i) providing a nematode, wherein the nematode contains a mutation in a gene encoding a nuclear receptor; (ii) contacting the nematode with the compound; and (iii) determining whether an indicator of nematode well-being in the nematode that contains the mutation in a gene encoding a nuclear receptor is altered relative to an indicator of nematode well-being in a nematode lacking the mutation in the gene encoding a nematode nuclear receptor, wherein the indicator of nematode well-being is assessed in the presence of the compound, and wherein an alteration in the indicator of nematode well-being in the nematode containing the mutation in a nematode nuclear receptor relative to the indicator of nematode well-being in a nematode lacking the mutation in the gene encoding a nematode nuclear receptor is an indication that the compound acts synergistically with a modulator of a nematode nuclear receptor. According to certain embodiments of the invention the indicator of nematode well-being is a decrease in viability, growth, reproduction, feeding, or movement and the alteration is a decrease in the indicator. According to certain embodiments of the invention the modulator is an antagonist of the NR.

A second method of identifying a compound that acts synergistically with a modulator of a nematode nuclear receptor comprises steps of (i) providing a nematode, wherein the nematode contains a mutation in a gene encoding a nuclear receptor; (ii) contacting the nematode with the modulator of a nematode nuclear receptor; and (iii) determining whether an indicator of nematode well-being, e.g., viability, growth, reproduction, or feeding of the nematode in the presence of the compound is altered relative to an indicator of nematode well-being in the presence of the compound, of a nematode that is not contacted with the modulator of the nematode nuclear receptor, wherein an alteration in the indicator of nematode well-being of the nematode contacted with both the modulator of the nematode nuclear receptor and the compound relative to the indicator of nematode well-being of a nematode contacted with the compound but not with the modulator of a nematode nuclear receptor is an indication that the compound acts synergistically with a modulator of a nematode nuclear receptor. According to certain embodiments of the invention the indicator of nematode well-being is a decrease in viability, growth, reproduction, feeding, or movement and the alteration is a decrease in the indicator. According to certain embodiments of the invention the modulator is an antagonist of the NR. In certain embodiments of the invention the NR is a nematode xenobiotic sensing NR, e.g., C. elegans NHR-8, NHR-48, or DAF-12.

(iv) Screens for Combinations of Compounds With Anti-Nematode Activity

The invention provides methods for identifying combinations of compounds with anti-nematode activity and compounds identified using the methods. In certain embodiments of the invention the combinations include a compound that modulates a nematode xenobiotic sensing NR. In certain embodiments of the invention the combination includes a compound that acts synergistically with such a modulator. One method of identifying a combination of compounds having anti-nematode activity comprises steps of: (i) providing a nematode; (ii) contacting the nematode with a first compound that modulates activity of a xenobiotic sensing nuclear receptor; (iii) contacting the nematode with a second compound; and (iv) determining whether an indicator of nematode well-being is altered in the presence of both the first compound and the second compound to a greater extent than in the presence of only the first compound or the second compound. According to certain embodiments of the method, the indicator of nematode well-being is the viability, growth, reproduction, or feeding of the nematode, and a greater decrease in the viability, growth, reproduction, or feeding of the nematode in the presence of the first compound and the second compound than in the presence of either the first compound or the second compound, is an indication that the combination of first and second compounds possesses anti-nematode activity. Other indicators of nematode well-being may also be used as mentioned above. In certain embodiments of the invention the first compound is an antagonist of a nematode xenobiotic sensing NR, e.g., a compound identified using a screen such as the yeast screen described herein.

The invention further provides a method of identifying a combination of compounds having anti-nematode activity comprising steps of (i) contacting a nematode with a first compound that modulates activity or amount of a xenobiotic sensing nuclear receptor; (ii) contacting the nematode with a second compound; and (iii) determining whether an indicator of nematode well-being of the nematode is altered in the presence of both the first compound and the second compound to a greater extent than in the presence of only the first compound or the second compound, wherein a greater alteration in the indicator of nematode well-being of the nematode in the presence of a combination of both first and second compounds than would result from adding the alteration that occurs in the presence of the first compound and the alteration that occurs in the presence of the second compound is an indication that the combination of compounds has synergistic anti-nematode activity. In certain embodiments of the invention the indicator of nematode well-being is viability, growth, reproduction, feeding, or movement and the alteration is a decrease in the indicator of nematode well-being.

In certain embodiments of the invention the nematode is C. elegans while in other embodiments of the invention the nematode is a parasitic nematode species. In certain embodiments of the invention the nematode xenobiotic sensing NR is NHR-8, NHR-48, DAF-12, or a parasitic nematode homolog, ortholog, or paralog thereof.

(v) Testing and Uses of Identified Compounds

In certain embodiments of the invention it is desirable to test compounds identified according to the methods described above (e.g., modulators of a nematode NR, combinations of such compounds, synergistic combinations of compounds) to determine their effects on viability, growth, development, infectivity, etc., of a parasitic nematode species. Direct effects (e.g., killing) on the target species can be observed. A number of in vitro culture systems and models for nematode parasitism are known in the art and can be used for these purposes. Example 12 in inventors provisional patent application U.S.S.No. 60/263,081, filed Jan. 18, 2001, entitled “SCREENS AND ASSAYS FOR AGENTS USEFUL IN CONTROLLING PARASITIC NEMATODES” and copending U.S national application U.S. Ser. No. 10/051,644 entitled “SCREENS AND ASSAYS FOR AGENTS USEFUL IN CONTROLLING PARASITIC NEMATODES” filed Jan. 18, 2002, describe the process of testing a candidate compound to determine its effects on the release of ASP-1 by A. caninum and the resumption of larval feeding and development upon stimulation (an in vitro model for parasitism). Candidate compounds can also be tested, for example, by administering them to a host (e.g., an animal or plant) either before or after infection of the host by a parasitic nematode and assessing the ability of the compound to modify the course of infection, e.g., to reduce the incidence or severity of infection, to reduce the parasite burden, to reduce the amount of a second agent needed to control the infection (i.e., to synergize with another anti-nematode agent), etc. Candidate compounds can also be tested by comparing the amount of damage caused by a plant parasitic nematode to a susceptible plant species grown in the presence of the compound versus the amount of damage caused by the same number of nematodes in the absence of the compound. In general, methods well known in the art for evaluating the efficacy of anti-nematode therapeutic agents and standard methods for evaluating anti-nematode agricultural agents may be used. For animal and human parasitic nematodes such methods are described, for example, in Conder G A, Campbell W C. “Chemotherapy of nematode infections of veterinary importance, with special reference to drug resistance”, Adv Parasitol, 35:1-84, 1995; Campbell W C, Rew R S, eds., Chemotherapy of Parasitic Diseases, New York: Plenum Press, 1986. Methods applicable to plant parasitic nematodes are described in Hague N G M, Gowen S R. “Chemical control of nematodes”, Chapter 5 in Brown R H, Kerry B R, eds. Principles and Practice of Nematode Control in Crops, Sydney: Academic Press, pp.131-178, 1986.

Compounds that modulate (e.g., antagonize) a nematode xenobiotic sensing NR may be used as therapeutic agents, as pesticides, or in any context in which it is desired to inhibit nematode growth, feeding, and/or reproduction. If the compound was identified by screening natural product extracts, the active component can be determined using standard techniques. If the compound was identified by screening a library appropriate standard techniques may be used to determine the identity of the compound. In many cases this is entirely straightforward since individual compounds are distributed in individual vessels, microtitre plates, etc., that comprise the library. In other cases, (e.g., for a combinatorial library), identifying the compound may involve decoding tags to determine a synthetic route, etc. One of ordinary skill in the art will be able to select and apply appropriate techniques to identify the compound. Note that identifying the compound is not a requirement in certain embodiments of the invention. For example, if a component of a natural product extract modulates (e.g., antagonize) a nematode xenobiotic sensing NR, the extract or an active fraction thereof may be employed without identifying the active component(s).

Potential pharmaceutical candidates can be evaluated for various physicochemical properties that may influence solubility and/or absorption. (See, for example, Blake, J., “Chemoinformatics—predicting the physicochemical properties of ‘drug-like’ molecules', Curr. Op. Biotechnol., 11: 104-107, 2000, and references therein.) Drug candidates are typically screened for cytotoxicity and evaluated in a standard set of cell-based assays well known in the pharmaceutical arts (e.g., to examine metabolism). For therapeutic purposes, pharmaceutical compositions comprising an active compound or compounds can be formulated according to methods well known in the pharmaceutical arts for delivery by any appropriate route (e.g., oral, intravenous, transdermal, etc.) in any appropriate vehicle or carrier. Methods for determining an appropriate formulation and route of administration are known in the art. Determining an appropriate dose will involve trials (initially in animals and ultimately in humans in the case of human therapeutics) to evaluate the effect of a candidate therapeutic agent on the course of nematode infection and/or to evaluate the adverse effects (if any) of the agent. The pharmaceutical compositions can be used either alone or in combination with other anti-nematode agents.

For use as pesticides, compounds that modulate (e.g., antagonize) a nematode xenobiotic sensing NR can be formulated for application using conventional techniques, e.g., as a liquid or powder typically for delivery to the plant and/or to the soil at or near the time of planting, during irrigation, etc. The compounds can be combined with an agriculturally suitable carrier, e.g., a liquid such as water,, alcohol, or other organic solvents or oils, or a powder such as talc, clay, or other silicate to produce an agriculturally appropriate anti-nematode agent. The compounds may also be formulated for delivery as fumigants. The compounds can be used either alone or in combination with other anti-nematode agents. The anti-nematode agents of the invention need not be applied directly to the plant or to its roots but may be applied in the vicinity of the plant, e.g., within a meter from the location of the plant. The agents can also be applied to a seed from which a plant is to be grown. For example, seeds can be coated with the agents prior to planting.

It is noted that in certain embodiments of the invention it is desirable to employ a combination of compounds comprising a modulator of a parasitic nematode xenobiotic sensing NR (e.g., a compound identified according to the inventive screening methods described above) and a second (or more than one) compound that exhibits synergistic activity with the modulator. The second compound can comprise a known anti-nematode agent or a newly identified anti-nematode agent identified according to the methods described herein.

In certain embodiments of the invention compounds that modulate a nematode xenobiotic sensing NR are expressed by transgenic plants or animals, so that the transgenic plant or animal exhibits increased resistance against nematode infection and/or damage. Most typically, an inhibitory compound suitable for expression in a transgenic organism is a nucleic acid, peptide, or polypeptide, although it is within the scope of the invention to generate transgenic organisms that express or overexpress small molecules, lipids, etc., e.g., by introducing genes that encode enzymes involved in the synthesis of such compounds into the organism. Methods of generating transgenic plants or animals are well known in the art. The transgene may comprise endogenous sequences, exogenous sequences, or a combination of the two. The transgene will typically comprise regulatory elements capable of directing gene expression within a particular organism or cell type in which the transgene is to be expressed (e.g., a promoter) operably linked to DNA sequences that encode an inhibitory compound, an enzyme involved in the synthesis of such a compound, etc. In addition to a promoter, other regulatory elements may also be included in the transgene, e.g., splice sites, polyadenylation sites, transcriptional terminators, etc. In some cases increased expression of an inhibitory compound in a transgenic organism may be achieved by “knocking out” expression of an endogenous gene in the organism according to methods well known in the art. For example, such an endogenous gene may be a gene whose product normally inhibits expression of an endogenous inhibitor of a nematode xenobiotic sensing pathway.

(vi) Use of C. elegans Strains Bearing a Mutation in a Gene Encoding a Xenobiotic Sensing NR as a Sensitized Background to Identify Genetic Targets for Known Compounds.

According to one aspect of the invention, a C. elegans strains bearing a mutation in a gene encoding a xenobiotic sensing NR (e.g., a mutation in the nhr-8, nhr-48, or daf-12 gene is used as a sensitized background to identify genetic targets for known compounds (e.g., anti-nematode agents). One such method of identifying a genetic target for a compound comprises the steps of: (i) contacting a population of starting strain nematodes with the compound, wherein the starting strain nematodes have a mutation in a gene encoding a xenobiotic sensing nuclear receptor, and wherein contacting a starting strain nematode with the compound causes a detectable phenotype in the nematode; (ii) mutagenizing the population of starting strain nematodes; and (iii) identifying a mutant that is resistant to the compound, wherein the resistance is manifested by an alteration in the detectable phenotype in the mutant. The method can further include the step of cloning the gene that is mutated in the mutant identified in the identifying step, thereby identifying a genetic target for the compound. According to certain embodiments of the invention the detectable phenotype is an indicator of nematode well-being, e.g., viability, growth, reproduction, feeding, or the like. For example, contacting the starting strain nematode with the compound may cause a decrease in viability, growth, reproduction, movement, or feeding, in which case resistance to the compound is manifested by greater viability, growth, reproduction, movement, or feeding, of the mutant in the presence of the compound relative to viability, growth, reproduction, movement, or feeding, of the starting strain in the presence of the compound. In this case the method comprises steps of (i) contacting a population of starting strain nematodes with a compound, wherein the nematodes have a mutation in a gene encoding a xenobiotic sensing nuclear receptor, and wherein contacting a nematode with the compound causes a decrease in viability, growth, reproduction, movement, or feeding, of the nematode; (ii) mutagenizing the population of nematodes; and (iii) identifying a mutant that is resistant to the compound, wherein the resistance is manifested by increased viability, growth, reproduction, movement, or feeding, of the mutant in the presence of the compound relative to the viability, growth, reproduction, movement, or feeding, of the starting strain in the presence of the compound.

Genetic screens for mutants whose response to a compound is either reduced (suppressed) or enhanced can reveal the identity of the target of the compound. Knowing the identities of potential targets (e.g., the amino acid sequence of protein targets, nucleotide sequence of gene targets, etc.) allows the use of tools such as molecular drug design, screens employing libraries particularly adapted for a given target, etc. Potential targets may include genes and proteins that are members of families that have been studied in other organisms (or in nematodes) and/or for which structural information, information about interacting molecules, etc. is already known.

Given their attractiveness as potential targets for development of anti-nematode agents, it is of considerable use to identify genes and proteins that are targets for anti-nematode agents. According to one line of reasoning, a strain that bears a mutation in a gene that encodes or comprises a target for an anti-nematode agent will be resistant to that agent. Using a sensitized strain, e.g., a sensitized C. elegans strain, to identify genetic targets offers a number of advantages. As discussed above, procedures for performing genetic manipulation in C. elegans are well known, in contrast to the situation for other nematodes. However, it may be the case that a compound that exerts anti-nematode effects in certain nematode species of interest does not exert such effects on C. elegans (or does so only at concentrations higher than that which can practically be achieved). In such a situation it would not be possible to perform a screen for resistant C. elegans mutants since even wild type worms are resistant. However, such a screen could be performed in a sensitized C. elegans strain that displayed sensitivity to the compound due to either to a sensitized genetic background (e.g., a mutation in a gene encoding a xenobiotic sensing NR) or to the presence of a compound (e.g., an antagonist of a xenobiotic sensing NR) that causes reduced metabolism, biotransformation, etc., of the anti-nematode compound for which a genetic target is sought. Thus use of a sensitized strain may make it possible to use C. elegans to screen for genetic targets for compounds to which wild type C. elegans is normally resistant. In addition, use of a sensitized strain would potentially allow use of lower concentrations of the anti-nematode compound in the screen. Furthermore, use of a sensitized strain may make the difference between resistant mutants that have a mutation in a gene encoding or comprising a target for the compound and worms that lack such a mutation more evident, thus improving the sensitivity of the screen.

In a preferred embodiment of the genetic screen for an anti-nematode target, hermaphrodites from a C. elegans strain are mutagenized (e.g., using a chemical mutagen such as EMS or ENU, irradiation, transposon-mediated mutagenesis, or any other mutagenesis technique). Their progeny (F1) are screened for dominant mutations, and the following generation (F2) is screened for recessive mutations. Mutants displaying resistance to an anti-nematode compound are identified. Mutants identified as described above are preferably backcrossed (crossed to the starting strain) several times to eliminate extraneous mutations that may have been caused by the mutagen (i.e., mutations not reducing or affecting expression of the gene of interest). In certain embodiments of the inventive method mutations are mapped (e.g., using conventional two and three-factor crosses as described in Sulston and Hodgkin, Methods, in The Nematode Caenorhabditis elegans, W. B. Wood, ed., pp. 587-606, 1988; using polymorphic sequence tagged sites as described in Williams, B., Genetic Mapping with Polymorphic Sequence Tagged Sites in Methods in Cell Biology, Vol. 43, referenced above, using single nucleotide polymorphisms (SNPS) as described in Jakubowksi and Kornfeld, A local, high density, single nucleotide polymorphism map used to clone Caenorhabditis elegans cdf-1, Genetics 153: 743-752, 1999, or using any other available mapping technique.). Complementation tests are generally performed to determine the number of genes represented. In preferred embodiments of the invention the phenotypes of expression mutants are characterized with respect to the expression pattern of the gene of interest and also with respect to any of a variety of phenotypes including, for example, dauer formation, hatching, molting, or feeding.

In certain embodiments of the invention, the wild type version of the mutated gene is cloned. Methods for cloning C. elegans genes are well known in the art. Typically, after mapping a gene to a chromosomal region using, for example, two and three-factor crosses, cosmids spanning the region are injected into mutant worms, and the ability of a particular cosmid to complement the mutant phenotype is taken as an indication that the wild type gene is located on that cosmid. Individual genes located on the cosmid can then be individually assessed for their ability to complement the mutant phenotype. Genes identified in the inventive genetic screen and their encoded proteins are targets for the development of additional anti-nematode compounds or the optimization of known compounds.

(vii) Use of Sensitized C. elegans Strains to Enhance Compound Identification in Model Screening Systems and to Identify Genetic Targets for Known Compounds

As described above, the inventors have recognized that sensitized C. elegans strains, e.g., strains bearing a mutation in a xenobiotic sensing NR, are useful in the identification of new anti-nematode compounds and in the identification of new genetic targets for known compounds. More broadly, the inventors have recognized that a sensitized strain could have similar applications as a sensitized background for performing screens in a wide variety of other contexts and for identifying as yet unknown genetic targets. As is well known in the art, numerous genes and proteins present in C. elegans have homologs in other organisms, and numerous biochemical pathways are conserved between nematodes and other organisms, including humans. In addition, genes from other organsims (and their encoded proteins) may often substitute functionally in such pathways. These observations have led to the development of numerous nematode model systems useful for screening for pharmaceuticals, insecticides, etc. For example, nematode screening strains that are useful for screening for compounds that interact with the insulin receptor signalling pathway have been developed. Nematode model systems for screening for compounds that may be useful in the treatment of Huntington's disease have been developed. (See, e.g., Link, E. M., et al., Therapeutic target discovery using Caenorhabditis elegans, Pharmacogenomics 2000 May;1(2):203-17; Culetto, E., et al., A role for Caenorhabditis elegans in understanding the function and interactions of human disease genes, Hum Mol Genet. 2000 Apr 12;9(6):869-77). While many nematode model systems are designed to screen for compounds useful in treatment of human disease, systems useful to screen for compounds useful as treatment for animal disease, systems useful to screen for compounds useful as insecticides or other types of pesticides, etc., have also been developed. The phenotype of interest that serves as a “readout” for such a system may be any of a wide variety of phenotypes including, but not limited, to, viability, movement, reproduction, defecation, dauer formation, hatching, molting, feeding, or the expression of a particular gene or protein, e.g., a reporter gene or protein.

Many screening strains developed for use in model screening systems contain genetic alterations. For example, such strains may have mutations that increase or inhibit the expression or activity of a C. elegans gene. Such strains may contain transgenes, e.g., genes from another organism, genes encoding reporter proteins, etc. These screening strains may readily be modified to convert them into a sensitized strain as described herein in any of a number of ways. For example, the screening strain may be crossed with a sensitized strain that has a mutation in a gene encoding a nematode NR (e.g., NHR-8, NHR-48, or DAF-12). F2 progeny that contain both the mutation in the gene encoding a nematode NR and that display the other features of the screening strain may be selected according to methods that are well known in the art. Alternatively, the screening strain may be modified to increase its sensitivity by inactivating a nematode NR using RNAi. The sensitized screening strain may be employed in a similar fashion to that envisioned for the screening strain.

Since a sensitized screening strain may exhibit increased sensitivity to any of a wide variety of compounds that may be tested using such a model system, the use of a sensitized genetic background in the context of such model systems offers an enhanced opportunity to identify active compounds. For example, the use of a sensitized strain may result in achievement of a higher effective concentration of a test compound by reducing or inhibiting the metabolism of the test compound. Thus a test compound that would be effectively metabolized and eliminated in a strain having wild type xenobiotic metabolizing capability, and which would therefore not be identified as an active compound in the screen, would be more readily identified in a strain that exhibited reduced xenobiotic metabolizing capacity.

Just as sensitized screening strains are useful to enhance identification of new compounds in the context of existing or future nematode model screening systems, such sensitized screening strains are useful in the identification of additional target genes, e.g., target genes that encode proteins that function in the same pathway as that being modeled in the screening strain or genes that encode proteins that affect the pathway or the phenotype exhibited by the screening strain. Genetic screens for additional target genes may be performed as described in the previous section. For example, in a preferred embodiment of the genetic screen for a new genetic target, hermaphrodites from a C. elegans strain are mutagenized (e.g., using a chemical mutagen such as EMS or ENU, irradiation, transposon-mediated mutagenesis, or any other mutagenesis technique). Their progeny (F1) are screened for dominant mutations, and the following generation (F2) is screened for recessive mutations. Mutants displaying an enhanced or exaggerated phenotype in response to a particular compound relative to the phenotype of the starting strain in response to the particular phenotype are identified. The nature of the phenotyp and the phenotypic response will vary depending upon the particular model system and/or screening strain. Mutants identified as described above are preferably backcrossed (crossed to the starting strain) several times to eliminate extraneous mutations that may have been caused by the mutagen (i.e., mutations not reducing or affecting expression of the gene of interest). In certain embodiments of the inventive method mutations are mapped (e.g., using conventional two and three-factor crosses as described in Sulston and Hodgkin, Methods, in The Nematode Caenorhabditis elegans, W. B. Wood, ed., pp. 587-606, 1988; using polymorphic sequence tagged sites as described in Williams, B., Genetic Mapping with Polymorphic Sequence Tagged Sites in Methods in Cell Biology, Vol. 43, referenced above, using single nucleotide polymorphisms (SNPS) as described in Jakubowksi and Kornfeld, A local, high density, single nucleotide polymorphism map used to clone Caenorhabditis elegans cdf-1, Genetics 153: 743-752, 1999, or using any other available mapping technique.). Complementation tests are generally performed to determine the number of genes represented. In preferred embodiments of the invention the phenotypes of expression mutants are characterized with respect to the expression pattern of the gene of interest and also with respect to any of a variety of phenotypes including, for example, dauer formation, hatching, molting, or feeding.

In certain embodiments of the invention, the wild type version of the mutated gene is cloned. Methods for cloning C. elegans genes are well known in the art. Typically, after mapping a gene to a chromosomal region using, for example, two and three-factor crosses, cosmids spanning the region are injected into mutant worms, and the ability of a particular cosmid to complement the mutant phenotype is taken as an indication that the wild type gene is located on that cosmid. Individual genes located on the cosmid can then be individually assessed for their ability to complement the mutant phenotype. Genes identified in the inventive genetic screen and their encoded proteins are targets for the development of additional compounds active in the model screening system and in the biological state (e.g., disease state or organism) that it is designed to mimic, or for the optimization of known compounds.

It is noted that the invention is not limited to the embodiments described herein and in the examples presented below and that various changes and modifications may be made by those of ordinary skill in the art without departing from the scope and spirit of the appended claims.

### EXAMPLES

### Example 1

### Sequence Analysis of NHR-8 and Other Nuclear Receptors Shows that NHR-8 is a Member of the VDR/PXR/CAR (NR1I/J) NR Subfamily

Materials and Methods

Comparative Sequence Analysis

Multiple sequence alignments were generated with the PILEUP program of the GCG software package (Wisconsin Package Version 10.1, Genetics Computer Group, Madison, Wis.), using a modified scoring matrix optimized for alignment of NR sequences [10], a gap creation penalty of 12, and a gap extension penalty of 2. The neighbor-joining tree and bootstrap analyses were completed using the CLUSTAL X software with default parameters [34]. The highly variable N-terminal A/B domain sequences were omitted from the analysis, as were the large hinge domains of NHR-8, DHR96, and DAF-12, by deleting these segments from the sequence files used in the multiple sequence alignment from which the tree was derived. Maximum parsimony analysis was performed using the PAUPsearch feature of the GCG software package (Wisconsin Package Version 10.1, Genetics Computer Group (GCG), Madison, Wis.) with default parameters and 1000 bootstrap replicates.

GenBank accession numbers for the sequences included are: C. elegans DAF-12 (AF136238); C. elegans NHR-8 (AF083226); C. elegans NHR-48 (Z79604); chicken VDR (AF011356); chicken CXR (AF276753); Drosophila E75A (X51548; Drosophila EcR (M74078); Drosophila Ftz-F1 (M63711); Drosophila HNF4 (pir:S36218); Drosophila HR3 (M90806); Drosophila HR38 (X89246); Drosophila HR96 (U36792); Drosophila SVP (M28863); Drosophila TLL (M34639); Drosophila USP (X52591); Human CAR (Z30425); Human COUP TFI (X16155); Human ER (X03635); Human ERRI (X51416); Human FXR (U68233); Human GCNF (U64876); Human GR (M10901); Human HNF4 (X76930); Human LRH1 (U93553); Human NGFI-B (L13740); Human PPARγ (Z30972); Human PXR (AF0610561); Human RARγ (M24857); Human Rev-Erbα (X55066); Human RORα (U04898); Human RXRα (X52773); Human SF1 (U76388); Human TLL (Y13276); Human VDR (X67482); Mouse PXR (AF031814); T. molitor HR4 (AJ005685); Xenopus BXR (X75163); Xenopus VDR (U91846).

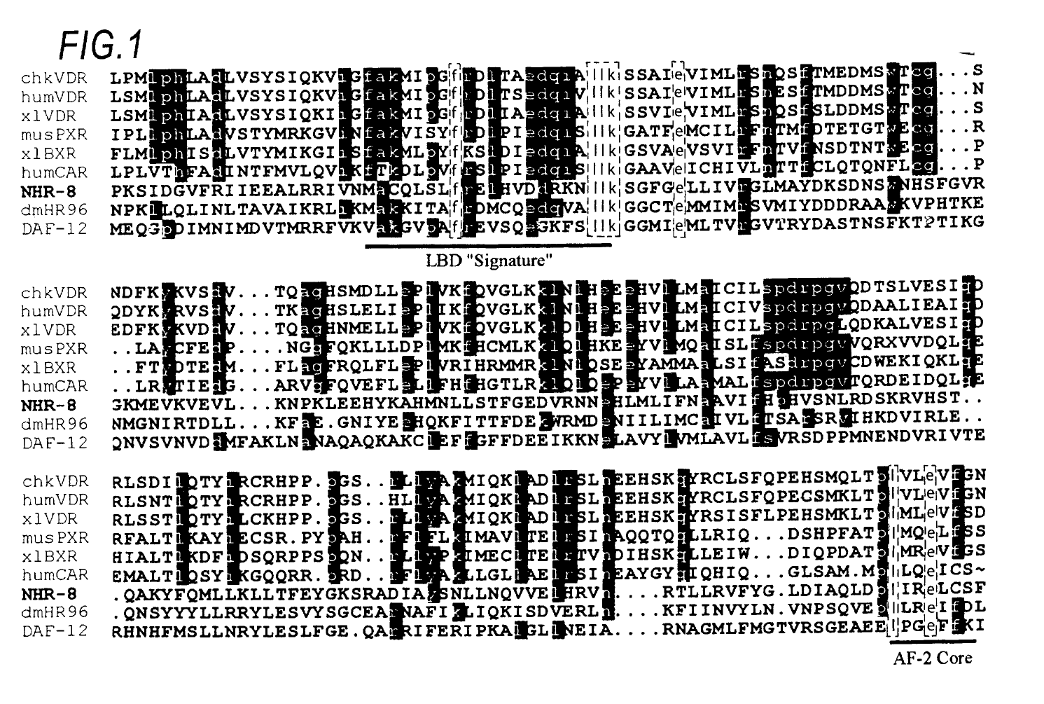

The initial placement of NHR-8 in the NR subfamily containing PXR and CAR was based upon a comparative analysis of only DNA binding domain (DBD) sequences [10, 13]. However, the ligand binding domain (LBD) of NHR-8 also shows homology to other NRs in the LBD signature motif and strong transactivation motif AF-2 (FIG. 1). Therefore, to confirm the placement of nhr-8 into the NR1I/J subfamily, the analysis was expanded to include both the DBD and LBD sequences, comparing NHR-8 to representatives of the major NR subfamilies defined in a comprehensive analysis of NR evolution [21].

Results

FIG. 1 shows sequence conservation of the ligand binding domain among the NR1I/J subfamily members, including NHR-8. 100% identity is shown by the light shading and 60% or greater consensus in dark shading. Shaded letters are lower case for easier reading. Species abbreviations are: no designation, C. elegans; dm, Drosophila melanogaster; chk, Gallus gallus (chicken); hum, Homo sapiens; mus, Mus musculus; and xl, Xenopus laevis. Sequences included are identified in the Materials and Methods. The “LBD signature” and “AF-2 core” motifs are marked as in Wurtz et al. [44]. These results further support inclusion of NHR-8 in the NR1I/J subfamily.

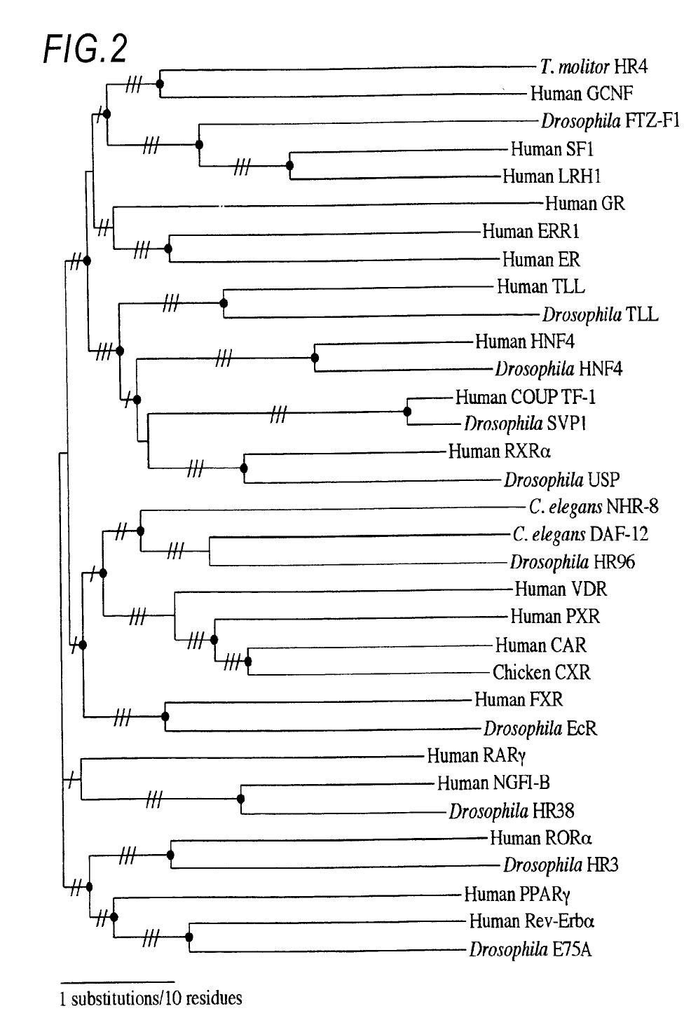

FIG. 2 is a neighbor-joining tree of selected conserved NR sequences that was generated with CLUSTAL W as described in Materials and Methods. Bootstrap values from 1000 replicates are represented by hatch marks on the supporting branches: (/) 50-79%, (//) 80-94%, and (///) 95-100%. Branch-points marked with dots were preserved in a maximum parsimony analysis of the same sequences. Sequences included are identified in Materials and Methods. NHR-48 was not included as it does not contain recognizable LBD sequences. These results further support the conclusion that NHR-8 is a member of the NR1I/J subfamily of NRs.

### Example 2

### Expression Pattern of nhr-8

Materials and Methods

Northern Analysis

For the developmental Northern analysis, polyA+ mRNA was prepared as described from staged cultures of the wild type strain N2 [10]. Total RNA was prepared from mixed stage cultures of N2 and nhr-8(ok186) using Trizol reagent as described [10]. Northern blots were prepared and probed using standard protocols [35, 36]. Each lane contained 2 μg of poly(A)+ RNA. The myo-1 pharyngeal myosin and nhr-48 mRNAs were detected on the same blot to control for mRNA loading and integrity.

RNA sizes were determined using the Millenium markers from Ambion (Austin, Tex.). A full length, ³²P-labeled, nhr-8 antisense riboprobe was prepared from the cDNA clone cm14e12 using the Strip-EZ RNA kit from Ambion (Austin, Tex.). Probes for pgp-3 and ama-1 were similarly prepared from PCR amplicons of the yk315f11 and yk481g3 cDNA clones, respectively (phage stocks were gifts from Dr. Yuji Kohara, National Institute of Genetics, Japan). A myo-1 riboprobe was produced from a myo-1 cDNA [37] cloned into pBlueScript.

nhr-8::GFP Transgenic Reporter Strains

An nhr-8::GFP translational fusion gene was constructed in the vector, pPD95.67 (gift from A. Fire, Carnegie Institute of Washington) by a two-step PCR cloning strategy [38] using primers EL-11 and EL-12. nhr-8 sequences were amplified from the cosmid F33D4, generously provided by A. Coulson (Sanger Center, Cambridge, England). The fusion gene contains ˜6.8 kb of genomic sequence upstream of the predicted translation start, the nhr-8 transcription and translation start sites, and 49 nhr-8 codons fused in frame to GFP coding sequences. The wild type C. elegans strain N2 [39] was transformed by microinjection of 20 μg/ml nhr-8::GFP fusion plasmid and 100 μg/ml of the marker plasmid pRF-4 [40]. A total of five independently isolated nhr-8::GFP fusion plasmids were used to generate transgenic nematode strains. All transgenic strains exhibited identical GFP activity.

Results

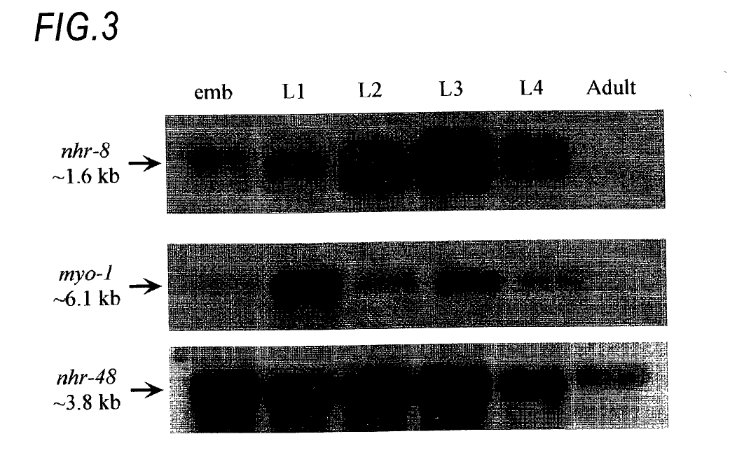

The inventors have shown that nhr-8 mRNA is expressed from embryo through the L4 larval stage. Expression for nhr-8 was first demonstrated by an expressed sequence tag (EST) project associated with the C. elegans genome sequencing project [22], and the sequence of the nhr-8 cDNA clone (cm14e12) thus identified was subsequently completed [10]. A developmental Northern blot probed with cm14e12 sequences detects a ˜1.6 kb mRNA in embryos and the four larval stages is shown in FIG. 3. nhr-8 mRNA was not detected in adults, suggesting that a relatively low level of nhr-8 mRNA is present in adults, though reprobing the blot with nhr-48 sequences confirmed the presence of intact RNA in this lane.

The inventors have shown that the nhr-8 promoter is active in the intestine. To determine the likely spatial expression pattern of nhr-8, GFP production from a reporter gene containing GFP coding sequences fused in frame to the third nhr-8 exon was examined. FIG. 4A is a schematic depicting the structure of the nhr-8::GFP transgene. FIG. 4B shows a differential interference contrast (DIC) micrograph of a transgenic larva (arrows) and a wild type larva (arrowhead), and FIG. 4C is an epifluorescence micrograph of the animals in FIG. 4B. In both FIGS. 4A and 4B the arrows mark the anterior/posterior extent of the intestine of the transgenic larva. As shown in FIG. 4B (and in data not shown), transgenic animals bearing the reporter express GFP exclusively in intestinal cells, beginning in embryos shortly after cellular differentiation and continuing throughout the life of the transgenic animals. The expression of the reporter in the adult stage contrasts with the Northern analysis, which did not detect nhr-8 mRNA in adults. This discrepancy could reflect either a low level of nhr-8 mRNA in the adult, below the sensitivity of the Northern analysis, or perdurance of GFP produced during the last larval stage.

The expression of the nhr-8::GFP reporter in the intestine suggests that nhr-8 plays a role in some aspect of gut function. The C. elegans intestine performs three major functions. First and foremost is digestion and absorption of nutrients. The other two functions, which are performed by hepatic tissues in vertebrates, are yolk production [23] and the detoxification and/or export of ingested and endogenous toxic substances [19, 20, 24]. nhr-8 could be involved in any or all of these functions.

### Example 3

### Construction and Analysis of the nhr-8(ok186) Deletion Allele and Mutant

Materials and Methods

Oligonucleotide Primer Sequences

Primers EL-11 and EL-12 were designed as described for PCR-mediated GFP tagging [38]. NR8 primers were designed by the C. elegans Gene Knock-out Consortium according to standard methods. pgp-3 primers were designed to detect the pgp-3(pk18) allele [20]. Sequences of the primers are presented below in a 5′-3′ direction.

| | | |
|---|---|---|
| EL-11: | GGA TGC ATG CAA CAA CAA TCA AAA CCA TAG | (SEQ ID NO:1) |
| EL-12: | GGC CAA TCC CGG GGA TCC TCT AGA CAA GAA TCA GAG AAA GGA CA | (SEQ ID NO:2) |
| EL-15: | AAT TAA CCC TCA CTA AAG GTC AAA GAC TCA CCG CAT ACG | (SEQ ID NO:3) |
| NR8-IL2: | GTA CTG ATC GTT GCC GGA TT | (SEQ ID NO:4) |
| NR8-IR2: | GCA ATG TCA GCA CGT GAT TT | (SEQ ID NO:5) |
| NR8-EL2: | CGA GGA TTT TTC CCA CAA AA | (SEQ ID NO:6) |
| NR8-ER2: | TTC ACA CGA TGA AGC TCG AC | (SEQ ID NO:7) |
| T7: | TAA TAC GAC TCA CTA TAG GG | (SEQ ID NO:8) |
| pgp3EL: | CTG TGC TTA TTG GAA CTA | (SEQ ID NO:9) |
| pgp3IR: | ACA AAG TGC ATC GTA GTA | (SEQ ID NO:10) |
| pgp3ER: | TGG GCA ATA ACT ACA CAA | (SEQ ID NO:11) |
| pgp3IL: | ATA TTT TCA AGG TCA TCG | (SEQ ID NO:12) |

nhr-8(ok186) Genetic and Molecular Analysis

A mutagenized population of nematodes containing animals bearing the nhr-8(ok186) allele was identified in a PCR-based screen by the C. elegans Knock Out Consortium (see Web site having URL elegans.bcgsc.bc.ca/knockout.shtml) under the direction of the inventors. nhr-8(ok186) homozygotes were recovered using PCR analysis of the single parent of a clonal population using nested primers (NR8 primers described above) to detect the deletion allele according to the strategy described in [41]. To determine the lesion in nhr-8(ok186), gel-purified PCR amplicons were sequenced at the University of Georgia Molecular Genetics Facility (Athens, Ga.).

Before performing phenotypic analyses, the nhr-8(ok186) mutant strain was outcrossed four times by mating wild type males to homozygous nhr-8(ok186) hermaphrodites. The resulting heterozygous males were mated to unc-22(s7) homozygous hermaphrodites [strain BC23, 42] to yield nhr-8(ok186)/unc-22(s7) heterozygotes. nhr-8(ok186) homozygotes were recovered from the non-Unc progeny of nhr-8(ok186)/unc-22(s7) hermaphrodites, using PCR to detect the deletion allele. The final outcrossed strain was designated AE501.

nhr-8(ok186); pgp-3(pk18) animals were obtained by crossing pgp-3(pk18) males [strain BC23, 42] to homozygous nhr-8(ok186) hermaphrodites and testing the progeny of individual F2 animals for the presence of the deletion alleles by PCR using the NR8 primers to detect nhr-8(ok186) and the pgp3 primers to detect pgp-3(pk18). The double mutant strain was designated AE505.

Results

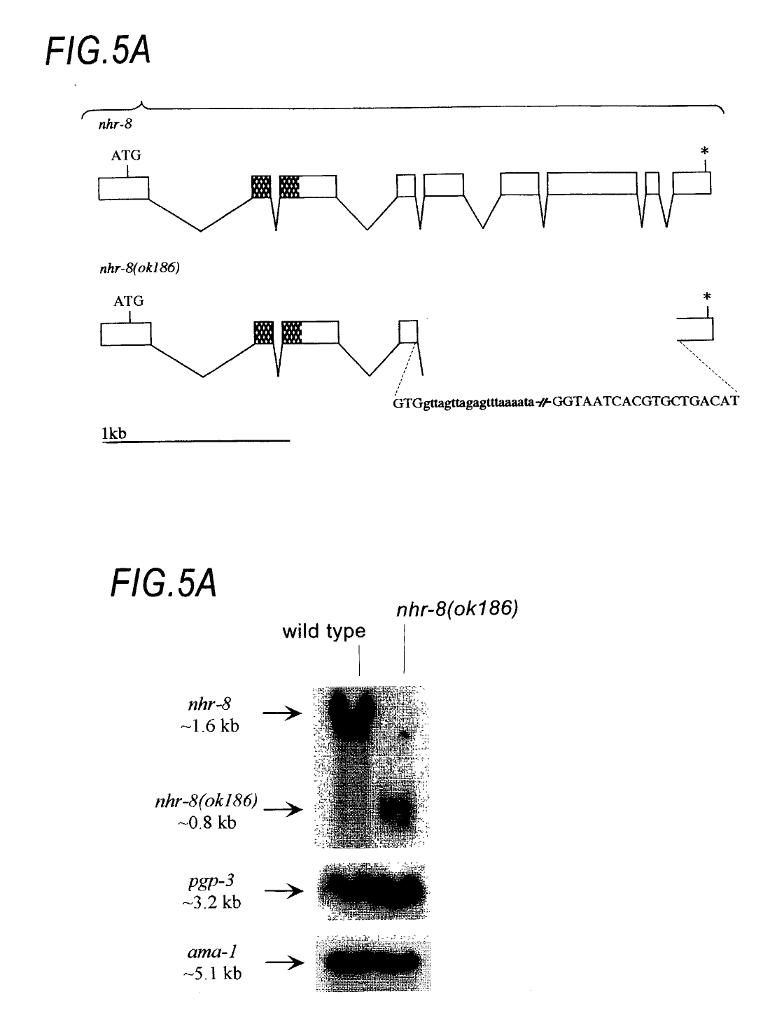

To determine the extent of the nhr-8(ok186) deletion, PCR amplicons of the nhr-8 genomic region were sequenced. The lesion in nhr-8(ok186) removes 1.3 kb with the 5′ breakpoint lying in the fourth predicted intron and the 3′ breakpoint in the last exon. Thus 4 exons and the 3′ splice junction of the last intron are removed. FIG. 5A shows the structure of the wild type nhr-8 locus and the nhr-8(ok186) deletion allele. The DBD is designated by the hatched box. The stop codon is denoted by *. The nhr-8(ok186) sequence is indicated with exon sequence in capital letters and intron sequence in lower case letters. The sequences encoding the LBD are completely removed, but those encoding the DBD remain intact.

Northern analysis (performed as described in Example 2) reveals that a stable, truncated RNA is produced from the nhr-8(ok186) locus as shown in FIG. 5B. Total RNA was purified from mixed stage cultures of wild type (Lane 1) and nhr-8(ok186) (Lane 2) animals. 30 μg of total RNA were loaded in each lane. An nhr-8 probe detects a correctly sized wild type transcript and an nhr-8(ok186) transcript consistent with transcription from the mutant locus. A pgp-3 probe detects pgp-3 RNA in both the wild type and nhr-8(ok186) lanes, indicating that expression of pgp-3 does not require intact NHR-8. ama-1 mRNA was detected on the same blot to control for RNA loading and integrity [ama-1 encodes the large subunit of RNA polymerase II, 45]. The size of this RNA is consistent with that predicted for a spliced RNA produced from the remaining 5′ end of the gene. Translation of this RNA would produce a truncated protein containing an intact DBD, which might retain DNA binding capabilities.

Homozygous nhr-8(ok186) animals are viable and grow as well as wild type under standard laboratory conditions, indicating that their ability to digest food and absorb nutrients is not severely impaired. Furthermore, the homozygotes are fertile and produce wild type numbers of progeny, suggesting that yolk synthesis is also not impaired.

### Example 4

### Increased Toxin Sensitivity of nhr-8, pgp-3, and nhr-8;pgp-3 Mutants

Materials and Methods

Toxicity Assays

Assays for nematode killing by selected toxins were performed in polystyrene 12-well tissue culture plates from Costar (Corning, N.Y.). Concentrated stock solutions of toxins were purified by filtration through 0.2 μm nitrocellulose filters (Corning, Corning, N.Y.). Because the toxin concentrations were not measured following filtration, the molarities of the solutions are estimates. Concentrated toxin stock solutions were serially diluted with S media (0.1 M NaCl, 0.05M potassium phosphate, 5 μg/ml cholesterol, 0.05 mM disodium EDTA, 0.025 mM FeSO₄.7H₂O, 0.001 mM MnCl₂.4H₂O, 0.001 mM ZnSO₄.7H₂O, 0.0001 mM CuSO₄.5H₂O, 1 M CaCl₂, and 1 M MgSO₄) to produce appropriate working solutions. To provide a bacterial food source, a saturated culture of E. coli OP50 [43] grown in LB was pelleted and resuspended in each toxin solution. For each 50 ml of toxin solution, 25 ml of the bacterial culture was pelleted for resuspension. Serial dilutions were made in bulk and aliquoted into individual wells (1 ml per well).

To assess killing by the toxins, single hermaphrodites were allowed to lay eggs in individual wells for a 12 hour interval, then transferred to fresh wells. Hatched larvae in each well were counted 12 hours after removal of the hermaphrodite and observed over the course of seven days for development and survival to adulthood [wild type animals complete postembryonic development within 3 days, 29]. Each toxin treatment was assessed on three 12-hour intervals for four hermaphrodites, and the results were averaged.

Results

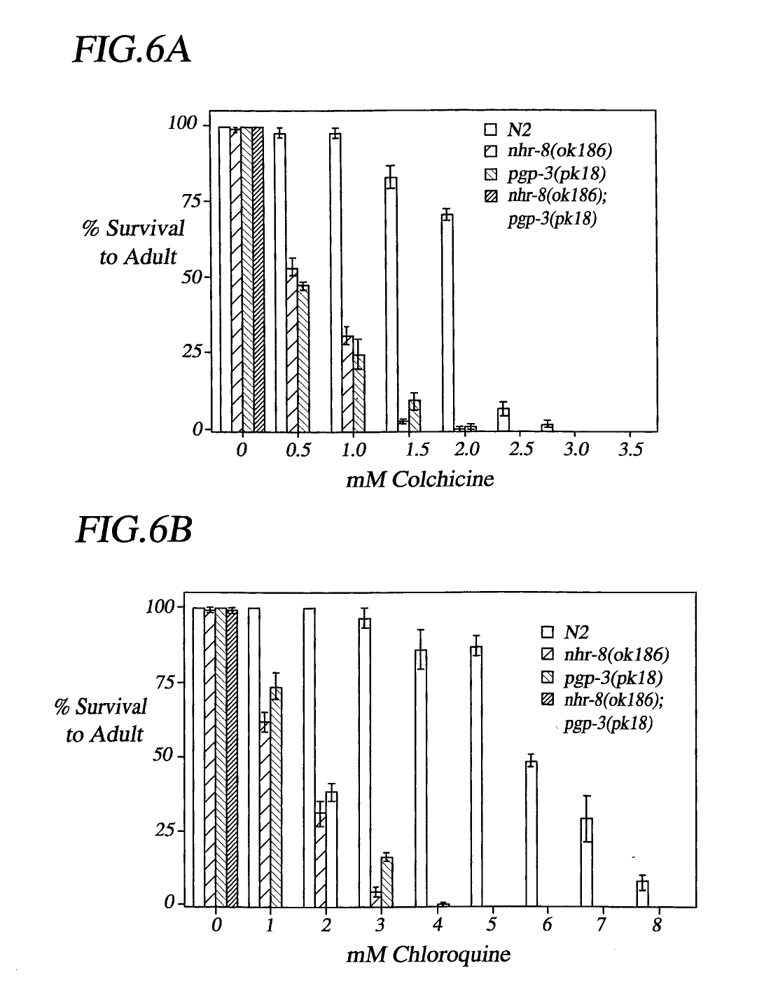

Since NHR-8 is a member of the xenobiotic sensing NR subfamily and is expressed in the gut, the inventors tested nhr-8(ok186) animals for defense against toxins, the third major gut function. Intestinal function has previously been shown to be important for resistance to the plant toxins colchicine and chloroquine [20]. As shown in FIG. 6, nhr-8(ok186) homozygotes are more sensitive than are wild type animals to both compounds. Furthermore, nhr-8(ok186) animals are as sensitive to the toxins as are mutants homozygous for a putative null allele of the ABC transporter gene pgp-3 [FIG. 6; 20].

As discussed in the previous example, the presence of a stable nhr-8 RNA species in nhr-8(ok186) homozygotes suggests that this deletion allele is not a null mutation. Translation of the predicted nhr-8(ok186) RNA would result in a truncated protein with intact A/B (N-terminal) and DNA binding domains. The A/B domain is known to harbor weak, constitutive transactivation activity in some NRs [1] and it is possible that an nhr-8(ok186) protein could bind DNA and activate transcription of target loci, though this protein would lack the LBD and the strong transactivation motif AF-2. However, as shown in the next Example, disruption of nhr-8 function by RNA interference also leads to increased toxin sensitivity, indicating that the phenotype results from a loss of nhr-8 activity and not from neomorphic activity of a truncated nhr-8(ok186) protein. To test the possibility that nhr-8 and pgp-3 are part of a single genetic xenobiotic defense pathway, the inventors assayed nhr-8(ok186); pgp-3(pk18) double mutants for toxin sensitivity. As shown in FIG. 6, the nhr-8(ok186); pgp-3(pk18) double mutant animals were more sensitive to colchicine and chloroquine than were animals with only a single disrupted gene. The graphs in FIG. 6 indicate the percentage of animals surviving to the adult stage vs. increasing concentrations of toxin (as described in Materials and Methods). Each column shows the mean±the standard error of the percentage surviving progeny from 4 hermaphrodites. For some points, the standard errors are too small to be seen. Panel A represents results obtained for colchicine sensitivity, and Panel B represents results for chloroquine sensitivity.

### Example 5

### Increased Toxin Sensitivity Following RNA-mediated Inactivation of nhr-8

Materials and Methods

nhr-8 RNA-Mediated Gene Interference

Single RNA strands were produced with the Ambion MEGAscript in vitro transcription kit, using as template a PCR amplicon (produced from cm14e12 using primers EL-15 and T7) that lacked sequences encoding the NHR-8 DNA binding domain. To produce dsRNA, equal amounts of sense and antisense transcription reactions and 3× injection buffer (20 mM KPO₄ pH 7.5, 3 mM KCitrate pH 7.5, 2% PEG6000) were combined and incubated on a thermal cycler for strand annealing (95° C., 20 sec.; reduced by 2° C. per 10 sec. cycle for 11 cycles). Both gonad arms of young adult hermaphrodites were injected with the dsRNA [25]. Following a 12 hour purge period to remove embryos unlikely to have received a full dose of dsRNA, the injected hermaphrodites were used in the colchicine toxicity assay described above.

Results

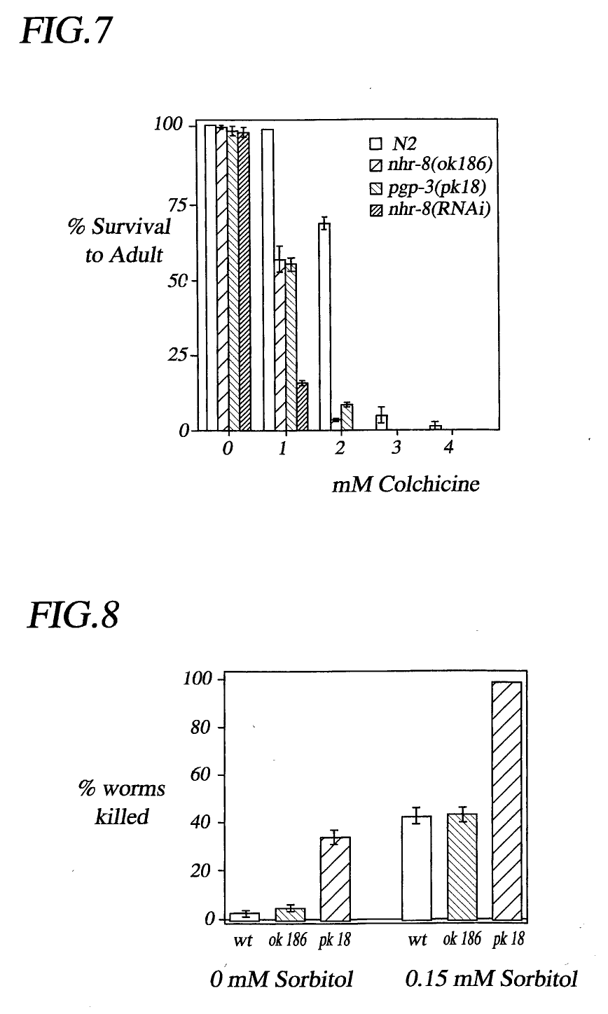

To confirm that the increase in toxin sensitivity is due to the molecular lesion in nhr-8, nhr-8 function was disrupted by RNA mediated interference [25]. To minimize the potential for cross-interference with other NR genes, the double-stranded nhr-8 RNA employed did not include the sequences encoding the conserved DBD. Other than nhr-8, the C. elegans gene most similar to the injected RNA is daf-12 (41% nucleic acid identity), which is required for the development of the dauer larva. Injection of nhr-8 dsRNA does not prevent dauer formation (data not shown) indicating that the effects of the injected RNA are specific to perturbation of nhr-8 function. As shown in FIG. 7, disruption of nhr-8 function by RNA mediated interference phenocopies the colchicine sensitivity of the nhr-8(ok186) mutants, demonstrating that this phenotype is due to loss of nhr-8 function. The graph indicates the percentage of animals surviving to the adult stage vs. increasing concentrations of toxin (as described in Materials and Methods). Each column shows the mean±the standard error of the percentage surviving progeny from 4 hermaphrodites. For some points, the standard errors are too small to be seen.

### Example 6

### Absence of Increased Sensitivity to Fast Killing by P. aeruginosa in nhr-8(ok186) Mutants

P. aerurinosa Fast Killing Assay

P. aeruginosa fast killing assays were performed as described [28]. Wild type, pgp-3(pk18), and nhr-8(ok186) L4 larvae were placed onto lawns of the phenazine-producing P. aeruginosa strain PA14, on both standard nematode plates and those containing 0.15 M sorbitol. Six hours after exposure, lethality was scored as an absence of movement following gentle probing with a sterile platinum wire. Five replicates of ˜15 larvae were performed for each combination of nematode strain and plate composition.

Results

In addition to resistance to ingested xenobiotics, wild type intestinal function is required for the resistance of C. elegans to the pathogenic bacterium P. aeruginosa. This pathogen kills C. elegans by two modes—“fast killing”, which depends upon bacterial production of the aromatic pyocyanin phenazine, and “slow killing”, which results from bacterial proliferation within the nematode gut [26, 27]. Wild type C. elegans is reasonably resistant to fast killing unless exposed to the pathogenic bacteria in a high osmolarity environment. In contrast, nematodes double mutant for the endodermally expressed ABC transporters, pgp-1 and pgp-3, are sensitive to fast-killing even in a low osmolarity environment [28]. The mutants nhr-8(ok186) and pgp-3(pk18) were tested for susceptibility to fast killing by P. aeruginosa. As shown in FIG. 8, loss of PGP-3 alone, but not loss of NHR-8, was found to render nematodes more sensitive than wild type to this bacterial toxicity. The graph shows the percentage of worms killed after 6 hours of exposure to the PA14 strain of P. aeruginosa. Each column is the mean±the standard error of five replicates of 15 L4 larvae. Sorbitol was included to increase the osmolarity of the plates as required for fast killing by P. aeruginosa [26]. While not wishing to be bound by any theory, this observation demonstrates specificity in the toxin sensitivity of nhr-8(ok186) animals.

### Example 7

### Increased Sensitivity of nhr-8(ok186) Mutants to Inducers of Cytochrome P450 Expression

Methods

Assays for nematode sensitivity to toxic compounds known to induce mammalian cytochrome P450 expression (β-naphthaflavone, primaquine, and atrazine, available from Sigma) were performed in polystyrene 24-well tissue culture plates (Costar, Corning, N.Y.). Each culture well was filled with 0.8 ml of NGM agar (Lewis, J. A. and J. T. Fleming, Basic culture methods, in Caenorhabditis elegans: Modern Biological Analysis of an Organism, H. F. Epstein and D. C. Shakes, Editors. 1995, Academic Press: San Diego, Calif. pp. 3-29) containing compound additions as indicated. Compound solutions and dilutions were prepared in DMSO, and the appropriate amounts of compound were added to aliquots of molten NGM agar (equilibrated in a 55° C. water bath) prior to pouring the wells. For each compound, assays were performed using a range of final compound concentrations in the agar (0, 2, 5, 10, 20 and 30 ug/ml β-naphthoflavone; 0, 25, 50, 100, 250, and 77 ug/ml atrazine; 0, 20, 40, 60, 80 and 100 ug/ml primaquine). The final concentration of DMSO in the agar was 1% in every case, and controls of no addition and of 1% DMSO were also included in every assay series. Once the agar was solidified, each well was seeded with a lawn of E. coli OP50-1 prior to the addition of nematodes. All assays were performed in duplicate. Two different assay strategies were used to assess compound effects on nematode growth and viability: L1 assay: Synchrono5us nematode cultures of the wild-type strain N2 and of the mutant nhr-8(ok186) were obtained by isolating L1 larvae as described Lewis, J. A. and J. T. Fleming, Basic culture methods, in Caenorhabditis elegans: Modern Biological Analysis of an Organism, H. F. Epstein and D. C. Shakes, Editors. 1995, Academic Press: San Diego, Calif. pp. 3-29. The synchronized L1 larvae were suspended in M9 and approximately 50 L1 larvae of either N2 or nhr-8(ok186), as indicated, were placed in each assay well. The actual number of worms in each well was determined by direct count. Plates were placed in a plastic container with tight-fitting lid and humidified by including a wet paper towel in the bottom of the plastic container. After incubation at 20° C. for ˜72 hours, larval growth was assessed in each well by counting the number of gravid adults, L4 larvae/non-gravid adults, and larvae younger than the L4 stage. Untreated control cultures routinely contained >90% gravid adults at the time of scoring. For some wells the number of larvae observed at 72 hours was less than the initial number placed on the plate, most likely due to disintegration of arrested animals. This difference was recorded as a “missing” class.

L4 assay: L4 hermaphrodites of the wild-type strain N2 or of the mutant strain nhr-8(ok186) were selected from active growing cultures and placed into the assay wells (4 hermaphrodites/well). Plates were then incubated at 20° C. to allow these L4 larvae to develop to the adult stage and begin laying eggs. The hermaphrodites were removed after ˜18 hours, at which time ˜50 eggs had generally been laid in each well. The actual number of eggs in each well was determined by direct count. After an additional ˜24 hour incubation at 20° C., any embryonic lethality was assessed by counting the number of unhatched eggs remaining in each well; at this time all eggs in untreated control wells were generally hatched. Plates were then incubated at 20° C. for an additional ˜50 hours, and then larval growth was assessed as described for the L1 assay. For some wells the number of larvae observed at 72 hours was less than the initial number placed on the plate, most likely due to disintegration of arrested animals. This difference was recorded as a “missing” class.

Results

To determine if loss of nhr-8 function impaired the nematode's ability to survive exposure to toxic compounds that induce expression of cytochrome P450 genes in other organisms, the investigators assayed the sensitivity of wild-type (N2) and nhr-8(ok186) animals to three such compounds (β-naphthoflavone, primaquine, and atrazine). These compounds have been known for some time to induce expression of CYP450 genes in vertebrates. In vertebrate systems both primaquine and atrazine are likely to be metabolized by CYP450 enzymes (see, e.g., Constantino L, Metabolism of primaquine by liver homogenate fractions. Evidence for monoamine oxidase and cytochrome P450 involvement in the oxidative deamination of primaquine to carboxyprimaquine, Exp Toxicol Pathol 51(4-5):299-303, July 1999; Hanioka N, et al., In vitro metabolism of chlorotriazines: characterization of simazine, atrazine, and propazine metabolism using liver microsomes from rats treated with various cytochrome P450 inducers, Toxicol Appl Pharmacol 1999 May 1, 156(3):195-205.)

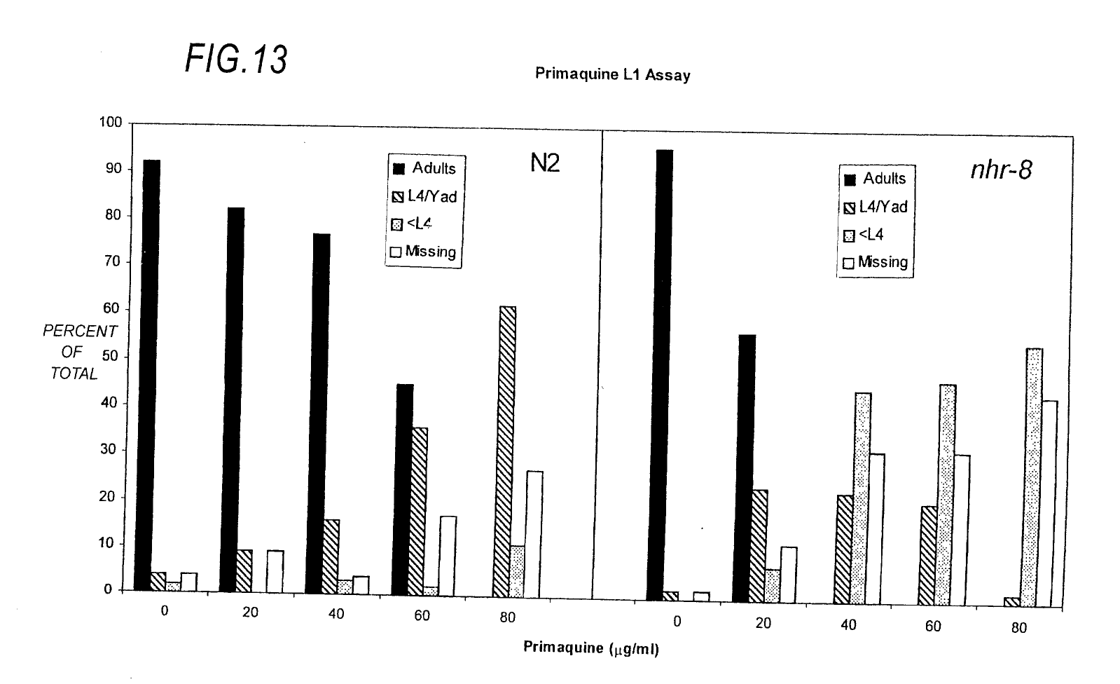

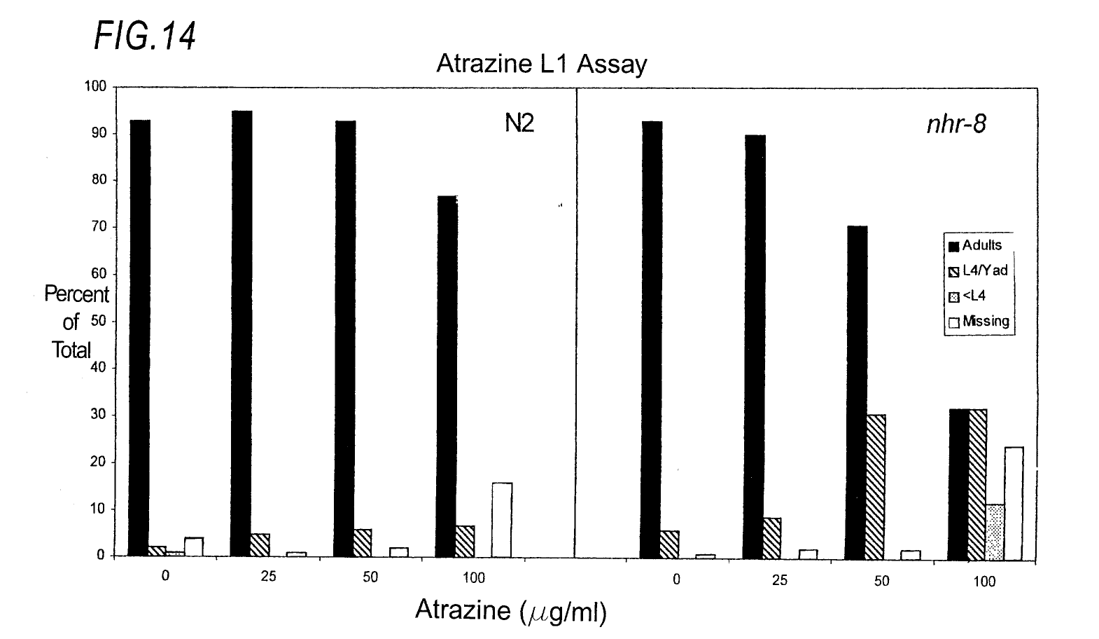

As shown in FIG. 13, nhr-8(ok186) mutant animals tested using the L1 assay exhibited increased sensitivity to primaquine, indicating that loss of nhr-8 function impairs the nematode's defenses against this compounds. Higher compound concentrations at which significant precipitation in the agar was observed are not included in the graph due to uncertainty about the actual concentration of compound available to the exposed nematodes. As shown in FIG. 14, nhr-8(ok186) mutants also exhibited slightly increased sensitivity to atrazine. Higher compound concentrations at which significant precipitation in the agar was observed are not included in the graph due to uncertainty about the actual concentration of compound available to the exposed nematodes. The data in FIGS. 13 and 14 represent experiments in which all wells contained 1% DMSO as compound solvent.

Preliminary experiments did not reveal any detectable differences in the sensitivity of wild-type (N2) and nhr-8(ok186) mutant animals to β-naphthoflavone (data not shown). While not wishing to be bound by any theory, inventors suggest that this may be due to a feature of the metabolism of β-naphthoflavone that is evident in other organisms, i.e., that it is activated to a toxic metabolite by CYP450s. If this is also the case in C. elegans, then a mutation in nhr-8 could have both protective and deleterious effects in terms of sensitivity to β-naphthoflavone.

Results of testing the three compounds using the L4 assay were consistent with those obtained using the L1 assay.

### Example 8

### Induction of Cytochrome P450 Gene Expression in Wild Type C. elegans and in nhr-8(ok186) Mutants

Methods

Synchronous nematode cultures of the wild-type strain N2 and of the mutant nhr-8(ok186) were obtained by isolating L1 larvae as described (Lewis, J. A. and J. T. Fleming, Basic culture methods, in Caenorhabditis elegans: Modern Biological Analysis of an Organism, H. F. Epstein and D. C. Shakes, Editors. 1995, Academic Press: San Diego, Calif. pp. 3-29). For each strain, synchronized L1 larvae were placed on NGM agarose plates (˜2000 larvae/plate) seeded with E. coli OP50-1 (Lewis, J. A. and J. T. Fleming, Basic culture methods, in Caenorhabditis elegans: Modern Biological Analysis of an Organism, H. F. Epstein and D. C. Shakes, Editors. 1995, Academic Press: San Diego, Calif. pp. 3-29). Each plate contained one of the following compounds: no addition; 1% DMSO; 5 ug/ml beta-naphthaflavone plus 1% DMSO; 30 ug/ml atrazine plus 1% DMSO; 20 ug/ml primaquine plus 1% DMSO; 1 mM colchicine; or 1 mM chloroquine. The plates were incubated at 20° C. for ˜72 hours to allow for nematode growth and exposure to compound. Nematodes were then harvested, washed 3× in M9 to remove residual bacteria, and flash frozen in liquid nitrogen. Total RNA was isolated from the frozen nematodes as described (Sluder, A., S. W. Mathews, D. Hough, V. P. Yin, and C. V. Maina, The nuclear receptor superfamily has undergone extensive proliferation and diversification in nematodes. Genome Research, 1999. 9: 103-120).

The levels of cytochrome P450 mRNA in each RNA prep were assayed by semi-quantitative RT-PCR, using primers for specific cytochrome P450 gene sequences presented in Tables 5 and 6, in which nucleotides at degenerate positions are indicated using (N/N). Amplification of act-1 cDNA sequences provided a standard control for the integrity and amount of template in the different cDNA preparations. For 1 ug of each RNA preparation, first strand cDNA was produced using random hexamers to prime cDNA synthesis with MMLV reverse transcriptase (Sluder, A., S. W. Mathews, D. Hough, V. P. Yin, and C. V. Maina, The nuclear receptor superfamily has undergone extensive proliferation and diversification in nematodes. Genome Research, 1999, 9: 103-120). Prior to performing PCR amplification to assess gene expression levels, the number of amplification cycles to be employed with each primer pair was selected to ensure that the amplification reactions remained within the linear reaction range [cycle number was selected as described in Johnstone, I. L. and J. D. Barry, Temporal reiteration of a precise gene expression pattern during nematode development Embo J. 1996, 15: 3633-3639, except that reaction products were analyzed by ethidium bromide-staining rather than Southern blotting]. The amounts of amplification products obtained were quantitated by analyzing ethidium bromide-stained agarose gels with AlphaImager 2200 software (Alpha Innotech Corp.). For each first strand cDNA preparation, the amounts of cytochrome P450 amplification products obtained were normalized to those of the act-1 standard. All PCR reactions were performed in duplicate.

TABLE 5

Forward primers for semi-quantitative RT-PCR

| | |
|---|---|
| Gene | Forward primer sequence (5′-3′) |

| | | |
|---|---|---|
| act-1 | GAGGCCCAATCCAAGAGA | (SEQ ID NO:13) |
| CYP35A1 | TCTTGCGTTGTCATGCTGGT | (SEQ ID NO:14) |
| CYP35A2 | TTCTGTGCTTTTGGGATACC | (SEQ ID NO:15) |
| CYP35A3 | CGCTGCGTGTTTAAGTTGGC | (SEQ ID NO:16) |
| CYP35A4 | TCGGCAATTTTGAGTTGGTT | (SEQ ID NO:17) |
| CYP35A5 | TTCCCTAATTTGACATGGT | (SEQ ID NO:18) |
| CYP35B1 | CTGACCGTCGGAAGTGTTAT | (SEQ ID NO:19) |
| CYP35B2 | GGACTCGGCTTCAGACTTAT | (SEQ ID NO:20) |
| CYP35B3 | TATGACAGTGCCAGTTTGGG | (SEQ ID NO:21) |
| CYP35C | TCCGAAAAGTGATGTTTATG | (SEQ ID NO:22) |
| CYP31A1 | CTCTGGCGACCTTGTTGAAG | (SEQ ID NO:23) |
| CYP31A3 | TACTCCGCCGATCTCGTG | (SEQ ID NO:24) |

TABLE 6

Reverse primers for semi-quantitative RT-PCR

| | |
|---|---|
| Gene | Reverse primer sequence (5′-3′) |

| | | |
|---|---|---|
| act-1 | TGTTGGAAGGTGGAGAGG | (SEQ ID NO:25) |
| CYP35A1 | GCATG(A/G)CGTTGAA(T/C)TCTCC | (SEQ ID NO:26) |
| CYP35A2 | GCATG(A/G)CGTTGAA(T/C)TCTCC | (SEQ ID NO:26) |
| CYP35A3 | GCATG(A/G)CGTTGAA(T/C)TCTCC | (SEQ ID NO:26) |
| CYP35A4 | GCATG(A/G)CGTTGAA(T/C)TCTCC | (SEQ ID NO:26) |
| CYP35A5 | GCATG(A/G)CGTTGAA(T/C)TCTCC | (SEQ ID NO:26) |
| CYP35B1 | AATGAG(A/T)TTCAGCTCTGTCTTT | (SEQ ID NO:27) |
| CYP35B2 | AATGAG(A/T)TTCAGCTCTGTCTTT | (SEQ ID NO:27) |
| CYP35B3 | AATGAG(A/T)TTCAGCTCTGTCTTT | (SEQ ID NO:27) |
| CYP35C | ATGTCGAAGGCTTCGCATCT | (SEQ ID NO:28) |
| CYP31A1 | GAGCCATGATGACCTTCTCT | (SEQ ID NO:29) |
| CYP31A3 | GAGCCATGATGACCTTCTCT | (SEQ ID NO:29) |

Results

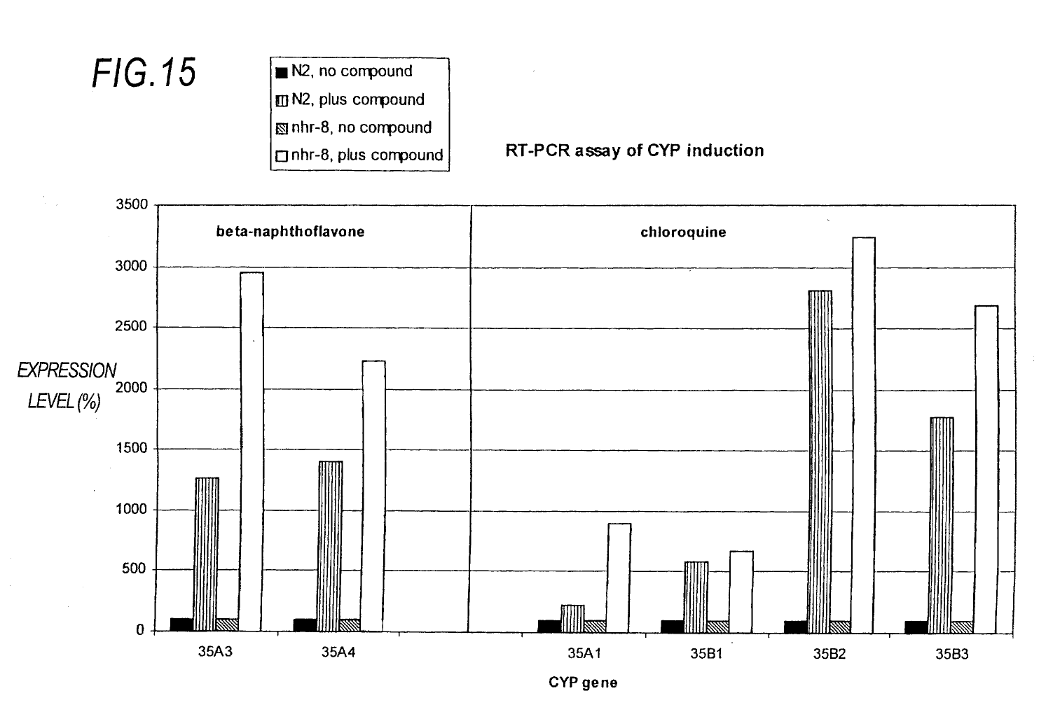

In an initial probe of potential NHR-8 target genes, the inventors assayed the expression of 11 C. elegans CYP genes known to be induced by xenobiotics (R Menzel, T Bogaert, and R Achazi. 2001. A systematic gene expression screen of Caenorhabditis elegans cytochrome P450 genes reveals CYP35 as strongly xenobiotic inducible. Archives of Biochemistry and Biophysics 395: 158-168). Induction of CYP expression was assayed in both N2 and nhr-8(ok186) animals. As shown in FIG. 15, under the xenobiotic exposure conditions utilized (described in Methods), beta-naphthaflavone induced expression of CYP35A3 and CYP35A4. Chloroquine, which was not previously known to induce CYP gene expression in nematodes, induced the expression of CYP35A1, CYP35B1, CYP35B2, and CYP35B3. Induction of these CYP genes was observed in both N2 and nhr-8(ok186) animals. While not wishing to be bound by any theory, these preliminary results suggest that full-length NHR-8 is not required for the upregulation of these six genes upon exposure to beta-naphthaflavone or chloroquine. More comprehensive surveys (for example, by microarray-based expression profiling) of the induction of genes encoding CYPs or other xenobiotic metabolizing proteins in response to a variety of xenobiotics can be utilized to reveal which of the genes encoding xenobiotic metabolizing proteins may be regulated by NHR-8.

### Example 9

### Homology Cloning of an nhr-8 Homolog From H. contortus Genomic DNA

Materials and Methods

Oligonucleotide Primers

Degenerate oligonucleotide primers were designed based on the DNA binding domain (DBD) sequence of the C. elegans NRs NHR-8, NHR-48, and DAF- 12. Degeneracies were accomplished either by incorporation of a mixture of nucleotides or the use of inosine at a specific position in the sequence in order to approximate eight-fold or sixteen-fold degeneracy in the primer sequences. Sequences of the primers used were as follows:

| | | |
|---|---|---|
| NHR8.F1: | 5′-GCIAACGGITAYAAYTTYGG-3′ | (SEQ ID NO:30) |
| NHR8.F3a: | 5′-ACTTGCGAATCNTGYAARGC-3′ | (SEQ ID NO:31) |
| NHR8.R2: | 5′-CATICCIACIGCRAARCAYTT-3′ | (SEQ ID NO:32) |

Amplification Conditions

The candidate nhr-8 sequence was amplified from H. contortus genomic DNA using the degenerate primers described above with a semi-nested polymerase chain reaction (PCR) strategy in a temperature gradient thermocycler (MJ Research PTC-225 Gradient Cycler) consisting of two rounds of amplification. Each 50 μl first round amplification reaction included 2 μl (˜50 ng) of H contortus genomic DNA; 0.4 μl of 25 μM total dNTP; 1 μl of Taq DNA polymerase (5 units/μl, Promega); and 5 μl each of 5 μM NHR8.F1 (forward primer) and 5 μM NHR8.R2 (reverse primer). After an initial 3-minute denaturation at 95° C., reactions were cycled through 35 repetitions of 30 seconds at 95° C.; 1 minute along the following temperature gradient: 50.3° C., 50.8° C., 51.7° C., 52.8° C., 54.3° C., 56.0° C., 57.4° C., and 58.5° C.; and 1 minute at 72° C., incubation at 72° C. for 10 minutes. Each 50 μl second round amplification reaction included 2 μl of first round amplification product; 0.4 μl of 25 μM total dNTP; 1 μl of Taq DNA polymerase (5 units/μl, Promega); and 5 μl each of 5 μM NHR8.F3a (forward primer) and 5 μM NHR8.R2 (reverse primer). The thermocycler parameters were the same as for the first round.

PCR reactions resulted in a ˜200 bp product amplified at an annealing temperature of 51.7° C. This product was gel purified using the Qiaquick Gel Extraction Kit (Qiagen), cloned into the pCR2.1-TOPO vector using the TOPO TA Cloning Kit (Invitrogen), and sequenced at Beth Israel Deaconess Medical Center using universal primer M13 reverse.

Results

Sequencing and subsequent sequence analysis of the 200 bp product revealed that the H. contortus gene is a likely ortholog of nhr-8.

### Example 10

### Creation of Rat GR/NHR-25 LBD NR Protein Chimeras for Use in Yeast Screen for Modulators of NHR-25

This example presents a protocol that was used to adapt two plasmids encoding full-length rat GR to produce modified versions with the rat GR LBD removed and a “Gateway” cloning cassette inserted to allow rapid insertion of nematode NR LBD sequences to produce rat GR/nematode NR LBD chimeras (expression clones). The protocol is referred to as the Expression Clone Strategy. The “Gateway” system is a commercially available system (from Gibco BRL) for rapid cloning of PCR products and subsequent shuttling of inserts into a variety of vectors using in vitro recombination at lambda att sites that are engineered to flank the PCR product. One construct has the CUP1 copper-inducible promoter driving GR/chimera expression; the other has the constitutive GPD promoter. The same protocol is followed to generate NR protein chimeras for other nematode NRs, e.g., nematode xenobiotic sensing NRs such as C. elegans NHR-8, NHR-48, DAF-12, and parasitic nematode homologs, orthologs, or paralogs thereof. It is noted that certain terminology as used in this example (e.g., Entry clone) refers to the instructions that accompany the Gateway system and the protocol generally follows the directions of the manufacturer.

Expression Clone Strategy

Design of Oligonucleotide Primers

Oligonucleotide primers were designed to include sequence specific to the ligand-binding domain (LBD) of the nuclear receptor (NR) of interest as well as the addition of 5′-terminal Gateway Cloning Technology (Life Technologies) 25-bp attB sequences to allow for amplification of a PCR product that would serve as an efficient substrate for the creation of an Entry Clone construct. Primers designed for the amplification of the nhr-25 LBD are used as an example.

The attB sequences used were as follows:

| | | |
|---|---|---|
| attB1: | 5′-GGGGACAAGTTTGTACAAAAAAGCAGGCTNN-3′ | (SEQ ID NO:33) |
| attB2: | 5′-GGGGACCACTTTGTACAAGAAAGCTGGGTN-3′ | (SEQ ID NO:34) |

Ligand binding domain sequences are added to the 3′ ends of both the attB1 and attB2 primers. The particular ligand binding domain sequence varies depending upon the particular NR. The attB1 sequence is added to the forward primer and the attB2 sequence is added to the reverse primer. The attB 1 primer ends with two Ns to indicate that the donation of two additional nucleotides from the remainder of the primer sequence is required in order to maintain the correct reading frame. It is important to note that these two nucleotides cannot be AA, AG, or GA as these combinations would contribute to the creation of a stop codon. The attB2 primer ends with a single N to indicate that this primer requires the donation of one nucleotide from the remainder of the primer in order to maintain the correct reading frame. The four guanine residues at the 5′ end of the attB primers are required by the Gateway Cloning System in order to make it an efficient substrate.

To these attB sequences were added (at the 3 ′end) 18 nhr-25 LBD-specific nucleotides to create the desired primers for amplification of the nhr-25 LBD to be used in the creation of an Entry Clone. The sequences of these desired primers are presented below, with the nhr-25 LBD domain portions underlined.

| | |
|---|---|
| B1 NHR-25: | 5′-GGGGACAAGTTTGTACAAAAAAGCAGGCTtaCATCGTATGCAGAGAAAC-3′ |
| B2 NHR-25: | 5′-GGGGACCACTTTGTACAAGAAAGCTGGGTTTATGATGCCATGTACGG-3′ |

The nucleotides ta indicated in lower case in the B1 NHR-25 primer were included to maintain the correct reading frame.

Amplification Conditions

The candidate nhr-25 LBD sequence was amplified from a 1:1000 dilution of pCB-CP 1 plasmid DNA with polymerase chain reaction (PCR) using the primers described above. The pCB-CP1 construct consists of nhr-25 alpha cDNA full-length coding sequence inserted into a multiple cloning site of the pBS (−) vector. The 50 μl reaction included 2 μl of a 1:1000 dilution of pCB-CP1 plasmid DNA as template; 0.4 μl of 25 mM total dNTP; 1 μl of Taq Plus Precision high fidelity DNA polymerase (5 units/μl, Stratagene); and 5 μl each of 5 μM B1 NHR-25 (forward primer) and 5 μM B2 NHR-25 (reverse primer).

After an initial 3 minute denaturation at 95° C., reactions were cycled through 35 repetitions of 30 seconds at 95° C., 1 minute at 55° C., and 1 minute at 72° C., followed by final incubation at 72° C. for 10 minutes. PCR reactions resulted in a ˜1450 bp product. This product was gel purified using the Qiaquick Gel Extraction Kit (Qiagen) and cloned into the pDONR201 Donor Vector via homologous recombination using the Gateway Cloning Technology (Life Technologies) to create the nhr-25 Entry Clone.

Entry Clone

The attB nhr-25 PCR product was mixed in a BP Clonase reaction with a Donor Vector (pDONR201, Life Technologies) to create the intermediate nhr-25 Entry Clone construct. The BP Clonase reaction takes advantage of homologous recombination between the attB sites engineered into the oligonucleotide primers used to amplify the nhr-25 LBD and the attP sites found in the pDONR201 Donor Vector to create the Entry Clone. Therefore, the Entry clone contains the gene of interest contributed by the attB PCR product (in this case, the nhr-25 LBD) and the Kanamycin resistance gene contributed by the pDONR201 Donor Vector. Recombination between the attB and attP sites results in the creation of attL sites, which contribute to a separate homologous recombination reaction between the newly created Entry Clone and a Destination Vector to create an Expression Clone, construct.

Destination Vector

A four-step process created Destination Vectors. First, a fragment of a yeast expression plasmid containing the rat glucocorticoid receptor (rGR) was generated with PCR. Second, the PCR fragment and the yeast expression plasmid from which it came were digested with appropriate restriction enzymes. Third, the PCR fragment was cloned back into its parent yeast expression plasmid such that the new construct (dubbed the “pre-Destination Vector”) contained a rGR that had been truncated in the hinge domain and allowed for insertion of an appropriate Gateway Reading Frame Cassette (Life Technologies). Finally, the “pre-Destination Vector” then had a Gateway Reading Frame Cassette inserted by blunt-end cloning resulting in the Destination Vector construct. Therefore, the Destination vector contained a rGR truncated in the hinge domain, an Ampicillin resistance gene, a yeast selectable marker, and either a constitutive GPD promoter or a copper-inducible promoter contributed by the parent yeast expression plasmid, plus a Gateway Reading Frame Cassette which contributed attR sites for subsequent homologous recombination reactions that would ultimately result in the Expression Clone containing the gene of interest (i.e. nhr-25 LBD), a Chloramphenicol resistance gene, and a lethal ccdB gene which prevents colony growth when using standard E. coli strains.

Expression Clone

A Destination Vector was mixed with an Entry Clone in a LR Clonase reaction to generate an Expression Clone. The LR Clonase reaction takes advantage of homologous recombination between the attR sites contributed by the Destination Vector and the attL sites contributed by the Entry Clone. Therefore, the Expression Clone contains the rGR truncated in the hinge domain, an Ampicillin resistance gene, a yeast selectable marker, and either a constitutive GPD promoter or a copper-inducible promoter contributed by the Destination Vector and the gene of interest (in this case, the nhr-25 LBD) contributed by the Entry Clone. Colonies containing the by-product plasmid are prevented from growing due to the presence of the lethal ccdB gene as well as a different antibiotic resistance marker.

Both Entry Clones and Expression Clones were generated using the “One-tube” Protocol for Cloning attB-PCR Products Directly Into Destination Vectors (Life Technologies). In the case of nhr-25 LBD, attB nhr-25 PCR product (˜35 ng/μl) was mixed with pDONR201 Donor Vector (150 ng/μl) in a 20 μl BP Clonase reaction and incubated at 25° C. for 4 hours. Following incubation, the reaction was brought up to 30 μl in a LR Clonase reaction. From this 30 μl reaction, 5 μl was immediately removed, 1/10 volume of Proteinase K was added, and the aliquot was incubated at 37° C. for 10 minutes. Library Efficiency DH5a competent cells (Life Technologies) were transformed with 1 μl of this aliquot and plated on LB-Kanamycin (50 μg/μl) plates. Colonies that grew on these plates were the Entry Clones.

The remaining 25 μl of LR reaction was incubated at 25° C. for an additional 2 hours then treated with Proteinase K as above. Library Efficiency DH5α competent cells (Life Technologies) were transformed with 1 μl of this reaction and plated on LB-Ampicillin (50 μg/μl) plates. Colonies that grew on these plates were the Expression Clones.

### Reference List 1

1 Gronemeyer, H. and Laudet, V. (1995) Transcription factors 3: Nuclear receptors. Protein Profile 2, 1173-1308

2 Goffeau, A. et al. (1996) Life with 6000 genes. Science 274, 546-567

3 Escriva, H. et al. (1997) Ligand binding was acquired during evolution of nuclear receptors. Proceedings of the National Academy of Science U.S.A. 94, 6803-6808

4 Arabidopsis Genome Initiative, T. (2000) Analysis of the genome sequence of the flowering plant Arabidopsis thaliana. Nature 408, 796-815

5 Freedman, L. P. (1997) Molecular Biology of Steroid and Nuclear Hormone Receptors, Birkhauser

6 Laudet, V. (1997) Evolution of the nuclear receptor superfamily: early diversification from an ancestral orphan receptor. J Mol Endocrinol 19, 207-226

7 Manglesdorf, D. J. et al. (1995) The nuclear receptor superfamily: The second decade. Cell 83, 835-839

8 Kliewer, S. A. et al. (1999) Orphan nuclear receptors: shifting endocrinology into reverse. Science 284, 757-760

9 Repa, J. J. and Manglesdorf, D. J. (1999) Nuclear receptor regulation of cholesterol and bile acid metabolism. Current Opinion in Biotechnology 10, 557-563

10 Waxman, D. J. (1999) P450 gene induction by structurally diverse xenochemicals: Central role of nuclear receptors CAR, PXR, and PPAR. Archives of Biochemistry and Biophysics 369, 11-23

11 Beato, M. et al. (1995) Steroid hormone receptors: Many actors in search of a plot. Cell 83, 851-857

12 Kastner, P. et al. (1995) Nonsteroid nuclear receptors: What are genetic studies telling us about their role in real life? Cell 83, 859-869

13 Thummel, C. (1995) From embryogenesis to metamorphosis: The regulation and function of Drosophila nuclear receptor superfamily members. Cell 83, 871-877

14 C. elegans Sequencing Consortium, T. (1998) Genome sequence of the nematode C. elegans: A platform for investigating biology. Science 282, 2012-2018

15 Sluder, A. E. et al. (1999) The nuclear receptor superfamily has undergone extensive proliferation and diversification in nematodes. Genome Research 9, 103- 120

16 Miyabayashi, T. et al. (1999) Expression and function of members of a divergent nuclear receptor family in C. elegans. Developmental Biology 215, 314-331

17 Rubin, G. M. et al. (2000) Comparative genomics of the eukaryotes. Science 287, 2204-2215

18 Nuclear Receptors Committee, T. (1999) A unified nomenclature system for the nuclear receptor superfamily. Cell 97, 1-2

19 Blaxter, M. L. et al. (1998) A molecular evolutionary framework for the phylum Nematoda. Nature 392, 71-75

20 Williams, S. A. et al. (2000) The filarial genome project: analysis of the nuclear, mitochondrial and endosymbiont genomes of B. malayi. International Journal for Parasitology 30, 411-419

21 Yates, R. A. et al. (1995) Characterization of genes encoding members of the nuclear hormone receptor superfamily from Onchocerca volvulus. Molecular and Biochemical Parasitology 70, 19-31

22 Moore, J. and Devaney, E. (1999) Cloning and characterization of two nuclear receptors from the filarial nematode Brugia pahangi. Biochemical Journal 344, 245-252

23 Unnasch, T. R. et al. (1999) Characterization of a putative nuclear receptor from Onchocerca volvulus. Molecular and Biochemical Parasitology 104, 259-269

24 McCarter, J. P. et al. (2000) Nematode gene sequences, December 2000 update. Journal of Nematology 32, in press

25 Sengupta, P. et al. (1994) The C. elegans gene odr-7 encodes an olfactory-specific member of the nuclear receptor superfamily. Cell 79, 971-980

26 Zhou, H. M. and Walthall, W. W. (1998) UNC-55, an orphan nuclear hormone receptor, orchestrates synaptic specificity among two classes of motor neurons in Caenorhabditis elegans. Journal of Neuroscience 18, 10438-10444

27 Much, J. W. et al. (2000) The fax-1 nuclear hormone receptor regulates axon pathfinding and neurotransmitter expression. Development 127, 703-712

28 Kobayashi, M. et al. (1999) Identification of a photoreceptor cell-specific nuclear receptor. Proceedings of the National Academy of Science USA. 96, 4814-4819

29 Mlodzik, J. et al. (1990) The Drosophila seven-up gene, a member of the steroid receptor gene superfamily, controls photoreceptor cell fates. Cell 60, 211-224

30 Pereira, F. A. et al. (1995) Chicken ovalbumin upstream promoter transcription factor (COUP-TF): expression during mouse embryogenesis. Journal of steroid Biochemistry and Molecular Biology 53, 503-508

31 Carmi, I. et al. (1998) The nuclear hormone receptor SEX-1 is an X-chromosome signal that determines nematode sex. Nature 396, 168-173

32 Antebi, A. et al. (2000) daf-12 encodes a nuclear receptor that regulates the dauer diapause and developmental age in C. elegans. Genes & Development 14, 1512-1527

33 Antebi, A. et al. (1998) daf-12 regulates developmental age and the dauer alternative in Caenorhabditis elegans. Development 125, 1191-1205

34 Hotez, P. et al. (1993) Hookworm larval infectivity, arrest, and amphiparatensis: The Caenorhabditis elegans Daf-c paradigm. Parasitology Today 9, 23-26

35 Siddiqui, A. A. et al. (2000) A cDNA encoding a nuclear homone receptor of the steroid/thyroid hormone-receptor superfamily from the human parasitic nematode Strongyloides stercoralis. Parasitology Research 86, 24-29

36 Jansen, G. et al. (1997) Reverse genetics by chemical mutagenesis in Caenorhabditis elegans. Nature Genetics 17, 119-121

37 Fire, A. et al. (1998) Potent and specific genetic interference by double-stranded RNA in Caenorhabditis elegans. Nature 391, 806-811

38 Kostrouchova, M. et al. (1998) CHR3: a Caenorhabditis elegans orphan nuclear hormone receptor required for proper epidermal development and molting. Development 125, 1617-1626

39 Asahina, M. et al. (2000) The conserved nuclear receptor Ftz-F1 is required for embryogenesis, moulting and reproduction in Caenorhabditis elegans. Genes to Cells 5, 711-723

40 Gissendanner, C. R. and Sluder, A. E. (2000) nhr-25, the C. elegans orthologue of ftz-f1, is required for epidermal and somatic gonad development. Developmental Biology 221, 259-272

41 Sluder, A. E. et al. (1997) The Caenorhabditis elegans orphan nuclear hormone receptor gene nhr-2 functions in early embryonic development. Dev Biol 184, 303-319

42 Clarke, N. D. and Berg, J. M. (1998) Zinc fingers in Caenorhabditis elegans: Finding families and probing pathways. Science 282, 2018-2022

43 Francoijs, C. J. J. et al. (2000) Sequence annotation of nuclear receptor ligand-binding domains by automated homology modeling. Protein Engineering 13, 391-394

44 Davey, K. G. (1988) Endocrinology of nematodes. In Endocrinology of Selected Invertebrate Types (Laufer, H. and Downer, R.G.H., eds), pp. 63-86, Alan R. Liss, Inc.

45 Bird, A. F. and Bird, J. (1991) The Structure ofNematodes, Academic Press, Inc.

46 Goh, S. L. and Davey, K. G. (1985) Occurrence of noradrenaline in the central nervous system of Phocanema decipiens and its possible role in the control of ecdysis. Canadian Journal of Zoology 63, 475-479

47 Apfeld, J. and Kenyon, C. (1998) Cell nonautonomy of C. elgans DAF-2 function in the regulation of diapause and life span. Cell 95, 199-210

48 Chitwood, D. J. et al. (1983) Novel nuclear methylation of sterols by the nematode Caenorhabditis elegans. Steroids 42, 311-319

49 Salt, T. A. et al. (1986) 24-Methyl-23-dehydrocholesterol: A new sterol intermediate in C-24 demethylation from the nematodes Panagrellus redivivus and Caenorhabditis elegans. Steroids, 451-460

50 Cleator, M. et al. (1987) Identity and tissue localization of free and conjugated ecdysteroids in adults of Dirofilaria immitis and Ascaris suum. Molecular and Biochemical Parasitology 25, 93-105

51 Mercer, J. et al. (1989) Analysis of ecdysteroids in Onchocerca gibsoni, O. volvulus and nodule tissues. Tropical Medicine and Parasitology 40, 434-439

52 Dennis, R. (1976) Insect morphogenetic hormones and developmental mechanisms in the nematode, Nematospiroides dubius. Comparative Biochemistry and Physiology 53, 53-56

53 Fleming, M. (1985) Ascaris suum: role of ecdysteroids in molting. Experimental Parasitology 60, 207-210

54 Barker, G. C. et al. (1990) Ecdysteroids in helminths and annelids. Invertebrate Reproduction and Development 18, 1-11

55 Warbrick, E. V. et al. (1993) The effects of invertebrate hormones and potential hormone inhibitors on the third larval moult of the filarial nematode, Dirofilaria immitis, in vitro. Parasitology 107, 459-463

56 Barker, G. C. et al. (1991) The effect of ecdysteroids on the microfilarial production of Brugia pahangi and the control of meiotic reinitiation in the oocytes of Dirofilaria immitis. Parasitology Research 77, 65-71

57 Barker, G. C. et al. (1989) Effects of potential inhibitors on Brugia pahangi in vitro: macrofilaricidal action and inhibition of microfilarial production. Parasitology 99, 409-416

58 Aguinaldo, A. M. A. et al. (1997) Evidence for a lade of nematodes, arthropods and other moulting animals. Nature 387, 489-493

59 Mushegian, A. R. et al. (1998) Large-scale taxonomic profiling of eukaryotic model organisms: A comparison of orthologous proteins encoded by the human, fly, nematode, and yeast genomes. Genome Research 8, 590-598

60 de Rosa, R. et al. (1999) Hox genes in brachiopods and priapulids and protostome evolution. Nature 399, 772-776

61 Chitwood, D. J. and Feldlaufer, M. F. (1990) Ecdysteroids in axenically propagated Caenorhabditis elegans and culture medium. Journal of Nematology 22, 598-607

62 Riddiford, L. M. (1994) Cellular and molecular actions of juvenile hormone I: General considerations and premetamorphic actions. In Advances in Insect Physiology (Vol. 24) (Vans, P. D., ed.), pp. 213-273, Academic Press

63 Wilson, T. E. et al. (1992) Participation of non-zinc finger residues in DNA binding by two nuclear orphan receptors. Science 256, 107-110

64 Bayaa, M. et al. (2000) The classical progesterone receptor mediates Xenopus oocyte maturation through a nongenomic mechanism. Proceedings of the National Academy of Science U.S.A. 97, 12607-12612

65 Li, H. et al. (2000) Cytochrome c release and apoptosis induced by mitochondrial targeting of nuclear orphan receptor TR3. Science 289, 1159-1164

66 Simoncini, T. et al. (2000) Interaction of oestrogen receptor with the regulatory subunit of phosphatidylinositol-3-OH kinase. Nature 407, 538-541

67 Bird, D. and Riddle, D. L. (1994) A genetic nomenclature for parasitic nematodes. Journal of Nematology 26, 138-143

68 Blaxter, M. L. et al. (1997) A unified nomenclature for filarial genes. Parasitology Today 13, 416-417

### Reference List 2

1. Liu, L X, & P F Weller. 1994. Intestinal nematodes. In: Isselbacher, K J, E Braunwald, J D Wilson, J B Martin, A S Fauci, & D L Kasper, eds. Harrison's Principles of Internal Medicine. New York: McGraw-Hill, 916-920.

2. McMahon, J E, & P E Simonsen. 1996. Filariases. In: Cook, GC, ed. Manson's Tropical Diseases. London: W. B. Sauders Co., Ltd., 1321-1368.

3. David, J R, & L X Liu. 1994. Molecular biology and immunology of parasitic infections. In: Isselbacher, K J, E Braunwald, J D Wilson, J B Martin, A S Fauci, & D L Kasper, eds. Harrison's Principles of Internal Medicine. New York: McGraw-Hill, 865-871.

4. Liu, L X, & P F Weller. 1996. Drug therapy: Antiparasitic drugs. New England Journal of Medicine 334: 1178-1184.

5. Sangster, N C, & J Gill. 1999. Pharmacology of anthelminthic resistance. Parasitology Today 15: 141-146.

6. Drews, J. 1996. Genomic sciences and the medicine of tomorrow. Nature Biotechnology 14: 1516-1518.

7. Epstein, H F, & D C Shakes, 1995. eds. Caenorhabditis elegans: Modern Biological Analysis of an Organism. San Diego, Calif.: Academic Press,

8. Link, E M, G Hardiman, A E Sluder, C D Johnson, & L X Liu. 2000. Therapeutic target discovery using C. elegans. Pharmacogenomics in press:

9. Bürglin, T R, E Lobos, & M L Blaxter. 1998. Caenorhabditis elegans as a model for parasitic nematodes. International Journal for Parasitology 28: 395-411.

10. Hotez, P, J Hawdon, & G A Schad. 1993. Hookworm larval infectivity, arrest, and amphiparatenesis: The Caenorhabditis elegans Daf-c paradigm. Parasitology Today 9: 23-26.

11. Ashton, F T, V M Bhopale, D Holt, G Smith, & G A Schad. 1998. Developmental switching in the parasitic nematode Strongyloides stercoralis is controlled by the ASF and ASI amphidial neurons. Journal of Parasitology 84: 691-695.

12. Sonnhammer, E L, & R Durbin. 1997. Analysis of protein domain families in Caenorhabditis elegans. Genomics 46: 200-216.

13. C. elegans Sequencing Consortium. 1998. Genome sequence of the nematode C. elegans: A platform for investigating biology. Science 282: 2012-2018.

14. Manglesdorf, D J, C Thummel, M Beato, P Herrlich, G Schütz, K Umesono, B Blumberg, P Kastner, M Mark, P Chambon, & R M Evans. 1995,. The nuclear receptor superfamily: The second decade. Cell 83: 835-839.

15. Sluder, A E, S W Mathews, D Hough, V P Yin, & C V Maina. 1999. The nuclear receptor superfamily has undergone extensive proliferation and diversification in nematodes. Genome Research 9: 103-120.

16. Rastinejad, F. 1997. Structure and function of the steroid and nuclear receptor DNA binding domain. In: Freedman, L P, ed. Molecular Biology of Steroid and Nuclear Hormone Receptors. Boston: Birkhauser, 105-131.

17. Simons, S S, Jr. 1997. Structure and function of the steroid and nuclear receptor ligand-binding domains. In: Freedman, L P, ed. Molecular Biology of Steroid and Nuclear Hormone Receptors. Boston: Birkhauser, 35-104.

18. Gronemeyer, H, & V Laudet. 1995. Transcription factors 3: Nuclear receptors. Protein Profile 2: 1173-1308.

19. Mangelsdorf, D J, & R M Evans. 1995. The RXR heterodimers and orphan receptors. Cell 83: 841-850.

20. Forman, B M. 1997. Orphan nuclear receptors and their ligands. In: Freedman, L P, ed. Molecular Biology of Steroid and Nuclear Hormone Receptors. Boston: Birkhauser, 281-305.

21. Gustafsson, J-A. 1999. Seeking ligands for lonely orphan receptors. Science 284: 1285-1286.

22. Chomienne, C, P Fenaux, & L Degos. 1996. Retinoid differentiation therapy in promyelocytic leukemia. FASEB Journal 10: 1025-1030.

23. Lotan, R. 1996. Retinoids in cancer chemoprevention. FASEB Journal 10: 1031 -1039.

24. Forman, B M, P Tontonoz, J Chen, R P Brun, B M Spiegelman, & R M Evans. 1995. 15-Deoxy-Delta-12,14-prostaglandin J2 is a ligand for the adipocyte determination factor PPARgamma. Cell 83: 803-812.

25. Lehmann, J M, L B Moore, T A Smith-Oliver, W O Wilkison, T M Willson, & S A Kliewer. 1995. An antidiabetic thiazolidinedione is a high affinity ligand for peroxisome proliferator-activated receptor γ (PPARγ). Journal of Biological Chemistry 270: 12953-12956.

26. Laudet, V. 1997. Evolution of the nuclear receptor superfamily: early diversification from an ancestral orphan receptor. Journal of Molecular Endocrinology 19: 207-226.

27. Boehm, M F, L Zhang, B A Badea, S K White, D E Mais, E Berger, C M Suto, M E Goldman, & R A Heyman. 1994. Synthesis and structure-activity relationships of novel retinoid X receptor-selective retinoids. Journal of Medicinal Chemistry 37: 2930-2941.

28. Chiellini, G, J Apriletti, H al Yoshihara, J Baxter, R Riberio, & T Scanlan. 1998. A high-affinity subtype-selective agonist ligand for the thyroid hormone receptor. Chemical Biology 5: 299-306.

29. Moore, J, & E Devaney. 1999. Cloning and characterization of two nuclear receptors from the filarial nematode Brugia pahangi. Biochemical Journal 344: 245-252.

30. Yates, R A, R S Tuan, K J Shepley, & T R Unnasch. 1995. Characterization of genes encoding members of the nuclear hormone receptor superfamily from Onchocerca volvulus. Molecular and Biochemical Parasitology 70: 19-31.

31. Unnasch, T R, J Bradley, J Beauchamp, R Tuan, & M W Kennedy. 1999. Characterization of a putative nuclear receptor from Onchocerca volvulus. Molecular and Biochemical Parasitology 104: 259-269.

32. Siddiqui, A A, C S Stanley, P J Skelly, & S L Berk. 2000. A cDNA encoding a nuclear hormone receptor of the steroid/thyroid hormone-receptor superfamily from the human parasitic nematode Strongyloides stercoralis. Parasitology Research 86: 24-29.

33. Lazdins, J, & M Kron. 1999. New molecular targets for filariasis drug discovery. Parasitology Today 15: 305-306.

34. Sengupta, P, H A Colbert, & C I Bargmann. 1994. The C. elegans gene odr-7 encodes an olfactory-specific member of the nuclear receptor superfamily. Cell 79: 971-980.

35. Walthall, W W, & J A Plunkett. 1995. Genetic transformation of the synaptic pattern of a motoneuron class in Caenorhabditis elegans. Journal of Neuroscience 15: 1035-1043.

36. Wightman, B, R Baran, & G Garriga. 1997. Genes that guide growth cones along the C. elegans ventral nerve cord. Development 124: 2571-2580.

37. Carmi, I, J B Kopczynski, & B J Meyer. 1998. The nuclear hormone receptor SEX-1 is an X-chromosome signal that determines nematode sex. Nature 396: 168-173.

38. Yeh, W-Y. 1991. Genes acting late in the signalling pathway for Caenorhabditis elegans dauer larval development [Ph.D. thesis]. University of Missouri, Columbia.

39. Sluder, A E, T Lindblom, & G Ruvkun. 1997. The Caenorhabditis elegans orphan nuclear hormone receptor gene nhr-2 functions in early embryonic development. Dev Biol 184: 303-319.

40. Kostrouchova, M, M Krause, Z Kostrouch, & J E Rall. 1998. CHR3: a Caenorhabditis elegans orphan nuclear hormone receptor required for proper epidermal development and molting. Development 125: 1617-1626.

41. Fire, A, S Xu, M K Montgomery, S A Kostas, S E Driver, & C C Mello. 1998. Potent and specific genetic interference by double-stranded RNA in Caenorhabditis elegans. Nature 391: 806-811.

42. Gissendanner, C R, & A E Sluder. 2000. nhr-25, the C. elegans orthologue of ftz-f1, is required for epidermal and somatic gonad development. Developmental Biology in press:

43. Chitwood, D J, W R Lusby, R Lozano, M J Thompson, & J A Svoboda. 1983. Novel nuclear methylation of sterols by the nematode Caenorhabditis elegans. Steroids 42: 311-319.

44. Salt, T A, R Lozano, W R Lusby, D J Chitwood, & M J Thompson. 1986. 24-Methyl-23-dehydrocholesterol: A new sterol intermediate in C-24 demethylation from the nematodes Panagrellus redivivus and Caenorhabditis elegans. Steroids 451-460.

45. Heyman, R A, D J Mangelsdorf, J A Dyck, R B Stein, G Eichele, R M Evans, & C Thaller. 1992. 9-cis retinoic acid is a high affinity ligand for the retinoid X receptor. Cell 68: 379-406.

46. Levin, A A, L J Sturzenbecker, S Kazmer, T Bosakowski, C. Huselton, G Allenby, J Speck, C Kratzeisen, M Rosenberger, A Lovey, & J F Grippo. 1992. 9-cis retinoic acid stereoisomer binds and activates the nuclear receptor RXRα. Nature 355: 359-361.

47. Lehmann, J M, L Jong, A Fanjul, J F Cameron, X P Lu, P Haefner, M I Dawson, & M Pfahl. 1992. Retinoids selective for retinoid X receptor response pathways. Science 258: 1944-1946.

48. Bourguet, W, M Ruff, P Chambon, H Gronemeyer, & D Moras. 1995. Crystal structure of the ligand-binding domain of the human nuclear receptor RXR-α. Nature 375: 377-382.

49. Renaud, J-P, N Rochel, M Ruff, V Vivat, P Chambon, H Gronemeyer, & D Moras. 1995. Crystal structure of the RAR-γ ligand-binding domain bound to all-trans retinoic acid. Nature 378: 681-689.

50. Wagner, R L, A J W., M E McGrath, B L West, J D Baxter, & R J Fletterick. 1995. A structural role for hormone in the thyroid hormone receptor. Nature 378: 690-697.

51. Brzozowski, A M, A C W Pike, Z Dauter, R E Hubbard, T Bonn, O Engstrom, L Ohman, G L Greene, J-A Gustafsson, & M Carlquist. 1997. Molecular basis of agonism and antagonism in the oestrogen receptor. Nature 389: 753-758.

52. Nolte, R T, G B Wisely, S Westin, J E Cobb, M H Lambert, R Kurokawa, M G Rosenfeld, T M Willson, C K Glass, & M V Milburn. 1998. Ligand binding and co-activator assembly of the peroxisome proliferator-activated receptor-γ. Nature 395: 137-143.

53. Tanenbaum, D M, Y Wang, S P Williams, & P B Sigler. 1998. Crystallographic comparison of the estrogen and progesterone receptors' ligand binding domains. Proc. Natl. Acad. Sci. USA 95: 5998-6003.

54. Uppenberg, J, C Svensson, M Jaki, G Bertilsson, L Jendeberg, & A Berkenstam. 1998. Crystal structure of the ligand binding domain of the human nuclear receptor PPARγ. Journal of Biological Chemistry 273: 31108-31112.

55. Williams, S P, & P B Sigler. 1998. Atomic structure of progesterone complexed with its receptor. Nature 393: 392-396.

56. Wurtz, J-M, W Bourguet, J-P Renaud, V Vivat, P Chambon, D Moras, & H Gronemeyer. 1996. A canonical structure for the ligand-binding domain of nuclear receptors. Nature Structural Biology 3: 87-94.

57. Blaxter, M L, P De Ley, J R Garey, L X Liu, P Scheldeman, A Vierstraete, J R Vanfleteren, L Y Mackey, M Dorris, L M Frisse, J T Vida, & W K Thomas. 1998. A molecular evolutionary framework for the phylum Nematoda. Nature 392: 71-75.

58. Mushegian, A R, J G Garey, J Martin, & L X Liu. 1998. Large-scale taxonomic profiling of eukaryotic model organisms: a comparison of homologous and orthologous proteins encoded by the human, fly, nematode, and yeast genomes. Genome Research 8: 590-598.

59. Frohman, M A, & G R Martin. 1989. Rapid amplification of cDNA ends using nested primers. Technique—A Journal of Methods in Cell and Molecular Biology 1: 165-170.

60. Allegretto, E A, M R McClurg, S B Lazarchik, D L Clemm, S A Kerner, M G Elgort, M F Boehm, S K White, J W Pike, & R A Heyman. 1993. Transactivation properties of retinoic acid and retinoid X receptors in mammalian cells and yeast: Correlation with hormone binding and effects of metabolism. Journal of Biological Chemistry 268: 26625-26633.

61. Hall, B L, Z Smit-McBride, & M L Privalsky. 1993. Reconstitution of retinoid X receptor function and combinatorial regulation of other nuclear hormone receptors in the yeast Saccharomyces cerevisiae. Proceedings of the National Academy of Sciences U.S.A. 90: 6929-6933.

62. McDonnell, D P, J W Pike, D J Drutz, T R Butt, & B W O'Malley. 1989. Reconstitution of the vitamin D-responsive osteocalcin transcription unit in Saccharomyces cerevisiae. Molecular and Cellular Biology 9: 3517-3523.

63. Metzger, D, J H White, & P Chambon. 1988. The human oestrogen receptor functions in yeast. Nature 334: 31-36.

64. Poletti, A, N L Weigel, D P McDonnell, W T Schrader, B W O'Malley, & O M Conneely. 1992. A novel, highly regulated, rapidly inducible system for the expression of chicken progesterone receptor, cPRA, in Saccharomyces cerevisiae. Gene 114: 51-58.

65. Christopherson, K S, M R Mark, V Bajaj, & P J Godowski. 1992. Ecdysteroid-dependent regulation of genes in mammalian cells by a Drosophila ecdysone receptor and chimeric transactivators. Proceedings of the National Academy of Sciences U.S.A. 89: 6314-6318.

66. Eilers, M, D Picard, K R Yamamoto, & J M Bishop. 1989. Chimaeras of Myc oncoprotein and steroid receptors cause hormone-dependent transformation of cells. Nature 340: 66-68.

67. Godowski, P J, D Picard, & K R Yamamoto. 1988. Signal transduction and transcriptional regulation by glucocorticoid receptor-lexA fusion proteins. Science 241: 812-816.

68. Lee, J W, D D Moore, & R A Heyman. 1994. A chimeric thyroid hormone receptor constitutively bound to DNA requires retinoid X receptor for hormone-dependent transcriptional activation in yeast. Molecular Endocrinology 8: 1245-1252.

69. Garabedian, M J, & K R Yamamoto. 1992. Genetic dissection of the signaling domain of a mammalian steroid receptor in yeast. Molecular Biology of the Cell 3: 1245-1257.

70. Schena, M, D Picard, & K R Yamamoto. 1991. Vectors for constitutive and inducible gene expression in yeast. Methods in Enzymology 194: 389-398.

71. Golemis, E, I Serebriiskii, J Gyuris, & R Brent. 1997. Interaction Trap/Two-Hybrid Systems to Identify Interacting Proteins. In: Ausubel, F M, R Brent, R E Kingston, D D Moore, J G Seidman, J A Smith, & K Struhl, ed. Current Protocols in Molecular Biology. New York: John Wiley & Sons, 20.1.1-20.1.35.

72. Brent, R, & M Ptashne. 1984. A bacterial repressor protein or a yeast transcriptional terminator can block upstream activation of a yeast gene. Nature 312: 612-615.

73. Macreadie, I G, O Horaitis, A J Verkuylen, & K W Savin. 1991. Improved shuttle vectors for cloning and high-level Cu(2+)-mediated expression of foreign genes in yeast. Gene 104: 107-111.

74. Yao, T-P, B M Forman, Z Jlang, L Cherbas, J-D Chen, M McKeown, P Cherbas, & R M Evans. 1993. Functional ecdysone receptor is the product of EcR and Ultraspiracle genes. Nature 366: 476-479.

### Reference List 3

1. Gronemeyer H, Laudet V: Transcription factors 3: nuclear receptors. Protein Profile 1995, 2:1173-1308.

2. Wei P, Zhang J, Egan-Hafley M, Liang S, Moore D D: The nuclear receptor CAR mediates specific xenobiotic induction of drug metabolism. Nature 2000, 407:920-923.

3. Kliewer S A, Lehmann J M, Milbum M V, Willson T M: The PPARs and PXRs: nuclear xenobiotic receptors that define novel hormone signaling pathways. Recent Prog Horm Res 1999, 54:345-367.

4. Handschin C, Podvinec M, Meyer U A: CXR, a chicken xenobiotic-sensing orphan nuclear receptor, is related to both mammalian pregnane X receptor (PXR) and constitutive androstane receptor (CAR). Proc Natl Acad Sci USA 2000, 97:10769-10774.

5. Bertilsson G, Heidrich J, Svensson K, Asman M, Jendeberg L, Sydow-Backman M, Ohlsson R, Postlind H, Blomquist P, Berkenstam A: Identification of a human nuclear receptor defines a new signaling pathway for CYP3A induction. Proc Natl AcadSci USA 1998, 95:12208-12213.

6. Blumberg B, Sabbagh W, Jr., Juguilon H, Bolado J, Jr., van Meter C M, Ong E S, Evans R M: SXR, a novel steroid and xenobiotic-sensing nuclear receptor. Genes Dev 1998, 12:3195-3205.

7. Kliewer S A, Moore J T, Wade L, Staudinger J L, Watson M A, Jones S A, McKee D D, Oliver B B, Willson T M, Zetterstrom R H, Perlmann T, Lehmann J M: An orphan nuclear receptor activated by pregnanes defines a novel steroid signaling pathway. Cell 1998, 92:73-82.

8. Lehmann J M, McKee D D, Watson M A, Willson T M, Moore J T, Kliewer S A: The human orphan nuclear receptor PXR is activated by compounds that regulate CYP3A4 gene expression and cause drug interactions. J Clin Invest 1998, 102:1016-1023.

9. Tzameli I, Pissios P, Schuetz E G, Moore D D: The xenobiotic compound 1.4-bis[2-(3,5-dichloropyridyloxy)]benzene is an agonist ligand for the nuclear receptor CAR. Molecular and Cellular Biology 2000, 20:2951-2958.

10. Sluder A E, Mathews S W, Hough D, Yin V P, Maina C V: The nuclear receptor superfamily has undergone extensive proliferation and diversification in nematodes. Genome Research 1999, 9:103-120.

11. Gissendanner C R, Sluder A E: nhr-25, the Caenorhabditis elegans ortholog of ftz-f1, is required for epidermal and somatic gonad development. Dev Biol 2000, 221:259-272.

12. Antebi A, Culotti J G, Hedgecock E M: daf-12 regulates developmental age and the dauer alternative in Caenorhabditis elegans. Development 1998, 125:1191-1205.

13. Antebi A, Yeh W H, Tait D, Hedgecock E M, Riddle D L: daf-12 encodes a nuclear receptor that regulates the dauer diapause and developmental age in C. elegans. Genes Dev 2000, 14:1512-1527.

14. Bargmann C I, Mori I: Chemotaxis and thermotaxis. In: C. elegans II Edited by Riddle D L, Blumenthal T, Meyer B J, Priess J R. pp. 717-738. Plainview, N.Y.: Cold Spring Harbor Laboratory Press; 1997: 717-738.

15. Kotze A C: Cytochrome P450 monooxygenase activity in Haemonchus contortus (Nematoda). Int J Parasitol 1997, 27:33-40.

16. Kotze A C: Peroxide-supported in-vitro cytochrome P450 activities in Haemonchus contortus. Int J Parasitol 1999, 29:389-396.

17. Kotze A C: Oxidase activities in macrocyclic-resistant and -susceptible Haemonchus contortus. J Parasitol 2000, 86:873-876.

18. Gotoh O: Divergent structures of Caenorhabditis elegans cytochrome P450 genes suggest the frequent loss and gain of introns during the evolution of nematodes. Molecular Biology and Evolution 1998, 15:1447-1459.

19. Broeks A, Gerrard B, Allikmets R, Dean M, Plasterk R H: Homologues of the human multidrug resistance genes MRP and MDR contribute to heavy metal resistance in the soil nematode Caenorhabditis elegans. Embo J 1996, 15:6132-6143.

20. Broeks A, Janssen H W, Calafat J, Plasterk R H: A P-glycoprotein protects Caenorhabditis elegans against natural toxins. Embo J 1995, 14:1858-1866.

21. Laudet V: Evolution of the nuclear receptor superfamily: early diversification from an ancestral orphan receptor. J Mol Endocrinol 1997, 19:207-226.

22. Waterston R, Martin C, Craxton M, Huynh C, Coulson A, Hillier L, Durbin R, Green P, Shownkeen R, Halloran N, et al.: A survey of expressed genes in Caenorhabditis elegans. Nat Genet 1992, 1:114-123.

23. Kimble J, Sharrock W J: Tissue-specific synthesis of yolk proteins in Caenorhabditis elegans. Dev Biol 1983, 96:189-196.

24. Lincke C R, Broeks A, The I, Plasterk R H, Borst P: The expression of two P-glycoprotein (pgp) genes in transgenic Caenorhabditis elegans is confined to intestinal cells. Embo J 1993, 12:1615-1620.

25. Fire A, Xu S Q, Montgomery M K, Kostas S A, Driver S E, Mello C C: Potent and specific genetic interference by double-stranded RNA in Caenorhabditis elegans. Nature 1998, 391:806-811.

26. Tan M W, Rahme L G, Stemberg J A, Tompkins R G, Ausubel F M: Pseudomonas aeruginosa killing of Caenorhabditis elegans used to identify P. aeruginosa virulence factors. Proc Natl Acad Sci USA 1999, 96:2408-2413.

27. Tan M W, Mahajan-Miklos S, Ausubel F M: Killing of Caenorhabditis elegans by Pseudomonas aeruginosa used to model mammalian bacterial pathogenesis. Proc Natl Acad Sci USA 1999, 96:715-720.

28. Mahajan-Miklos S, Tan M W, Rahme L G, Ausubel F M: Molecular mechanisms of bacterial virulence elucidated using a Pseudomonas aeruginosa-Caenorhabditis elegans pathogenesis model. Cell 1999, 96:47-56.

29. Wood W B: The Nematode Caenorhabditis elegans. Cold Spring Harbor, N.Y.: Cold Spring Harbor Laboratory; 1988.

30. Avery L, Thomas J H: Feeding and defecation. In: C. elegans II Edited by Riddle D L, Blumenthal T, Meyer B J, Priess J R. pp. 679-716. Plainview, N.Y.: Cold Spring Harbor Laboratory Press; 1997: 679-716.

31. Tan M W, Ausubel F M: Caenorhabditis elegans: a model genetic host to study Pseudomonas aeruginosa pathogenesis. Curr Opin Microbiol 2000, 3:29-34.

32. Escriva H, Delaunay F, Laudet V: Ligand binding and nuclear receptor evolution. Bioessays 2000, 22:717-727.

33. Nelson D R: Metazoan cytochrome P450 evolution. Comp Biochem Physiol C Pharmacol Toxicol Endocrinol 1998, 121:15-22.

34. Thompson J D, Gibson T J, Plewniak F, Jeanmougin F, Higgins D G: The CLUSTAL_X windows interface: flexible strategies for multiple sequence alignment aided by quality analysis tools. Nucleic Acids Res 1997, 25:4876-4882.

35. Wong S M: Orphan nuclear hormone receptors in Caenorhabditis elegans: initial studies of expression andfunction.: University of Georgia; 1997. Masters.

36. Sambrook J, Fritsch E F, Maniatis T: Extraction, purification, and analysis of messenger RNA from eukaryotic cells. In: Molecular cloning: A laboratory manual. pp.7.1-1.83. Plainview, N.Y.: Cold Spring Harbor Laboratory Press; 1989: 7.1-1.83.

37. Dibb N J, Maruyama I N, Krause M, Karn J: Sequence analysis of the complete Caenorhabditis elegans myosin heavy chain gene family. J Mol Biol 1989, 205:603-613.

38. Cassata G, Kagoshima H, Pretot R F, Aspock G, Niklaus G, Burglin T R: Rapid expression screening of Caenorhabditis elegans homeobox open reading frames using a two-step polymerase chain reaction promoter-gfp reporter construction technique. Gene 1998, 212:127-135.

39. Brenner S: The genetics of Caenorhabditis elegans. Genetics 1974, 77:71-94.

40. Mello C, Fire A: DNA Transformation. In: Caenorhabditis elegans: modern biological analysis of an organism Edited by Epstein H F, Shakes D C, vol. 48. pp. xxi, 659. San Diego: Academic Press; 1995: xxi, 659.

41. Liu L X, Spoerke J M, Mulligan E L, Chen J, Reardon B, Westlund B, Sun L, Abel K, Armstrong B, Hardiman G, King J, McCague L, Basson M, Clover R, Johnson C D: High-throughput isolation of Caenorhabditis elegans deletion mutants. Genome Res 1999, 9:859-867.

42. Moerman D G, Baillie D L: Genetic organization in C. elegans: fine-structure analysis of the unc-22 gene. Genetics 1979, 91:95-104.

43. Stiernagle T: Maintenance of C. elegans. In: C. elegans: a practical approach Edited by Hope IA. pp. 51-68. Oxford, England: Oxford University Press; 1999: 51-68.

44. Wurtz J M, Bourguet W, Renaud J P, Vivat V, Chambon P, Moras D, Gronemeyer H: A canonical structure for the ligand-binding domain of nuclear receptors. Nat Struct Biol 1996, 3:206.

45. Bird D M, Riddle D L: Molecular cloning and sequencing of ama-1, the gene encoding the largest subunit of Caenorhabditis elegans RNA polymerase II. Mol Cell Biol 1989, 9:4119-4130.

1. A method of sensitizing a nematode to a xenobiotic comprising:
contacting the nematode with a modulator of a nematode nuclear receptor.

2. A method of sensitizing a nematode to a xenobiotic comprising:
contacting the nematode with a compound that inhibits transcription of gene that encodes a xenobiotic metabolizing enzyme.

3. The method of claim 2, wherein the gene encodes a CYP450 protein.

4. The method of claim 2, wherein the gene encodes a transporter, a hydrolyase, a sulfatase, a glutathione S-transferase, or a UDP-glucuronyltransferase.

5. The method of claim 1, wherein the compound is an antagonist of a nematode nuclear receptor.

6. The method of claim 1, wherein the compound is an agonist of a nematode nuclear receptor.

7. The method of claim 1, wherein the nematode is C. elegans.

8. The method of claim 1, wherein the nematode is a parasitic nematode.

9. The method of claim 8, wherein the nematode nuclear receptor is a homolog of a C. elegans nuclear receptor.

10. The method of claim 8, wherein the nematode nuclear receptor is encoded by a gene that is homologous to a gene encoding a C. elegans nuclear receptor.

11. The method of claim 8, wherein the parasitic nematode is a member of an order selected from the group consisting of the Strongylida, Rhabditida, Ascaridida, Spirurida, Oxyurida, Enoplida, Tylenchida, or Dorylaimida nematode orders.

12. The method of claim 8, wherein the parasitic nematode is a member of a genus selected from the list consisting of the Haemonchus, Oestertagia, Trichostrongylus, Cooperia, Dictyocaulus, Strongylus, Oesophagostomum, Syngamus, Nematodirus, Heligmosomoides, Nippostrongylus, Metastrongylus, Angiostrongylus, Acyclostoma, Necator, Uncinaria, Bunostomum, Strongyloides, Steinernema, Ascaris, Parascaris, Toxocara, Toxascaris, Baylisascaris, Anisakis, Pseudoterranova, Heterakis, Wuchereria, Brugia, Onchocerca, Dirofilaria, Loa, Thelazia, Dracunculus, Gnathostoma, Enterobius, Oxyuris, Syphacia, Trichinella, Trichuris, Capillaria, Globodera, Heterodera, Meloidogyne, Anguina, Ditylenchus, Hirschmanniella, Naccobus, Pratylenchus, Radopholus, Criconema, Tylenchulus, Paratylenchus, Aphelenchus, Bursaphelenchus, Longidorus, Xiphinema, Trichodorus, and Paratrichodorus nematode genera.

13. The method of claim 1, wherein the nematode nuclear receptor is a xenobiotic sensing nuclear receptor.

14. The method of claim 1, wherein the nematode nuclear receptor is a member of the NR1I/J group of nuclear receptors.

15. The method of claim 1, wherein the nematode nuclear receptor is a C. elegans nuclear receptor.

16. The method of claim 15, wherein the C. elegans nuclear receptor is encoded by the nhr-8 gene.

17. The method of claim 15, wherein the C. elegans nuclear receptor is encoded by the nhr-48 gene.

18. The method of claim 15, wherein the C. elegans nuclear receptor is encoded by the daf-12 gene.

19. The method of claim 1, wherein the nematode nuclear receptor is a parasitic nematode nuclear receptor.

20. The method of claim 19, wherein the nematode nuclear receptor is encoded by a parasitic nematode homolog of the C. elegans nhr-8 gene.

21. The method of claim 19, wherein the nematode nuclear receptor is a parasitic nematode homolog of the C. elegans NHR-8 protein.

22. The method of claim 19, wherein the nematode nuclear receptor is encoded by a parasitic nematode homolog of the C. elegans nhr-48 gene.

23. The method of claim 19, wherein the nematode nuclear receptor is a parasitic nematode homolog of the C. elegans NHR-48 protein.

24. The method of claim 19, wherein the nematode nuclear receptor is encoded by a parasitic nematode homolog of the C. elegans daf-12 gene.

25. The method of claim 19, wherein the nematode nuclear receptor is a parasitic nematode homolog of the C. elegans DAF-12 protein.

26. The method of any of claim 21, wherein the homolog is a paralog.

27. The method of any of claim 21, wherein the homolog is an ortholog.

28. A method of sensitizing a nematode to a xenobiotic comprising the step of:
contacting the nematode with a compound that inhibits metabolism, biotransformation, distribution, or elimination of the xenobiotic.

29. The method of claim 28, wherein the compound is a modulator of a nematode xenobiotic sensing nuclear receptor.

30. A method of identifying a compound with anti-nematode activity comprising steps of:
providing a sensitized nematode, wherein the sensitized nematode contains a mutation in a gene encoding a nuclear receptor;
contacting the sensitized nematode with a test compound; and
determining whether an indicator of nematode well-being is altered in the presence of the test compound, wherein an alteration in the indicator of nematode well-being of the sensitized nematode in the presence of the test compound is an indication that the compound possesses anti-nematode activity.

31. The method of claim 30, wherein the indicator of nematode well-being is selected from the group consisting of viability, growth, reproduction, feeding, and movement, and wherein the alteration in the indicator of nematode well-being is a decrease in the indicator.

32. The method of claim 30, wherein the mutation decreases amount or activity of the nuclear receptor.

33. The method of claim 30, wherein the nematode is C. elegans.

34. The method of claim 33, wherein the gene encodes a member of the NR1I/J subfamily of nuclear receptors.

35. The method of claim 33, wherein the gene is the nhr-8 gene.

36. The method of claim 33, wherein the gene is the nhr-48 gene.

37. The method of claim 33, wherein the gene is the daf-12 gene.

38. The method of claim 30, wherein the nematode further contains a mutation in a gene encoding a second nuclear receptor.

39. The method of claim 30, wherein the nematode further contains a mutation in a gene encoding a transporter protein.

40. The method of claim 33, wherein the nematode further contains a mutation in a gene selected from the list consisting of: pgp-1, pgp-2, pgp3, and mrp-1.

41. The method of claim 33, wherein the nematode further contains a mutation in a gene encoding a xenobiotic metabolizing protein.

42. The method of claim 41, wherein the xenobiotic metabolizing protein is a CYP450.

43. The method of claim 30, wherein the mutation results in a decrease in the amount or activity of the nuclear receptor.

44. A method of identifying a compound with anti-nematode activity comprising steps of:
providing a nematode;
contacting the nematode with a modulator of a xenobiotic sensing nuclear receptor, thereby generating a sensitized nematode;
contacting the sensitized nematode with a test compound; and
determining whether an indicator of nematode well-being is altered in the presence of the test compound, wherein an alteration in the indicator of nematode well-being in the presence of the test compound is an indication that the compound possesses anti-nematode activity.

45. The method of claim 44, wherein the indicator of nematode well-being is selected from the group consisting of viability, growth, reproduction, feeding, and movement, and wherein the alteration in the indicator of nematode well-being is a decrease in the indicator.

46. The method of claim 44, wherein the nematode is C. elegans.

47. The method of claim 46, wherein the xenobiotic sensing NR is NHR-8, NHR-48, or DAF-12.

48. The method of claim 44, wherein the modulator is an antagonist, and wherein the amount or activity of the xenobiotic sensing NR in the sensitized nematode is reduced relative to the amount or activity of the xenobiotic sensing NR in the absence of the modulator.

49. A method of identifying a modulator of a nematode nuclear receptor comprising:
providing a first nematode, wherein the first nematode contains a mutation in a gene encoding a nuclear receptor;
providing a second nematode, wherein the second nematode does not have a mutation in the gene encoding a nuclear receptor;
contacting the first and second nematodes with a first compound, wherein contacting the first and second nematodes with the first compound causes a detectable phenotype in the first and second nematodes;
contacting the first and second nematodes with a second compound; and
determining whether the detectable phenotype is altered in the first or second nematodes in the presence of the second compound, wherein a greater alteration in the detectable phenotype in the second nematode relative to any alteration in the detectable phenotype in the first nematode is an indication that the second compound is a modulator of a nematode nuclear receptor.

50. The method of claim 49, wherein the detectable phenotype is a decrease in viability, growth, reproduction, feeding, or movement.

51. The method of claim 49, wherein the mutation decreases amount or activity of the nuclear receptor.

52. A method of identifying a compound that acts synergistically with a modulator of a nematode nuclear receptor comprising steps of:
providing a nematode, wherein the nematode contains a mutation in a gene encoding a nuclear receptor;
contacting the nematode with the compound; and
determining whether an indicator of nematode well-being is altered relative to the indicator in a nematode lacking the mutation in the gene encoding a nematode nuclear receptor, wherein the indicator is assessed in both nematodes in the presence of the compound, and wherein an alteration in the indicator of nematode well-being in the nematode containing the mutation in a nematode nuclear receptor relative to the indicator of nematode well-being in a nematode lacking the mutation in the gene encoding a nematode nuclear receptor is an indication that the compound acts synergistically with a modulator of a nematode nuclear receptor.

53. The method of claim 52, wherein the indicator of nematode well-being is viability, growth, reproduction, movement, or feeding, and wherein the alteration is a decrease in the indicator of nematode well-being.

54. A method of identifying a compound that acts synergistically with an modulator of a nematode nuclear receptor comprising steps of:
providing a nematode, wherein the nematode contains a mutation in a gene encoding a nuclear receptor;
contacting the nematode with the modulator of a nematode nuclear receptor; and
determining whether an indicator of nematode well-being of the nematode in the presence of the compound is altered relative to the indicator of nematode well-being in the presence of the compound, of a nematode that is not contacted with the modulator of the nematode nuclear receptor, wherein an alteration in the indicator of nematode well-being of the nematode contacted with both the modulator of the nematode nuclear receptor and the compound relative to indicator of nematode well-being of a nematode contacted with the compound but not with the modulator of a nematode nuclear receptor is an indication that the compound acts synergistically with an antagonist of a nematode nuclear receptor.

55. The method of claim 52, wherein the indicator of nematode well-being is viability, growth, reproduction, movement, or feeding, and wherein the alteration is a decrease in the indicator of nematode well-being.

56. The method of claims 52 or 54, wherein the modulator of a nematode nuclear receptor is an antagonist of the nematode nuclear receptor.

57. The method of claims 52 or 54 wherein the nematode nuclear receptor is a nematode xenobiotic sensing nuclear receptor.

58. The method of claims 52 or 54, wherein the nematode is C. elegans.

59. The method of claim 58, wherein the nematode nuclear receptor is NHR-8, NHR-48, or DAF-12.

60. A method of generating a nematode comprising steps of:
(a) selecting a parasitic nematode nuclear receptor;
(b) identifying a C. elegans homolog of the receptor selected in step (a);
(c) identifying a nucleic acid comprising a regulatory region of a C. elegans gene encoding the C. elegans homolog identified in step (b); and
(d) generating a transgenic nematode, wherein the cells of the transgenic nematode comprise a nucleic acid sequence encoding a readily detectable marker operably linked to a nucleic acid sequence including the regulatory region identified in step (c).

61. The method of claim 60, wherein the nematode nuclear receptor is a member of the NR1I/J subfamily of nuclear receptors.

62. A method of treating or reducing the likelihood of nematode infection in an individual comprising the steps of:
identifying an individual at risk of or suffering from a nematode infection; and
administering to the individual a first compound that sensitizes the nematode to a second compound;
administering the second compound to the individual.

63. A method of preventing or reducing nematode infestation of a plant comprising the steps of:
identifying a plant at risk of infestation by a nematode;
administering to the plant a first compound that sensitizes a nematode to a second compound; and
administering the second compound to the plant.

64. A method of preventing or reducing nematode infestation of a plant comprising the steps of:
treating soil in which a plant is to be grown with a first compound that sensitizes a nematode to a second compound;
treating soil in which the plant is to be grown with the second compound.

65. A method of preventing or reducing nematode infestation of a plant comprising the steps of:
treating a seed from which a plant is to be grown with a first compound that sensitizes a nematode to a second compound; and
treating a seed from which the plant is to be grown with the second compound.

66. The method of any of claims 62, 63, 64, 65, wherein the first and second compounds are administered together.

67. The method of any of claims 62, 63, 64, 65, wherein one of the compound comprises a modulator of a nuclear receptor.

68. The method of claim 67, wherein the nuclear receptor is a parasitic nematode homolog of a nuclear receptor encoded by the C. elegans nhr-8 gene.

69. The method of claim 67, wherein the nuclear receptor is a parasitic nematode homolog of a nuclear receptor encoded by the C. elegans nhr-48 gene.

70. The method of claim 67, wherein the nuclear receptor is a parasitic nematode homolog of a nuclear receptor encoded by the C. elegans daf-12 gene.

71. The method of claim 67, wherein the nuclear receptor is a parasitic nematode nuclear receptor that is a member of the NR1I/J subfamily of nuclear receptors.

72. A method of treating or reducing the likelihood of nematode infection in an individual comprising the steps of:
identifying an individual at risk of or suffering from a nematode infection; and
administering to the individual a modulator of a nematode nuclear receptor.

73. A method of preventing or reducing nematode infestation of a plant comprising the steps of:
identifying a plant at risk of infestation by a nematode; and
administering to the plant a modulator of a nematode nuclear receptor.

74. A method of preventing or reducing nematode infestation of a plant comprising the step of:
treating soil in which a plant is to be grown with an agonist or antagonist of a nematode nuclear receptor or treating a seed from which a plant is to be grown with a modulator of a nematode nuclear receptor.

75. The method of any of claims 72, 73, or 74, wherein the modulator is an antagonist or a nematode nuclear receptor.

76. The method of any of claims 72, 73, or 74, wherein the modulator is an agonist of a nematode nuclear receptor.

77. A method of identifying a modulator of a nematode nuclear receptor comprising steps of:
providing a cell, wherein the cell expresses a polypeptide comprising a ligand binding domain of a nematode nuclear receptor or a portion thereof and a DNA binding domain or portion thereof, and wherein the cell contains a reporter comprising a reporter gene and a nucleic acid that comprises a binding site for the polypeptide, wherein the nucleic acid is operably linked to the reporter gene;
contacting the cell with a test compound;
determining whether the amount or activity of the reporter is increased or decreased in the presence of the test compound, wherein an increase or decrease in the amount or activity of the reporter is an indication that the test compound is a modulator of a nematode nuclear receptor.

78. The method of claim 77, wherein the nematode nuclear receptor is found naturally in a parasitic nematode that is a member of an order selected from the group consisting of the Strongylida, Rhabditida, Ascaridida, Spirurida, Oxyurida, Enoplida, Tylenchida, or Dorylaimida nematode orders.

79. The method of claim 77, wherein the nematode nuclear receptor is found naturally in a parasitic nematode that is a found naturally in a nematode that is a member of a genus selected from the list consisting of the Haemonchus, Oestertagia, Trichostrongylus, Cooperia, Dictyocaulus, Strongylus, Oesophagostomum, Syngamus, Nematodirus, Heligmosomoides, Nippostrongylus, Metastrongylus, Angiostrongylus, Acyclostoma, Necator, Uncinaria, Bunostomum, Strongyloides, Steinernema, Ascaris, Parascaris, Toxocara, Toxascaris, Baylisascaris, Anisakis, Pseudoterranova, Heterakis, Wuchereria, Brugia, Onchocerca, Dirofilaria, Loa, Thelazia, Dracunculus, Gnathostoma, Enterobius, Oxyuris, Syphacia, Trichinella, Trichuris, Capillaria, Globodera, Heterodera, Meloidogyne, Anguina, Ditylenchus, Hirschmanniella, Naccobus, Pratylenchus, Radopholus, Criconema, Tylenchulus, Paratylenchus, Aphelenchus, Bursaphelenchus, Longidorus, Xiphinema, Trichodorus, and Paratrichodorus nematode genera.

80. The method of claim 77, wherein the reporter gene encodes a detectable marker.

81. The method of claim 77, wherein the reporter gene comprises the E. coli lacZ gene or a functional portion thereof.

82. The method of claim 80, wherein the detectable marker is selected from the list consisting of: a fluorescent polypeptide, a chemiluminescent polypeptide, an epitope tag, and an enzyme.

83. The method of claim 80, wherein the detectable marker is selected from the list consisting of: green fluorescent protein, luciferase, chloramphenicol acetyl transferase, xanthine-guanine phosphoribosyl transferase, beta-galactosidase, a Myc tag, and an HA tag.

84. The method of claim 80, wherein the detectable marker comprises a variant of a marker selected from the list consisting of: green fluorescent protein, luciferase, chloramphenicol acetyl transferase, xanthine-guanine phosphoribosyl transferase, beta-galactosidase, a Myc tag, and an HA tag, wherein the variant is detectable using the same detection means by which the marker of which it is a variant is detectable

85. The method of claim 77, wherein the DNA binding domain binds to the binding site.

86. The method of claim 77, wherein the polypeptide further comprises a transcriptional activation domain.

87. The method of claim 77, wherein the nucleic acid further comprises a promoter operably linked to the reporter gene.

88. The method of claim 87, wherein the promotor is regulatable.

89. The method of claim 88, wherein the promoter is a copper-inducible promoter.

90. The method of claim 87, wherein the promoter is operably linked to the binding site.

91. The method of claim 77, wherein the cell comprises a nucleic acid that encodes the polypeptide.

92. The method of claim 77, wherein the nematode nuclear receptor is a xenobiotic sensing nuclear receptor.

93. The method of claim 77, wherein the nematode nuclear receptor is a C. elegans nuclear receptor.

94. The method of claim 77, wherein the nematode nuclear receptor is a parasitic nematode nuclear receptor.

95. The method of claim 94, wherein the nematode nuclear receptor is a parasitic nematode homolog of a C. elegans nuclear receptor.

96. The method of claim 77, wherein the nematode nuclear receptor is a member of the NR1I/J subfamily of nuclear receptors.

97. The method of claim 77, wherein the nematode nuclear receptor is encoded by the C. elegans nhr-8 gene.

98. The method of claim 77, wherein the nematode nuclear receptor is a parasitic nematode homolog of a nuclear receptor encoded by the C. elegans nhr-8 gene.

99. The method of claim 77, wherein the nematode nuclear receptor is encoded by the C. elegans nhr-48 gene.

100. The method of claim 77, wherein the nematode nuclear receptor is a parasitic nematode homolog of a nuclear receptor encoded by the C. elegans nhr-48 gene.

101. The method of claim 77, wherein the nematode nuclear receptor is encoded by the C. elegans daf-12 gene.

102. The method of claim 77, wherein the nematode nuclear receptor is a parasitic nematode homolog of a nuclear receptor encoded by the C. elegans daf-12 gene.

103. The method of any of claims 95, 98, or 102, wherein the homolog is a paralog.

104. The method of any of claims 95, 98, or 102, wherein the homolog is an ortholog.

105. The method of claim 77, wherein the cell is a yeast cell.

106. The method of claim 77, wherein the cell is a mammalian cell or an insect cell.

107. A method of identifying a genetic target for a compound comprising the steps of:
contacting a population of nematodes with the compound, wherein the nematodes have a mutation in a gene encoding a xenobiotic sensing nuclear receptor, and wherein contacting a nematode with the compound causes a detectable phenotype in the nematode;
mutagenizing the population of nematodes;
identifying a mutant that is resistant to the compound, wherein the resistance is manifested by the absence of the detectable phenotype in the mutant.

108. The method of claim 107, further comprising the step of:
cloning the gene that is mutated in the mutant identified in the identifying step, thereby identifying a genetic target for the compound.

109. The method of claim 107, wherein the detectable phenotype is an indicator of nematode well-being.

110. The method of claim 109, wherein the indicator of nematode well-being is viability, growth, reproduction, feeding, or movement.

111. The method of claim 107, wherein the nematode is C. elegans.

112. A method of identifying a combination of compounds having anti-nematode activity comprising steps of:
contacting the nematode with a first compound that modulates activity or amount of a xenobiotic sensing nuclear receptor;
contacting the nematode with a second compound; and
determining whether an indicator of nematode well-being of the nematode is altered in the presence of both the first compound and the second compound to a greater extent than in the presence of only the first compound or the second compound.

113. The method of claim 112, wherein the first compound comprises an antagonist of the nematode xenobiotic sensing nuclear receptor.

114. The method of claim 112, wherein a greater alteration in the indicator of nematode well-being in the presence of the first compound and the second compound than in the presence of either the first compound or the second compound, is an indication that the combination of first and second compounds possesses anti-nematode activity.

115. A method of identifying a combination of compounds having synergistic anti-nematode activity comprising:
the method of claim 112, wherein one or both of the first and second compounds causes an alteration in an indicator of nematode well-being, further comprising the step of:
determining an expected alteration in the indicator of nematode well-being of the nematode due to the combination of first and second compounds by adding an alteration of the indicator of nematode well-being of the nematode caused by the first compound and an alteration of an indicator of nematode well-being of the nematode caused by the second compound, wherein a greater alteration in the indicator of nematode well-being of the nematode in the presence of a combination of both first and second compounds than the expected alteration is an indication that the combination of compounds has synergistic anti-nematode activity.

116. The method of any of claims 112, 114, or 115, wherein the indicator of nematode well-being is viability, growth, reproduction, movement, or feeding, and wherein the alteration in an indicator of nematode well-being is a decrease in the indicator of nematode well-being.

117. A method of treating or reducing the likelihood of nematode infection in an individual comprising the steps of:
identifying an individual at risk of or suffering from a nematode infection; and
administering to the individual a first compound that sensitizes the nematode to a second compound, wherein the first compound is identified according to the method of claim 77;
administering the second compound to the individual.

118. A method of preventing or reducing nematode infestation of a plant comprising the steps of:
identifying a plant at risk of infestation by a nematode;
administering to the plant a first compound that sensitizes a nematode to a second compound, wherein the first compound is identified according to the method of claim 77; and
administering the second compound to the plant.

119. A method of preventing or reducing nematode infestation of a plant comprising the steps of:
treating soil in which a plant is to be grown with a first compound that sensitizes a nematode to a second compound, wherein the first compound is identified according to the method of claim 77;
treating soil in which the plant is to be grown with the second compound.

120. A method of preventing or reducing nematode infestation of a plant comprising the steps of:
treating a seed from which a plant is to be grown with a first compound that sensitizes a nematode to a second compound, wherein the first compound is identified according to the method of claim 77; and
treating a seed from which the plant is to be grown with the second compound.

121. The method of claim 118, wherein the first and second compounds are administered together.

122. The method of claim 118, wherein the first compound comprises a modulator of a nuclear receptor.

123. The method of claim 122, wherein the nuclear receptor is a parasitic nematode homolog of a nuclear receptor encoded by the C. elegans nhr-8 gene.

124. The method of claim 122, wherein the nuclear receptor is a parasitic nematode homolog of a nuclear receptor encoded by the C. elegans nhr-48 gene.

125. The method of claim 122, wherein the nuclear receptor is a parasitic nematode homolog of a nuclear receptor encoded by the C. elegans daf-12 gene.

126. The method of claim 122, wherein the nuclear receptor is a parasitic nematode nuclear receptor that is a member of the NR1I/J subfamily of nuclear receptors.

127. A method of treating or reducing the likelihood of nematode infection in an individual comprising the steps of:
identifying an individual at risk of or suffering from a nematode infection; and
administering to the individual an antagonist of a nematode nuclear receptor, wherein the antagonist of a nematode nuclear receptor is identified according to the method of claim 77.

128. A method of treating or reducing the likelihood of nematode infection in an individual comprising the steps of:
identifying an individual at risk of or suffering from a nematode infection; and
administering to the individual an agonist of a nematode nuclear receptor, wherein the agonist of a nematode nuclear receptor is identified according to the method of claim 77.

129. A method of preventing or reducing nematode infestation of a plant comprising the steps of:
identifying a plant at risk of infestation by a nematode; and
administering to the plant an antagonist of a nematode nuclear receptor, wherein the antagonist of a nematode nuclear receptor is identified according to the method of claim 77.

130. A method of preventing or reducing nematode infestation of a plant comprising the steps of:
identifying a plant at risk of infestation by a nematode; and
administering to the plant an agonist of a nematode nuclear receptor, wherein the agonist of a nematode nuclear receptor is identified according to the method of claim 77.

131. A method of preventing or reducing nematode infestation of a plant comprising the step of:
treating soil in which a plant is to be grown with an antagonist of a nematode nuclear receptor, wherein the antagonist of a nematode nuclear receptor is identified according to the method of claim 77.

132. A method of preventing or reducing nematode infestation of a plant comprising the step of:
treating soil in which a plant is to be grown with an agonist of a nematode nuclear receptor, wherein the agonist of a nematode nuclear receptor is identified according to the method of claim 77.

133. A method of preventing or reducing nematode infestation of a plant comprising the step of:
treating a seed from which a plant is to be grown with an antagonist of a nematode nuclear receptor, wherein the antagonist of a nematode nuclear receptor is identified according to the method of claim 77.

134. A method of preventing or reducing nematode infestation of a plant comprising the step of:
treating a seed from which a plant is to be grown with an agonist of a nematode nuclear receptor, wherein the agonist of a nematode nuclear receptor is identified according to the method of claim 77.

135. A method of identifying a compound comprising steps of:
providing a sensitized nematode;
contacting the sensitized nematode with a test compound; and
determining whether a nematode phenotype is altered in the presence of the test compound, wherein an alteration in the nematode phenotype of the sensitized nematode in the presence of the test compound is an indication that the compound possesses activity.

136. The method of claim 135, wherein the sensitized nematode contains a mutation in a gene encoding a nuclear receptor.

137. The method of claim 135, wherein the sensitized nematode is a member of a nematode screening strain useful in a nematode model screening system, and wherein the phenotype is a phenotype of interest in the context of such a screening system.

138. The method of claim 136, wherein the mutation decreases amount or activity of the nuclear receptor.

139. The method of claim 135, wherein the nematode is C. elegans.

140. The method of claim 136, wherein the gene encodes a member of the NR1I/J subfamily of nuclear receptors.

141. The method of claim 33, wherein the gene is the nhr-8 gene.

142. The method of claim 139, wherein the gene is the nhr-48 gene.

143. The method of claim 139, wherein the gene is the daf-12 gene.

144. The method of claim 136, wherein the nematode further contains a mutation in a gene encoding a second nuclear receptor.

145. A method of enhancing sensitivity of a nematode screening strain comprising:
inactivating or inhibiting the activity of a nematode nuclear receptor.

146. The method of claim 145, wherein the step of inactivating or inhibiting comprises genetically modifying the nematode screening strain.

147. The method of claim 145, wherein the step of inactivating or inhibiting comprises crossing the nematode screening strain with a nematode strain bearing a mutation or deletion in a gene encoding a nematode nuclear receptor.

148. A sensitized nematode screening strain generated according to the method of claim 145.
